(19) United States
(12) Patent Application Publication
(10) Pub. No.: US 2023/0227539 A1
(43) Pub. Date: Jul. 20, 2023

(54) METHODS AND COMPOSITIONS RELATED TO NEUTRALIZING ANTIBODIES AGAINST HUMAN CORONAVIRUS

(71) Applicant: HIFIBIO (HK) LIMITED, Central (CN)

(72) Inventors: Liang Schweizer, Cambridge, MA (US); Bingqing Shen, Shanghai (CN); Mingjie Chen, Shanghai (CN); Yun-Yueh Lu, Shanghai (CN); He Zhou, Shanghai (CN); Francisco Adrian, Cambridge, MA (US); Qian Zhang, Cambridge, MA (US)

(21) Appl. No.: 18/010,369

(22) PCT Filed: Jun. 16, 2021
(86) PCT No.: PCT/US2021/037615

§ 371 Date: Dec. 14, 2022

(30) Foreign Application Priority Data
Jun. 16, 2020 (WO) PCT/CN2020/096360

(51) Int. Cl. C07K 16/10; A61P 31/14

(52) U.S. Cl. CPC C07K 16/10; A61P 31/14; C07K 2317/565; C07K 2317/55; C07K 2317/622; C07K 2317/92; A61K 2039/505

(57) ABSTRACT

The invention described herein provides neutralizing antibodies against SARS-CoV-2 antigens (such as the S1 subunit of the S antigen) for use in treating human patients having COVID-19.

### REFERENCE TO RELATED APPLICATION

This International Patent Application claims priority to International Patent Application No. PCT/CN2020/096360, filed on Jun. 16, 2020, the entire contents of which are incorporated herein by reference.

### BACKGROUND OF THE INVENTION

The Coronavirus Disease 2019 (COVID-19), caused by the novel SARS-CoV-2 coronavirus, has quickly grown into a global pandemic and a major public health crisis. Since the virus has only recently emerged from bats and crossed over into humans, little is known about the immune response generated against it by infected patients.

Thus, a better understanding of the pathological mechanisms caused by the virus, and development of new therapeutic agents against it are urgently needed.

### SUMMARY OF THE INVENTION

One aspect of the invention provides an isolated or recombinantly produced monoclonal antibody, or an antigen-binding fragment thereof, wherein said monoclonal antibody or antigen-binding fragment thereof is specific for the Spike protein or S protein of SARS-CoV-2, and wherein said monoclonal antibody specifically binds to and/or has a residue within 4 Å of residues T415, G416, K417, D420, Y421, Y453, L455, F456, R457, K458, N460, Y473, Q474, A475, G476, S477, F486, N487, Y489, Q493, S494, Y495, G496, Q498, T500, N501, G502, and Y505 of the S protein, optionally, said monoclonal antibody does not bind to and/or has no residue within 4 Å of residues G446 and Y449 of the S protein.

Another aspect of the invention provides an isolated or recombinantly produced monoclonal antibody, or an antigen-binding fragment thereof, wherein said monoclonal antibody or antigen-binding fragment thereof is specific for an antigen (e.g., the Spike protein or S protein responsible for ACE2 binding) of SARS-CoV-2, and wherein said monoclonal antibody comprises: (1a) a heavy chain variable region (HCVR), comprising a HCVR CDR1 sequence of SEQ ID NO: 1, a HCVR CDR2 sequence of SEQ ID NO: 2, and a HCVR CDR3 sequence of SEQ ID NO: 3; and, (1b) a light chain variable region (LCVR), comprising a LCVR CDR1 sequence of SEQ ID NO: 4, a LCVR CDR2 sequence of SEQ ID NO: 5, and a LCVR CDR3 sequence of SEQ ID NO: 6; or (2a) a heavy chain variable region (HCVR), comprising a HCVR CDR1 sequence of SEQ ID NO: 11, a HCVR CDR2 sequence of SEQ ID NO: 12, and a HCVR CDR3 sequence of SEQ ID NO: 13; and, (2b) a light chain variable region (LCVR), comprising a LCVR CDR1 sequence of SEQ ID NO: 14, a LCVR CDR2 sequence of SEQ ID NO: 15, and a LCVR CDR3 sequence of SEQ ID NO: 16; or (3a) a heavy chain variable region (HCVR), comprising a HCVR CDR1 sequence of SEQ ID NO: 21, a HCVR CDR2 sequence of SEQ ID NO: 22, and a HCVR CDR3 sequence of SEQ ID NO: 23; and, (3b) a light chain variable region (LCVR), comprising a LCVR CDR1 sequence of SEQ ID NO: 24, a LCVR CDR2 sequence of SEQ ID NO: 25, and a LCVR CDR3 sequence of SEQ ID NO: 26; or (4a) a heavy chain variable region (HCVR), comprising a HCVR CDR1 sequence of SEQ ID NO: 31, a HCVR CDR2 sequence of SEQ ID NO: 32, and a HCVR CDR3 sequence of SEQ ID NO: 33; and, (4b) a light chain variable region (LCVR), comprising a LCVR CDR1 sequence of SEQ ID NO: 34 or SEQ ID NO: 115, a LCVR CDR2 sequence of SEQ ID NO: 35, and a LCVR CDR3 sequence of SEQ ID NO: 36; (5a) a heavy chain variable region (HCVR), comprising a HCVR CDR1 sequence of SEQ ID NO: 51, a HCVR CDR2 sequence of SEQ ID NO: 52, and a HCVR CDR3 sequence of SEQ ID NO: 53; and, (5b) a light chain variable region (LCVR), comprising a LCVR CDR1 sequence of SEQ ID NO: 54, a LCVR CDR2 sequence of SEQ ID NO: 55, and a LCVR CDR3 sequence of SEQ ID NO: 56; or (6a) a heavy chain variable region (HCVR), comprising a HCVR CDR1 sequence of SEQ ID NO: 61, a HCVR CDR2 sequence of SEQ ID NO: 62, and a HCVR CDR3 sequence of SEQ ID NO: 63; and, (6b) a light chain variable region (LCVR), comprising a LCVR CDR1 sequence of SEQ ID NO: 64, a LCVR CDR2 sequence of SEQ ID NO: 65, and a LCVR CDR3 sequence of SEQ ID NO: 66; or (7a) a heavy chain variable region (HCVR), comprising a HCVR CDR1 sequence of SEQ ID NO: 71, a HCVR CDR2 sequence of SEQ ID NO: 72, and a HCVR CDR3 sequence of SEQ ID NO: 73; and, (7b) a light chain variable region (LCVR), comprising a LCVR CDR1 sequence of SEQ ID NO: 74, a LCVR CDR2 sequence of SEQ ID NO: 75, and a LCVR CDR3 sequence of SEQ ID NO: 76; or (8a) a heavy chain variable region (HCVR), comprising a HCVR CDR1 sequence of SEQ ID NO: 81, a HCVR CDR2 sequence of SEQ ID NO: 82, and a HCVR CDR3 sequence of SEQ ID NO: 83; and, (8b) a light chain variable region (LCVR), comprising a LCVR CDR1 sequence of SEQ ID NO: 84, a LCVR CDR2 sequence of SEQ ID NO: 85, and a LCVR CDR3 sequence of SEQ ID NO: 86; or (9a) a heavy chain variable region (HCVR), comprising a HCVR CDR1 sequence of SEQ ID NO: 91, a HCVR CDR2 sequence of SEQ ID NO: 92, and a HCVR CDR3 sequence of SEQ ID NO: 93; and, (9b) a light chain variable region (LCVR), comprising a LCVR CDR1 sequence of SEQ ID NO: 94, a LCVR CDR2 sequence of SEQ ID NO: 95, and a LCVR CDR3 sequence of SEQ ID NO: 96; optionally, said isolated monoclonal antibody is not naturally occurring; and/or, optionally further comprising a signal peptide sequence of SEQ ID NO: 41 at the N-terminus of said HCVR and/or LCVR.

In any of the proceding embodiments, in the isolated or recombinantly produced monoclonal antibody or antigen-binding fragment thereof: (1A) the HCVR sequence is SEQ ID NO: 7; and/or, (1B) the LCVR sequence is SEQ ID NO: 8, or, (2A) the HCVR sequence is SEQ ID NO: 17; and/or, (2B) the LCVR sequence is SEQ ID NO: 18, or, (3A) the HCVR sequence is SEQ ID NO: 27; and/or, (3B) the LCVR sequence is SEQ ID NO: 28, or, (4A) the HCVR sequence is SEQ ID NO: 37; and/or, (4B) the LCVR sequence is SEQ ID NO: 38 or SEQ ID NO: 114; (5A) the HCVR sequence is SEQ ID NO: 57; and/or, (5B) the LCVR sequence is SEQ ID NO: 58, or, (6A) the HCVR sequence is SEQ ID NO: 67; and/or, (6B) the LCVR sequence is SEQ ID NO: 68, or, (7A) the HCVR sequence is SEQ ID NO: 77; and/or, (7B) the LCVR sequence is SEQ ID NO: 78, or, (8A) the HCVR sequence is SEQ ID NO: 87; and/or, (8B) the LCVR sequence is SEQ ID NO: 88, or, (9A) the HCVR sequence is SEQ ID NO: 97; and/or, (9B) the LCVR sequence is SEQ ID NO: 98.

In any of the proceding embodiments, the monoclonal antibody has: (1a) a heavy chain sequence of SEQ ID NO: 9; and/or, (1b) a light chain sequence of SEQ ID NO: 10, or, (2a) a heavy chain sequence of SEQ ID NO: 19; and/or, (2b) a light chain sequence of SEQ ID NO: 20, or, (3a) a heavy chain sequence of SEQ ID NO: 29; and/or, (3b) a light chain sequence of SEQ ID NO: 30, or, (4a) a heavy chain sequence of SEQ ID NO: 39; and/or, (4b) a light chain sequence of SEQ ID NO: 40; (5a) a heavy chain sequence of SEQ ID NO: 59; and/or, (5b) a light chain sequence of SEQ ID NO: 60, or, (6a) a heavy chain sequence of SEQ ID NO: 69; and/or, (6b) a light chain sequence of SEQ ID NO: 70, or, (7a) a heavy chain sequence of SEQ ID NO: 79; and/or, (7b) a light chain sequence of SEQ ID NO: 80, or, (8a) a heavy chain sequence of SEQ ID NO: 89; and/or, (8b) a light chain sequence of SEQ ID NO: 90, or, (9a) a heavy chain sequence of SEQ ID NO: 99; and/or, (9b) a light chain sequence of SEQ ID NO: 100.

In any of the proceding embodiments, the monoclonal antibody has: (1a) a heavy chain sequence of SEQ ID NO: 101; and/or, (1b) a light chain sequence of SEQ ID NO: 10, or, (2a) a heavy chain sequence of SEQ ID NO: 102; and/or, (2b) a light chain sequence of SEQ ID NO: 20, or, (3a) a heavy chain sequence of SEQ ID NO: 103; and/or, (3b) a light chain sequence of SEQ ID NO: 30, or, (4a) a heavy chain sequence of SEQ ID NO: 104; and/or, (4b) a light chain sequence of SEQ ID NO: 40 or SEQ ID NO: 113.

In any of the proceding embodiments, the isolated or recombinantly produced monoclonal antibody or antigen-binding fragment thereof is a human antibody, a CDR-grafted antibody, or a resurfaced antibody.

In any of the proceding embodiments, the isolated or recombinantly produced monoclonal antibody or antigen-binding fragment thereof is a human IgG4 antibody, or an FcγR null monoclonal antibody engineered to prevent FcγR engagement.

In any of the proceding embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In any of the proceding embodiments, the monoclonal antibody or antigen-binding fragment thereof binds to the S1 glycoprotein of SARS-CoV-2.

In any of the proceding embodiments, the monoclonal antibody or antigen-binding fragment thereof binds to the S2 glycoprotein of SARS-CoV-2.

In any of the proceding embodiments, the isolated or recombinantly produced monoclonal antibody or antigen-binding fragment thereof binds to SARS-CoV-2 wild-type S protein and/or RBD/S1 variants selected from the group consisting of S477N, S494P, F490S, Y453F, N439K, N501Y, E484K, Q493R, and A222V/D614G.

In any of the proceding embodiments, the monoclonal antibody or antigen-binding fragment thereof binds the SARS-CoV-2 antigen with a K_{d} of less than about 10 nM, 5 nM, 2 nM, 1 nM, 0.5 nM, 0.2 nM, 0.1 nM, or 0.05 nM.

In any of the proceding embodiments, the isolated or recombinantly produced monoclonal antibody or antigen-binding fragment thereof inhibits binding of the SARS-CoV-2 antigen (e.g., the S1 glycoprotein) to ACE2.

In any of the proceding embodiments, the isolated or recombinantly produced monoclonal antibody or antigen-binding fragment thereof inhibits binding of the SARS-CoV-2 antigen (e.g., the S1 glycoprotein) to ACE2 immobilized on a solid support (such as in ELISA assay), or inhibits binding of the SARS-CoV-2 antigen (e.g., the S1 glycoprotein) to ACE2 expressed on the surface of a cell (such as Vero E6 cell).

In any of the proceding embodiments, the isolated or recombinantly produced monoclonal antibody or antigen-binding fragment thereof inhibits binding of the SARS-CoV-2 antigen (e.g., the S1 glycoprotein) to ACE2 with an EC50 value of less than 2 nM, 1 nM or 0.1 nM.

In any of the proceding embodiments, the isolated or recombinantly produced monoclonal antibody or antigen-binding fragment thereof exhibits neutralizing activity against a pseudovirus of SARS-CoV-2 or a live SARS-CoV-2 virus with an IC50 value of less than 10 nM, 8 nM, 6 nM, 5 nM, 3 nM, 2 nM, 1 nM, 0.6 nM or less than 0.5 nM.

In any of the proceding embodiments, the isolated or recombinantly produced monoclonal antibody or antigen-binding fragment thereof inhibits SARS-CoV-2 viral entry of a target cell (such as Vero E6 cell) at less than 10 nM, less than 5 nM, less than 2 nM, less than 1 nM, less than 0.5 nM, less than 0.2 nM, less than 0.1 nM, less than 0.08 nM, less than 0.06 nM, less than 0.02 nM, or less than 0.01 nM.

In any of the proceding embodiments, the isolated or recombinantly produced monoclonal antibody or antigen-binding fragment thereof of inhibits SARS-CoV-2 viral entry of a target cell (such as Vero E6 cell) with an IC50 of less than 10 nM, less than 5 nM, less than 3 nM, less than 2 nM, less than 1 nM, less than 500 pM, less than 300 pM, less than 200 pM, less than 100 pM, less than 80 pM, less than 50 pM, less than 30 pM, less than 10 pM, or less than 5 pM.

In any of the proceding embodiments, the isolated monoclonal antibody or antigen-binding fragment thereof inhibits entry of wild-type SARS-CoV-2 and/or SARS-CoV-2 variants, e.g. WuhanD614, BavPat D614G, UK B.1.1.7, or South Africa B.1.351 lineage, into a target cell.

In any of the proceding embodiments, the isolated monoclonal antibody or antigen-binding fragment thereof inhibits entry of a SARS-CoV-2 variant sharing one or more S protein mutations with the WuhanD614, BavPat D614G, UK B.1.1.7, and/or South Africa B.1.351 strain(s), into a target cell.

In any of the proceding embodiments, the isolated monoclonal antibody or antigen-binding fragment thereof does not cause antibody-dependent enhancement (ADE).

In any of the proceding embodiments, the isolated or recombinantly produced monoclonal antibody or antigen-binding fragment thereof comprises a heavy chain constant region, wherein the heavy chain constant region is human IgG1, human IgG2, human IgG3 or human IgG4.

In any of the proceding embodiments, the isolated or recombinantly produced monoclonal antibody or antigen-binding fragment thereof comprises a heavy chain constant region, wherein the heavy chain constant region is human IgG4.

In any of the proceding embodiments, the isolated or recombinantly produced monoclonal antibody or antigen-binding fragment thereof comprises a heavy chain variable region (HCVR), comprising a HCVR CDR1 sequence of SEQ ID NO: 11, a HCVR CDR2 sequence of SEQ ID NO: 12, and a HCVR CDR3 sequence of SEQ ID NO: 13, and a light chain variable region (LCVR), comprising a LCVR CDR1 sequence of SEQ ID NO: 14, a LCVR CDR2 sequence of SEQ ID NO: 15, and a LCVR CDR3 sequence of SEQ ID NO: 16.

In any of the proceding embodiments, the isolated or recombinantly produced monoclonal antibody or antigen-binding fragment thereof comprises HCVR sequence of SEQ ID NO: 17 or having at least 90%, 91%, 92%, 93%, 94%, 95%, 96%, 97%, 98%, 99% sequence identity to SEQ ID NO: 17, and an LCVR sequence of SEQ ID NO: 18 or having at least 90%, 91%, 92%, 93%, 94%, 95%, 96%, 97%, 98%, 99% sequence identity to SEQ ID NO: 18.

In any of the proceding embodiments, the isolated or recombinantly produced monoclonal antibody or antigen-binding fragment thereof comprises a heavy chain constant region, wherein the heavy chain constant region is human IgG1, human IgG2, human IgG3 or human IgG4. In some embodiments, the heavy chain constant region is human IgG4.

In any of the proceding embodiments, the isolated or recombinantly produced monoclonal antibody or antigen-binding fragment thereof comprises an HC sequence of SEQ ID NO: 19 or having at least 90%, 91%, 92%, 93%, 94%, 95%, 96%, 97%, 98%, 99% sequence identity to SEQ ID NO: 19.

In any of the proceding embodiments, the isolated or recombinantly produced monoclonal antibody or antigen-binding fragment thereof comprises an HC sequence of SEQ ID NO: 102 or having at least 90%, 91%, 92%, 93%, 94%, 95%, 96%, 97%, 98%, 99% sequence identity to SEQ ID NO: 102.

Another aspect of the invention provides an isolated or recombinantly produced monoclonal antibody or an antigen-binding fragment thereof, which competes with the isolated monoclonal antibody or antigen-binding fragment thereof of for binding to the same epitope.

In a related aspect, the invention provides a non-naturally occurring or existing therapeutic antibody based on the antigen-binding sequences of an antibody isolated from the patient using the method of the invention. Such therapeutic antibody may share one or more CDRs, such as CDR1, CDR2, and/or CDR3 of heavy chain and/or light chain sequences, with the antibody isolated from the patient using the method of the invention.

For example, heavy chain CDR3 (HC-CDR3) sequences of certain isolated antibodies are listed in Example 1.

Another aspect of the invention provides a mixture of two or more isolated or recombinantly produced monoclonal antibody or antigen-binding fragment thereof of the invention.

In certain embodiments, the proportion of each of said two or more isolated or recombinantly produced monoclonal antibody or antigen-binding fragment thereof is substantially the same, or is different.

Another aspect of the invention provides a method of treating or preventing a disease or condition arising from SARS-CoV-2 infection, the method comprising administering to a patient in need thereof an effective amount of the isolated or recombinantly produced monoclonal antibody or antigen-binding fragment thereof of the invention, or an effective amount of the mixture of the invention.

In certain embodiments, the method is for treating COVID-19, wherein the method further comprises administering a second therapeutic agent, which may be effective to treat infection by SARS-CoV-2.

In certain embodiments, the second therapeutic agent comprises chloroquine or hydroxychloroquine, remdesivir, lopinavir and ritonavir, azithromycin, an immune system inhibitor to inhibits cytokine storm (such as an anti-IL-6 neutralizing antibody such as tocilizumab or sarilumab), CD24Fc, IFX-1, an anti-CCR5 antibody such as Leronlimab, DAS181, CM4620, an anti-IFNγ monoclonal antibody such as emapalumab, an IL-1R antagonist such as Anakinra, Danoprevir+Ritonavir, Calquence (acalabrutinib), Xeljanz (tofacitinib), Jakafi (ruxolitinib), Olumiant (baricitinib), Ilaris (canakinumab), Otezla (apremilast), Mavrilimumab, or combination thereof.

In certain embodiments, the second therapeutic agent comprises one or more of: an anti-viral agent, an antibiotic, an anti-inflammatory agent or DMARD (disease-modifying anti-rheumatic drug).

Another aspect of the invention provides a polynucleotide encoding the heavy chain or the light chain or the antigen-binding portion thereof of the invention.

In certain embodiments, the polynucleotide is codon optimized for expression in a human cell.

Another aspect of the invention provides a vector comprising the polynucleotide of the invention.

In certain embodiments, the vector is an expression vector (e.g., a mammalian expression vector, a yeast expression vector, an insect expression vector, or a bacterial expression vector).

Another aspect of the invention provides a host cell comprising the vector of the invention, which expresses said isolated or recombinantly produced monoclonal antibody or antigen-binding fragment thereof.

Another aspect of the invention provides a pharmaceutical composition comprising the isolated or recombinantly produced monoclonal antibody or antigen-binding fragment thereof of the invention, or the mixture of the invention. The pharmaceutical composition further comprises a pharmaceutically acceptable excipient or diluent.

In certain embodiments, the pharmaceutical composition is formulated for intravenous administration.

It should be understood that any one embodiment of the invention, including those only described under one aspect or section of the invention, and those only described in the examples or claims, can be combined with any other embodiment(s) of the invention unless improper or expressly disclaimed.

### BRIEF DESCRIPTION OF THE DRAWINGS

FIG. 1 shows lead antibody binding characteristics in terms of binding to SARS-CoV-2 S antigen.

FIG. 2A shows the blocking activities of the subject antibodies, based on FACS analysis of blocked S1 binding to ACE2, using Vero E6 cell line expressing ACE2.

FIG. 2B shows potent neutralizing activities of the subject antibodies for neutralization of pseudovirus entry.

FIG. 3A shows the abilities of the subject antibodies to neutralize live SARS-CoV-2 cell entry. Using fluorescent labeled nuclear protein of SAR-CoV-2, infected cells can be observed with a florescence microscope. The top, middle, and bottom panels are for Ab-2, Ab-3, and Ab-1, respectively.

FIG. 3B shows dose response curves for live virus assay performed with three of the subject antibodies. The left, middle, and right panels are for Ab-2, Ab-1, and Ab-3, respectively.

FIG. 4A shows binding to full-length S protein (left panel) and inhibition of S1 protein binding to hACE2 (right panel) by Ab-2 (C1S5-2A2A) and variants thereof (C1S5-2A2A-1, -2, -3, -4, -9, -10, -11, -14, -15, -18, and -19).

FIG. 4B shows sequence alignments of Ab-2 and variants thereof.

FIG. 5 shows the PK profiles of the subject neutralizing antibodies in mice.

FIG. 6 shows binding of the subject antibodies to full-length S protein. The top to bottom labels are for Ab-2, -4, -3, -1, -6, -7, -5, -8, and -9, respectively.

FIG. 7 shows comparable binding activity to S1 RBD protein by IgG1 and IgG4 formats of Ab-2 based on ELISA. The lower EC50 value by the IgG1 format is likely due to the different secondary antibodies used in the assays.

FIG. 8 shows comparable pseudo-virus neutralization activity by IgG1 vs IgG4 formats of Ab-2.

FIG. 9 shows oropharyngeal viral loads (measured by RT-qPCR method) in Rhesus monkeys administered with control IgG4 antibody at 50 mg/kg (animal code C1 to C3) or Ab-2 IgG4 at 10 mg/kg (animal code 10 mg-1 to 10 mg-3) or at 50 mg/kg (animal code 50 mg-1 to 50 mg-3) on the day after intratracheal inoculation (1 day post infection [1 dpi]) with SARS-CoV-2 (1×10⁵ TCID₅₀/animal). Dotted line: detection threshold (Limit of Detection [L.O.D.]), 200 copies/mL.

FIGS. 10A-10C show viral loads (measured by RT-qPCR method) in the trachea, right bronchus, left bronchus, and different lobes of the lungs of Rhesus monkeys administered control IgG4 at 50 mg/kg, or Ab-2 IgG4 at 10 mg/kg or 50 mg/kg on day 5 (FIG. 10A, animals C1, AC1, and AC4, 5 dpi), day 6 (FIG. 10B, animals C3, AC3, and AC6, 6 dpi), and day 7 (FIG. 10C, animals C2, AC2, and AC5, 7 dpi) after intratracheal inoculation with SARS-CoV-2 (1×10⁵ TCID₅₀/animal). Control IgG4 or Ab-2 IgG4 were administered 1 day post inoculation (1 dpi). Dotted lines and grey area: detection threshold, 1000-10,000 copies/g.

FIG. 11 shows the partial sequence of the SARS-CoV-2 Spike (S) protein including residues that may participate in ACE2 and/or Ab-2 binding.

### DETAILED DESCRIPTION OF THE INVENTION

### 1. Overview

One aspect of the invention provides an antibody isolated from a convalescent COVID-19 patient using the method of the invention. Specifically, sera from convalescent COVID-19 (i.e., SARS-CoV-2) patients, a source of antiviral antibodies capable of conferring protective immunity on recipients, were obtained to identify effective antibodies against antigens of COVID-19 for therapeutic purposes. Antibodies identified from patients infected with the Ebola virus have been used as therapeutic antibodies (Bornholdt et al., 2016; Casadevall & Pirofski, 2020).

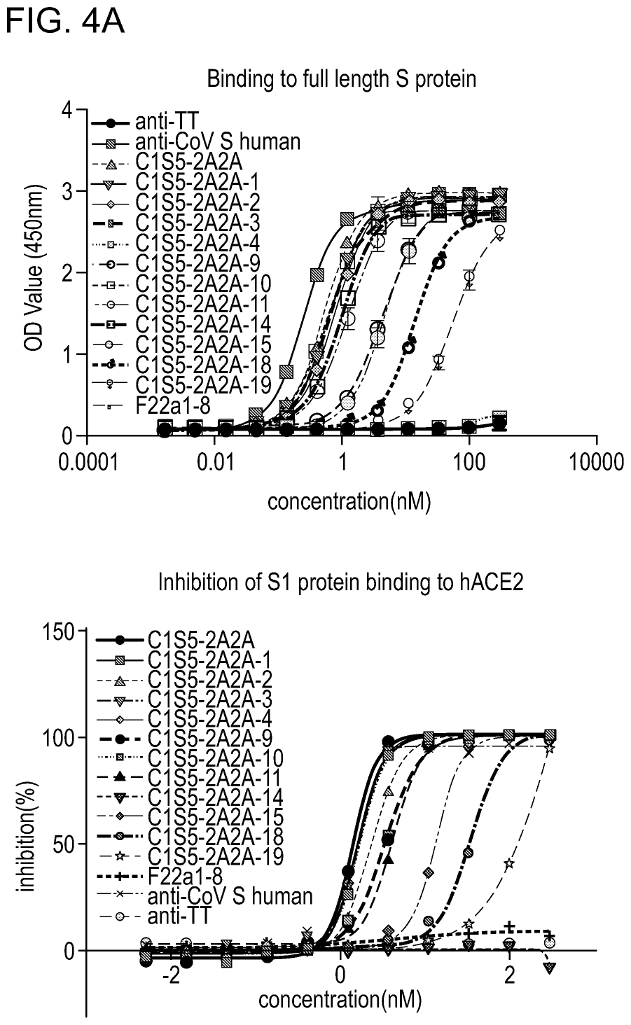

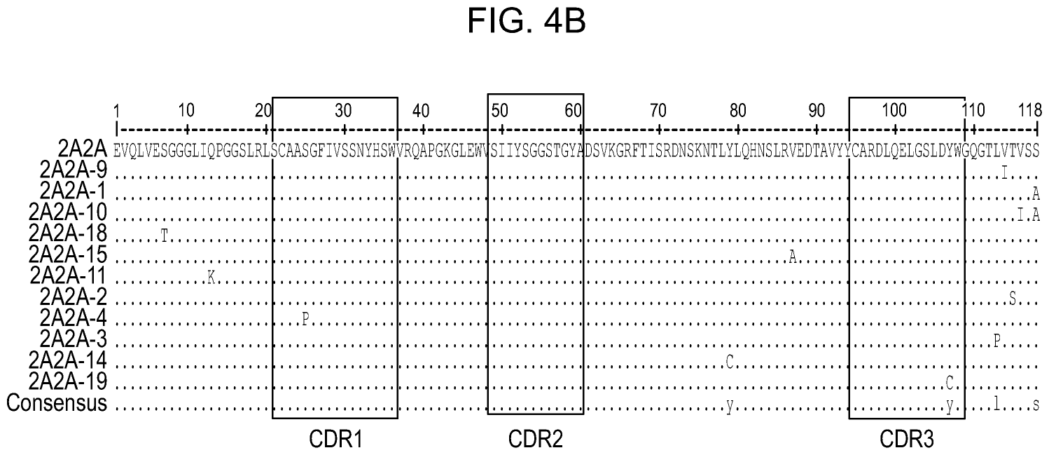

In a related aspect, the invention provides a non-naturally occurring or existing therapeutic antibody based on the antigen-binding sequences of an antibody isolated from the patient using the method of the invention. Such therapeutic antibody may share one or more CDRs, such as CDR1, CDR2, and/or CDR3 of heavy chain and/or light chain sequences, with the antibody isolated from the patient using the method of the invention. For example, heavy chain CDR3 (HC-CDR3) sequences of certain isolated antibodies are listed in FIG. 4B, or any of the CDR sequences or combinations thereof disclosed herein. Such antibodies may also be multi-specific (e.g., bi-specific), having antigen binding sequences originating from different antibody light and/or heavy chains.

Another aspect of the invention provides a mixture of the antibodies of the invention. Such a mixture may provide better therapeutic efficacy compared to the individual component antibodies of the mixture.

Indeed, a convalescent patient's sera is a natural mixture of different antibodies against the same or different viral antigen or epitope. Further, Applicant has identified multiple antibodies from the serum of convalescent patients, including 10 antibodies capable of binding to the full length SARS-CoV-2 S protein, among which 8 antibodies recognizes S1 only, and 2 antibodies interacts with S2 only but not with S1. The data indicates that diverse epitopes on SARS-CoV-2 virus spike protein can be targeted by different antibodies, suggesting that the antibodies of the invention, either alone or in combination with antibodies that target different epitopes via different mechanisms, may serve as potent therapeutic agents to treat COVID-19 patients.

Another aspect of the invention provides a polynucleotide encoding the heavy or light chain of the antibodies of the invention. Such polynucleotide sequences may be codon optimized for expression in a host cell, such as a mammalian cell line (e.g., CHO cell line) for large scale production of antibody.

Another aspect of the invention provides a vector comprising the polynucleotide of the invention. Such vector may be used for expression of antibody in a suitable host cell.

A further aspect of the invention provides a host cell comprising the vector of the invention, or producing the antibody of the invention.

Yet another aspect of the invention provides a method of treating or preventing a disease or condition arising from SARS-CoV-2 infection, such as COVID-19, the method comprising administering to a patient in need thereof a therapeutically effective amount of the antibody of the invention, or a mixture thereof.

With the general aspects of the inventions described, the following sections provide more detailed aspects of the invention. It should be understood that any one embodiment of the invention, including those only described in one section or one example, can be combined with any one or more additional embodiment of the invention whenever proper.

### 2. Definitions

The term “antibody,” in the broadest sense, encompasses various antibody structures, including but not limited to monoclonal antibodies, polyclonal antibodies, and multispecific antibodies (e.g., bispecific antibodies). The term “antibody” may also broadly refers to a molecule comprising complementarity determining region (CDR) 1, CDR2, and CDR3 of a heavy chain and CDR1, CDR2, and CDR3 of a light chain, wherein the molecule is capable of binding to an antigen. The term “antibody” also includes, but is not limited to, chimeric antibodies, humanized antibodies, human antibodies, and antibodies of various species such as mouse, human, cynomolgus monkey, etc.

In a narrower sense, however, “antibody” refers to the various monoclonal antibodies, including chimeric monoclonal antibodies, humanized monoclonal antibodies, and human monoclonal antibodies, particularly humanized or human monoclonal antibodies of the invention.

In some embodiments, an antibody comprises a heavy chain variable region (HCVR) and a light chain variable region (LCVR). In some embodiments, an antibody comprises at least one heavy chain (HC) comprising a heavy chain variable region and at least a portion of a heavy chain constant region, and at least one light chain (LC) comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region.

As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

The term “heavy chain variable region (HCVR)” as used herein refers to, at a minimum, a region comprising heavy chain CDR1 (CDR-H1), framework 2 (HFR2), CDR2 (CDR-H2), FR3 (HFR3), and CDR3 (CDR-H3). In some embodiments, a heavy chain variable region also comprises at least a portion (e.g., the whole) of an FR1 (HFR1), which is N-terminal to CDR-H1, and/or at least a portion (e.g., the whole) of an FR4 (HFR4), which is C-terminal to CDR-H3.

The term “heavy chain constant region” as used herein refers to a region comprising at least three heavy chain constant domains, CH1, CH2, and CH3. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising an F constant region is an IgE antibody, and an antibody comprising an μ constant region is an IgM antibody.

Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an α1 constant region) and IgA2 (comprising an α2 constant region) antibodies; and IgM antibodies include, but are not limited to, IgM1 (comprising an 1 constant region) and IgM2 (comprising μ2 constant region).

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence, and with or without a C-terminal lysine.

The term “light chain variable region (LCVR)” as used herein refers to a region comprising light chain CDR1 (CDR-L1), framework (FR) 2 (LFR2), CDR2 (CDR-L2), FR3 (LFR3), and CDR3 (CDR-L3). In some embodiments, a light chain variable region also comprises at least a portion (e.g., the whole) of an FR1 (LFR1) and/or at least a portion (e.g., the whole) of an FR4 (LFR4).

The term “light chain constant region” as used herein refers to a region comprising a light chain constant domain, C_{L}. Non-limiting exemplary light chain constant regions include λ and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The term “antibody fragment” or “antigen binding portion” (of antibody) includes, but is not limited to, fragments that are capable of binding antigen, such as Fv, single-chain Fv (scFv), Fab, Fab′, and (Fab′)₂. In certain embodiments, an antibody fragment includes Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

The term “Fab” refers to an antibody fragment with a molecular mass of approximately 50,000 Daltons, and has an activity of binding to the antigen. It comprises approximately half of the N-terminal side of the heavy chain and the whole of the light chain connected by a disulfide bridge. The Fab can be obtained in particular by treatment of immunoglobulin by a protease, papain.

The term “F(ab′)₂” designates a fragment of approximately 100,000 Daltons and an activity of binding to the antigen. This fragment is slightly larger than two Fab fragments connected via a disulfide bridge in the hinge region. These fragments are obtained by treating an immunoglobulin with a protease, pepsin. The Fab fragment can be obtained from the F(ab′)2 fragment by cleaving of the disulfide bridge of the hinge region.

A single Fv chain “scFv” corresponds to a VH: VL polypeptide synthesized using the genes coding for the VL and VH domains and a sequence coding for a peptide intended to bind these domains. An scFv according to the invention includes the CDRs maintained in an appropriate conformation, for example using genetic recombination techniques.

The dimers of “scFv” correspond to two scFv molecules connected together by a peptide bond. This Fv chain is frequently the result of the expression of a fusion gene including the genes coding for VH and VL connected by a linker sequence coding a peptide. The human scFv fragment may include CDR regions that are maintained in an appropriate conformation, preferably by means of the use of genetic recombination techniques.

The “dsFv” fragment is a VH-VL heterodimer stabilized by a disulfide bridge; it may be divalent (dsFV₂). Fragments of divalent Sc(Fv)₂ or multivalent antibodies may form spontaneously by the association of monovalent scFvs or be produced by connecting scFvs fragments by peptide binding sequences.

The Fc fragment is the support for the biological properties of the antibody, in particular its ability to be recognized by immunity effectors or to activate the complement. It consists of constant fragments of the heavy chains beyond the hinge region.

The term “diabodies” signifies small antibody fragments having two antigen fixing sites. These fragments comprise, in the same VH-VL polypeptide chain, a variable heavy chain domain VH connected to a variable light chain domain VL. Using a binding sequence that is too short to allow the matching of two domains of the same chain, the matching with two complementary domains of another chain necessarily occurs and thus two antigen fixing sites are created.

An “antibody that binds to the same epitope” as a reference antibody can be determined by an antibody competition assay. It refers to an antibody that blocks binding of the reference antibody to its antigen in a competition assay by 50% or more, and conversely, the reference antibody blocks binding of the antibody to its antigen in a competition assay by 50% or more. The term “compete” when used in the context of an antibody that compete for the same epitope means competition between antibodies is determined by an assay in which an antibody being tested prevents or inhibits specific binding of a reference antibody to a common antigen.

Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using I¹²⁵ label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol.).

Typically, such an assay involves the use of purified antigen bound to a solid surface or cells bearing either of these, an unlabeled test antigen binding protein and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibodies and antibodies binding to an adjacent epitope sufficiently proximal to the epitope bound by the reference antibody for steric hindrance to occur. In some embodiments, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more.

The term “antigen” refers to a molecule or a portion of a molecule capable of being bound by a selective binding agent, such as an antibody or immunologically functional fragment thereof, and additionally capable of being used in a mammal to produce antibodies capable of binding to that antigen. An antigen may possess one or more epitopes that are capable of interacting with antibodies.

The term “epitope” is the portion of an antigen molecule that is bound by a selective binding agent, such as an antibody or a fragment thereof. The term includes any determinant capable of specifically binding to an antibody. An epitope can be contiguous or non-contiguous (e.g., in a polypeptide, amino acid residues that are not contiguous to one another in the polypeptide sequence but that within in context of the molecule are bound by the antigen binding protein). In some embodiments, epitopes may be mimetic in that they comprise a three dimensional structure that is similar to an epitope used to generate the antibody, yet comprise none or only some of the amino acid residues found in that epitope used to generate the antibody. Epitope determinants may include chemically active surface groupings of molecules such as amino acids, sugar side chains, phosphoryl or sulfonyl groups, and may have specific three dimensional structural characteristics, and/or specific charge characteristics.

In some embodiments, an “epitope” is defined by the method used to determine it. For example, in some embodiments, an antibody binds to the same epitope as a reference antibody, if they bind to the same region of the antigen, as determined by hydrogen-deuterium exchange (HDX).

In certain embodiments, an antibody binds to the same epitope as a reference antibody if they bind to the same region of the antigen, as determined by X-ray crystallography.

A “human antibody” as used herein refers to antibodies of human origin or antibodies produced in humans, antibodies produced in non-human animals that comprise human immunoglobulin genes, such as XENOMOUSE®, and antibodies selected using in vitro methods, such as phage display, wherein the antibody repertoire is based on a human immunoglobulin sequences.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Non-limiting exemplary mammalian cells include, but are not limited to, NSO cells, PER.C6® cells (Crucell), and 293 and CHO cells, and their derivatives, such as 293-6E and DG44 cells, respectively.

The term “isolated” as used herein refers to a molecule that has been separated from at least some of the components with which it is typically found in nature or has been separated from at least some of the components with which it is typically produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide.

In certain embodiments, an isolated antibody of the invention may have natural human antibody sequence, but is so purified that it consists essentially of the antibody, such as a monoclonal antibody recombinantly produced and isolated/purified from the cells which produce such antibody.

In certain embodiments, the isolated antibody is at least 90% pure, 95% pure, 97% pure, 99% pure, 99.5% pure, 99.9% pure or purer.

Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a mammal such as human. In some embodiments, methods of treating other non-human mammals, including, but not limited to, rodents, simians, felines, canines, equines, bovines, porcines, ovines, caprines, mammalian laboratory animals, mammalian farm animals, mammalian sport animals, and mammalian pets, are also provided. In some instances, a “subject” or “patient” refers to a (human) subject or patient in need of treatment for a disease or disorder.

The term “sample” or “patient sample” as used herein, refers to material that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample,” “reference cell,” or “reference tissue,” as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of at least one individual who is not the subject or patient in whom a disease or condition is being identified using a composition or method of the invention. In some embodiments, a reference sample, reference cell or reference tissue was previously obtained from a patient prior to developing a disease or condition or at an earlier stage of the disease or condition.

A “disorder” or “disease” is any condition that would benefit from treatment with one or more antibodies of the invention. This includes COVID-19 or any secondary infection by other bacteria or virus, in which the antibody of the invention is used in a combination therapy.

The term “antibody-dependent enhancement” (ADE) refers to a phenomenon in which binding of a virus to suboptimal antibodies enhances its entry into host cells. While not wishing to be bound by any particular theory and not implying that any actual mechanisms of ADE may have been operating in connection with any antibodies tested herein, ADE has been documented to occur through two distinct mechanisms in viral infections: by enhanced antibody-mediated virus uptake into Fc gamma receptor IIa (FcγRIIa)-expressing phagocytic cells leading to increased viral infection and replication, or by excessive antibody Fc-mediated effector functions or immune complex formation causing enhanced inflammation and immunopathology. Both ADE pathways can occur when non-neutralizing antibodies or antibodies at sub-neutralizing levels bind to viral antigens without blocking or clearing infection.

“Treatment” refers to therapeutic treatment, for example, wherein the object is to slow down (lessen) the targeted pathologic condition or disorder as well as, for example, wherein the object is to inhibit recurrence of the condition or disorder. “Treatment” covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process. The term “treatment” also includes reducing the severity of any phenotypic characteristic and/or reducing the incidence, degree, or likelihood of that characteristic. Those in need of treatment include those already with the disorder as well as those at risk of recurrence of the disorder or those in whom a recurrence of the disorder is to be prevented or slowed down.

The term “effective amount” or “therapeutically effective amount” refers to an amount of a drug effective to treat a disease or disorder in a subject. In some embodiments, an effective amount refers to an amount effective, at dosages and for periods of time necessary, to achieve the desired therapeutic or prophylactic result. A therapeutically effective amount of antibody of the invention may vary according to factors such as the disease state, age, sex, and weight of the individual, and the ability of the antagonist to elicit a desired response in the individual. A therapeutically effective amount encompasses an amount in which any toxic or detrimental effects of subject antibody are outweighed by the therapeutically beneficial effects.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

An “article of manufacture” is any manufacture (e.g., a package or container) or kit comprising at least one reagent, e.g., a medicament for treatment of a disease or disorder, or a probe for specifically detecting a biomarker described herein. In some embodiments, the manufacture or kit is promoted, distributed, or sold as a unit for performing the methods described herein.

### 3. Routes of Administration and Carriers

In various embodiments, antibodies of the invention may be administered subcutaneously or intravenously.

In some embodiments, the subject antibody may be administered in vivo by various routes, including, but not limited to, oral, intra-arterial, parenteral, intranasal, intramuscular, intracardiac, intraventricular, intratracheal, buccal, rectal, intraperitoneal, by inhalation, intradermal, topical, transdermal, and intrathecal, or otherwise, e.g., by implantation.

In some embodiments, the subject antibody or antigen-binding fragment thereof is administered intraveneously (i.v.) or subcutaneously (s.c.).

The subject compositions may be formulated into preparations in solid, semi-solid, liquid, or gaseous forms; including, but not limited to, tablets, capsules, powders, granules, ointments, solutions, suppositories, enemas, injections, inhalants, and aerosols.

In various embodiments, compositions comprising the subject antibody are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Nonlimiting exemplary carriers include saline, buffered saline, dextrose, water, glycerol, ethanol, and combinations thereof.

In various embodiments, the subject antibody may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives.

In various embodiments, the compositions may be formulated for inhalation, for example, using pressurized acceptable propellants such as dichlorodifiuoromethane, propane, nitrogen, and the like.

The compositions may also be formulated, in various embodiments, into sustained release microcapsules, such as with biodegradable or non-biodegradable polymers. A non-limiting exemplary biodegradable formulation includes poly lactic acid-glycolic acid (PLGA) polymer. A non-limiting exemplary non-biodegradable formulation includes a polyglycerin fatty acid ester. Certain methods of making such formulations are described, for example, in EP 1125584 A1.

Pharmaceutical dosage packs comprising one or more containers, each containing one or more types or doses of the subject antibody, are also provided. In some embodiments, a unit dosage is provided wherein the unit dosage contains a predetermined amount of a composition comprising the subject antibody, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated.

In some embodiments, the subject antibody may be administered in an amount in the range of about 50 μg/kg body weight to about 50 mg/kg body weight per dose. In some embodiments, the subject antibody may be administered in an amount in the range of about 100 μg/kg body weight to about 50 mg/kg body weight per dose. In some embodiments, the subject antibody may be administered in an amount in the range of about 100 μg/kg body weight to about 20 mg/kg body weight per dose. In some embodiments, the subject antibody may be administered in an amount in the range of about 0.5 mg/kg body weight to about 20 mg/kg body weight per dose.

In some embodiments, the subject antibody may be administered in an amount in the range of about 10 mg to about 1,000 mg per dose. In some embodiments, the subject antibody may be administered in an amount in the range of about 20 mg to about 500 mg per dose. In some embodiments, the subject antibody may be administered in an amount in the range of about 20 mg to about 300 mg per dose. In some embodiments, the subject antibody may be administered in an amount in the range of about 20 mg to about 200 mg per dose.

The subject antibody compositions may be administered as needed to subjects. In some embodiments, an effective dose of the subject antibody is administered to a subject one or more times. In various embodiments, an effective dose of the subject antibody is administered to the subject once a day, less than once a week, such as, for example, every two days, every three days, or every six days. In other embodiments, an effective dose of the subject antibody is administered more than once a day, such as, for example, once or multiple times per day. An effective dose of the subject antibody is administered to the subject at least once. In some embodiments, the effective dose of the subject antibody may be administered multiple times, including for periods of at least a month, at least six months, or at least a year. In some embodiments, the subject antibody is administered to a subject as-needed to alleviate one or more symptoms of a condition.

### 4. Combination Therapy

The antibodies and functional fragments thereof of the invention may be administered to a subject in need thereof in combination with other biologically active substances or other treatment procedures for the treatment of diseases, e.g., COVID-19 and associated symptoms and/or complications. For example, the antibodies of the invention may be administered alone, together as a mixture or combination, or with other modes of treatment such as a second therapeutic agent effective to treat COVID-19 or symptoms/complications thereof. They may be provided before, substantially contemporaneous with, or after other modes of treatment.

In certain embodiments, the second therapeutic agent comprises one or more of: chloroquine or hydroxychloroquine, remdesivir, lopinavir and ritonavir, azithromycin, an immune system inhibitor to inhibits cytokine storm (such as an anti-IL-6 neutralizing antibody such as tocilizumab or sarilumab), CD24Fc, IFX-1, an anti-CCR5 antibody such as Leronlimab, DAS181, CM4620, an anti-IFNγ monoclonal antibody such as emapalumab, an IL-1R antagonist such as Anakinra, Danoprevir+Ritonavir, Calquence (acalabrutinib), Xeljanz (tofacitinib), Jakafi (ruxolitinib), Olumiant (baricitinib), Ilaris (canakinumab), Otezla (apremilast), Mavrilimumab, or combination thereof.

The administration of any two or more agents may start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

In certain aspects, the agents are administered simultaneously, e.g., are infused simultaneously, e.g., over a period of 30 or 60 minutes, to a patient.

### 5. Exemplary Antibodies

One aspect of the invention provides human antibodies that block binding of SARS-CoV-2 virus to a human cell receptor to gain viral entry of the human cell, such as inhibiting binding of the S1 glycoprotein to the ACE2 receptor.

In some embodiments, the antibody of the invention has a dissociation constant (K_{d}) of ≤1 μM, ≤100 nM, ≤10 nM, ≤5 nM, ≤2 nM, ≤1 nM, ≤0.5 nM, ≤0.2 nM, ≤0.1 nM, ≤0.05 nM, ≤0.01 nM, or ≤0.001 nM (e.g. 10−⁸ M or less, e.g. from 10⁻⁸ M to 10⁻¹³ M, e.g., from 10⁻⁹ M to 10⁻¹³ M) for the SARS-CoV-2, such as the S1 glycoprotein.

In some embodiments, the antibody of the invention inhibits binding of the SARS-CoV-2 antigen (e.g., the S1 glycoprotein) to ACE2. Such binding can be assessed in vitro using, for example, an ELISA assay using immobilized SARS-CoV-2 antigen on a solid support, or binding to a cell expressing ACE2 receptor on the surface.

In some embodiments, the antibody of the invention inhibits binding of the SARS-CoV-2 antigen (e.g., the S1 glycoprotein) to ACE2 with an EC50 value of less than 1 nM or 0.1 nM.

In some embodiments, the antibody of the invention exhibits neutralizing activity against a pseudovirus of SARS-CoV-2 or a live SARS-CoV-2 virus with an IC50 value of less than 10 nM, 6 nM, 3 nM, 2 nM, 1 nM, 0.6 nM or less than 0.5 nM.

In some embodiments, an antibody having any the characteristics provided herein inhibits at least 25%, 50%, 75%, 80%, 90% or 100% of the entry of SARS-CoV-2 into a host cell, such as according to the in vitro assay conditions used in the examples for entry into Vero E6 cells. Inhibition of live virus entry can be assayed based on the concentration of antibodies needed to protect about 50% SARS-CoV-2 susceptible cells, such as Vero E6 cells growing on monolayer, from exhibiting CPE (cytopathic effect) 3-5 days post infection (dpi).

In some embodiments, the antibody of the invention inhibits SARS-CoV-2 viral entry of a target cell (such as Vero E6 cell) at less than 10 nM, less than 5 nM, less than 2 nM, less than 1 nM, less than 0.5 nM, less than 0.2 nM, less than 0.1 nM, less than 0.08 nM, less than 0.06 nM, less than 0.02 nM, or less than 0.01 nM.

In some embodiments, the antibody of the invention inhibits SARS-CoV-2 viral entry of a target cell (such as Vero E6 cell) with an IC50 of less than 10 nM, 5 nM, 3 nM, 2 nM, 1 nM, 500 pM, 300 pM, 200 pM, 100 pM, 80 pM, 50 pM, 30 pM, 10 pM, or less than 5 pM.

In some embodiments, multispecific antibodies are provided. In some embodiments, bispecific antibodies are provided. Non-limiting exemplary bispecific antibodies include antibodies comprising a first arm comprising a heavy chain/light chain combination that binds a first epitope of SARS-CoV-2 and a second arm comprising a heavy chain/light chain combination that binds a second epitope of SARS-CoV-2. A further non-limiting exemplary multispecific antibody is a dual variable domain antibody.

In certain embodiments, the monoclonal antibodies of the invention or antigen-binding fragments thereof, including human monoclonal antibodies or antigen-binding fragments thereof, include one or more point mutations of in amino acid sequences that are designed to improve developability of the antibody. For example, Raybould et al. (Five computational developability guidelines for therapeutic antibody profiling, PNAS 116(10): 4025-4030, 2019) described Therapeutic Antibody Profiler (TAP), a computational tool that builds downloadable homology models of variable domain sequences, tests them against five developability guidelines, and reports potential sequence liabilities and canonical forms. The authors further provide TAP as freely available at opig.stats.ox.ac.uk/webapps/sabdab-sabpred/TAP.php.

There are many barriers to therapeutic mAb development, besides achieving the desired affinity to the antigen. These include intrinsic immunogenicity, chemical and conformational instability, self-association, high viscosity, polyspecificity, and poor expression. For example, high levels of hydrophobicity, particularly in the highly variable complementarity-determining regions (CDRs), have repeatedly been implicated in aggregation, viscosity, and polyspecificity. Asymmetry in the net charge of the heavy- and light-chain variable domains is also correlated with self-association and viscosity at high concentrations. Patches of positive and negative charge in the CDRs are linked to high rates of clearance and poor expression levels. Product heterogeneity (e.g., through oxidation, isomerization, or glycosylation) often results from specific sequence motifs liable to post- or co-translational modification. Computational tools are available to facilitate the identification of sequence liabilities. Warszawski et al. (Optimizing antibody affinity and stability by the automated design of the variable light-heavy chain interfaces. PLoS Comput Biol 15(8): e1007207. https://doi.org/10.1371/journal.pcbi.1007207) also described methods of optimizing antibody affinity and stability by an automated design of the variable light-heave chain interfaces. Additional methods are available to identify potential developability issues of a candidate antibody, and in preferred embodiments of this invention, one or more point mutations can be introduced, via conventional methods, to the candidate antibody to address such issues to lead to an optimized therapeutic antibody of the invention.

The sequences of certain representative antibodies, including the light chain (LC) and heavy chain (HC) variable regions, the CDR regions, and the framework regions (FR), are listed below.

### Ab-1

VH-CDR1:
(SEQ ID NO: 1)
CTVSGGSISSSIYYWGW
VH-CDR2:
(SEQ ID NO: 2)
GSIYYSGNAYYN
VH-CDR3:
(SEQ ID NO: 3)
CATPHTRWGPDYW
HCVR:
(SEQ ID NO: 7)
QLQLQESGPGLVKPSETLSLTCTVSGGSISSSIYYWGWIRQPPGKGLEWIGSIYYSGNAYYN
PSLKSRVTISVDTSKNQFSLKLNSVTAADTAVYYCATPHTRWGPDYWGQGTLVTVSS
Heavy chain sequence:
(SEQ ID NO: 9)
QLQLQESGPGLVKPSETLSLTCTVSGGSISSSIYYWGWIRQPPGKGLEWIGSIYYSGNAYYN
PSLKSRVTISVDTSKNQFSLKLNSVTAADTAVYYCATPHTRWGPDYWGQGTLVTVSSASTKG
PSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
HCVR-encoding polynucleotide sequence:
(SEQ ID NO: 42)
CAGCTGCAGCTGCAGGAGTCGGGCCCAGGACTGGTGAAGCCTTCGGAGACCCTGTCCCTCAC
CTGCACTGTCTCTGGTGGCTCCATCAGCAGTAGTATTTACTACTGGGGCTGGATCCGCCAGC
CCCCAGGGAAGGGGCTGGAGTGGATTGGGAGTATCTATTATAGTGGGAACGCCTACTATAAC
CCGTCCCTCAAGAGTCGAGTCACCATATCCGTGGACACGTCCAAGAACCAGTTCTCCCTGAA
GCTGAACTCTGTGACCGCCGCAGACACGGCTGTGTATTACTGTGCGACTCCGCACACACGGT
GGGGCCCTGACTACTGGGGCCAGGGAACCCTGGTCACCGTCTCCTCA
IgG4 Heavy chain sequence:
(SEQ ID NO: 101)
QLQLQESGPGLVKPSETLSLTCTVSGGSISSSIYYWGWIRQPPGKGLEWIGSIYYSGNAYYN
PSLKSRVTISVDTSKNQFSLKLNSVTAADTAVYYCATPHTRWGPDYWGQGTLVTVSSASTKG
PSVFPLAPCSRSTSESTAALGCLVKDYFPEPVTVSWNSGALTSGVHTFPAVLQSSGLYSLSS
VVTVPSSSLGTKTYTCNVDHKPSNTKVDKRVESKYGPPCPPCPAPEFLGGPSVFLFPPKPKD
TLYITREPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNH
YTQKSLSLSLGK
VL-CDR1:
(SEQ ID NO: 4)
CTRSSGSIASNYVLW
VL-CDR2:
(SEQ ID NO: 5)
EDDQRPS
VL-CDR3:
(SEQ ID NO: 6)
CQSYDGDNLVF
LCVR:
(SEQ ID NO: 8)
NFMLTQPHSVSASPGKTVTVSCTRSSGSIASNYVLWYQQRPGSAPTTVIYEDDQRPSGVPDR
FSASIDSSSNSASLTISGLKTEDEADYYCQSYDGDNLVFGGGTKLTVL
Light chain sequence:
(SEQ ID NO: 10)
NFMLTQPHSVSASPGKTVTVSCTRSSGSIASNYVLWYQQRPGSAPTTVIYEDDQRPSGVPDR
FSASIDSSSNSASLTISGLKTEDEADYYCQSYDGDNLVFGGGTKLTVLGQPKAAPSVTLFPP
SSEELQANKATLVCLISDFYPGAVTVAWKADSSPVKAGVETTTPSKQSNNKYAASSYLSLTP
EQWKSHRSYSCQVTHEGSTVEKTVAPTECS

For all the antibody heavy chain sequences, the framework region sequences HFR1-HFR4 are defined by the VH-CDR sequences. For example, HFR1 is the sequence of HCVR 30 that is N-terminal to VH-CDR1. HFR2 is the sequence of HCVR that is between VH-CDR1 and VH-CDR2. HFR3 is the sequence of HCVR that is between VH-CDR2 and VH-CDR3. HFR4 is the most C-terminal sequence of HCVR.

Likewise, for all the antibody light chain sequences, the framework region sequences LFR1-LFR4 are defined by the VL-CDR sequences. For example, LFR1 is the sequence of LCVR that is N-terminal to VL-CDR1. LFR2 is the sequence of LCVR that is between VL-CDR1 and VL-CDR2. LFR3 is the sequence of LCVR that is between VL-CDR2 and VL-CDR3. LFR4 is the most C-terminal sequence of LCVR.

### Ab-2

VH-CDR1:
(SEQ ID NO: 11)
CAASGFIVSSNYMSW
VH-CDR2:
(SEQ ID NO: 12)
SIIYSGGSTFYA
VH-CDR3:
(SEQ ID NO: 13)
CARDLQELGSLDYW
HCVR:
(SEQ ID NO: 17)
EVQLVESGGGLIQPGGSLRLSCAASGFIVSSNYMSWVRQAPGKGLEWVSIIYSGGSTFYADS
VKGRFTISRDNSKNTLYLQMNSLRVEDTAVYYCARDLQELGSLDYWGQGTLVTVSS
HC:
(SEQ ID NO: 19)
EVQLVESGGGLIQPGGSLRLSCAASGFIVSSNYMSWVRQAPGKGLEWVSIIYSGGSTFYADS
VKGRFTISRDNSKNTLYLQMNSLRVEDTAVYYCARDLQELGSLDYWGQGTLVTVSSASTKGP
SVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
HCVR-encoding polynucleotide sequence:
(SEQ ID NO: 43)
GAGGTGCAGCTGGTGGAGTCTGGAGGAGGCTTGATCCAGCCTGGGGGGTCCCTGAGACTCTC
CTGTGCAGCCTCTGGATTCATCGTCAGTAGCAACTACATGAGCTGGGTCCGCCAGGCTCCAG
GGAAGGGGCTGGAGTGGGTCTCAATTATTTATAGCGGTGGTAGTACATTCTACGCAGACTCC
GTGAAGGGCCGATTCACCATCTCCAGAGACAATTCCAAGAACACGCTGTATCTTCAAATGAA
CAGCCTGAGAGTCGAGGACACGGCCGTGTATTACTGTGCGAGAGATCTTCAGGAGCTCGGCT
CTCTTGACTACTGGGGCCAGGGAACCCTGGTCACCGTCTCCTCA
IgG4 Heavy chain sequence:
(SEQ ID NO: 102)
EVQLVESGGGLIQPGGSLRLSCAASGFIVSSNYMSWVRQAPGKGLEWVSIIYSGGSTFYADS
VKGRFTISRDNSKNTLYLQMNSLRVEDTAVYYCARDLQELGSLDYWGQGTLVTVSSASTKGP
SVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVFLFPPKPKDT
LYITREPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYP
SDIAVEWESNGQPENNYKTTPPVLDSDGSFFLYSRLTVDKSRWQEGNVFSCSVMHEALHNHY
TQKSLSLSLGK
VL-CDR1:
(SEQ ID NO: 14)
CRASQGISSWLAW
VL-CDR2:
(SEQ ID NO: 15)
AASSLQS
VL-CDR3:
(SEQ ID NO: 16)
CQEANSFPYTF
LCVR:
(SEQ ID NO: 18)
DIQMTQSPSSVSASVGDRVTITCRASQGISSWLAWYQQKPGKAPKLLIYAASSLQSGVPSRF
SGSGSGTDFTLTISSLQPEDFATYYCQEANSFPYTFGQGTKLEIK
LC:
(SEQ ID NO: 20)
DIQMTQSPSSVSASVGDRVTITCRASQGISSWLAWYQQKPGKAPKLLIYAASSLQSGVPSRF
SGSGSGTDFTLTISSLQPEDFATYYCQEANSFPYTFGQGTKLEIKRTVAAPSVFIFPPSDEQ
LKSGTASWCLLNNFYPREAKVQWKVDNALQSGNSQESVTEQDSKDSTYSLSSTLTLSKADY
EKHKVYACEVTHQGLSSPVTKSFNRGEC

### Ab-3

VH-CDR1:
(SEQ ID NO: 21)
CTVSGGSISSTIYYWGW
VH-CDR2:
(SEQ ID NO: 22)
GSIYYSGNAYYN
VH-CDR3:
(SEQ ID NO: 23)
CATPHTRWGPDYW
HCVR:
(SEQ ID NO: 27)
QLQLQESGPGLVKPSETLSLTCTVSGGSISSTIYYWGWIRQPPGKGLEWIGSIYYSGNAYYN
PSLKSRVTISVDTSKNQFSLMLNSVTASDTAVYYCATPHTRWGPDYWGQGTLVTVSS
HC:
(SEQ ID NO: 29)
QLQLQESGPGLVKPSETLSLTCTVSGGSISSTIYYWGWIRQPPGKGLEWIGSIYYSGNAYYN
PSLKSRVTISVDTSKNQFSLMLNSVTASDTAVYYCATPHTRWGPDYWGQGTLVTVSSASTKG
PSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
HCVR-encoding polynucleotide sequence:
(SEQ ID NO: 44)
CAGCTGCAGCTGCAGGAGTCGGGCCCAGGACTGGTGAAGCCTTCGGAGACCCTGTCCCTCAC
CTGCACTGTCTCTGGTGGCTCCATCAGCAGTACTATTTACTATTGGGGCTGGATCCGCCAGC
CCCCAGGGAAGGGGCTGGAGTGGATTGGGAGTATCTATTATAGTGGGAACGCCTACTATAAC
CCGTCCCTCAAGAGTCGAGTCACCATATCCGTGGACACGTCCAAGAACCAGTTCTCCCTGAT
GCTGAACTCTGTGACCGCCTCAGACACGGCTGTGTATTACTGTGCGACTCCGCACACACGGT
GGGGCCCTGACTACTGGGGCCAGGGAACCCTGGTCACCGTCTCCTCA
IgG4 Heavy chain sequence:
(SEQ ID NO: 103)
QLQLQESGPGLVKPSETLSLTCTVSGGSISSTIYYWGWIRQPPGKGLEWIGSIYYSGNAYYN
PSLKSRVTISVDTSKNQFSLMLNSVTASDTAVYYCATPHTRWGPDYWGQGTLVTVSSASTKG
PSVFPLAPCSRSTSESTAALGCLVKDYFPEPVTVSWNSGALTSGVHTFPAVLQSSGLYSLSS
VVTVPSSSLGTKTYTCNVDHKPSNTKVDKRVESKYGPPCPPCPAPEFLGGPSVFLFPPKPKD
TLYITREPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNH
YTQKSLSLSLGK
VL-CDR1:
(SEQ ID NO: 24)
CTRSSGSIASNYVLW
VL-CDR2:
(SEQ ID NO: 25)
EDDQRPS
VL-CDR3:
(SEQ ID NO: 26)
CQSYDGDNLVF
LCVR:
(SEQ ID NO: 28)
NFMLTQPHSVSASPGKTVTVSCTRSSGSIASNYVLWYQQRPGSAPTTVIYEDDQRPSGVPDR
FSASIDSSSNSASLTISGLKTEDEADYYCQSYDGDNLVFGGGTKLTVL
LC:
(SEQ ID NO: 30)
NFMLTQPHSVSASPGKTVTVSCTRSSGSIASNYVLWYQQRPGSAPTTVIYEDDQRPSGVPDR
FSASIDSSSNSASLTISGLKTEDEADYYCQSYDGDNLVFGGGTKLTVLGQPKAAPSVTLFPP
SSEELQANKATLVCLISDFYPGAVTVAWKADSSPVKAGVETTTPSKQSNNKYAASSYLSLTP
EQWKSHRSYSCQVTHEGSTVEKTVAPTECS

### Ab-4

VH-CDR1:
(SEQ ID NO: 31)
CTFSGFSLSTSGVGVGW
VH-CDR2:
(SEQ ID NO: 32)
ALIYWDDDKRYS
VH-CDR3:
(SEQ ID NO: 33)
CAHRLSNFWSGYYTGW
HCVR:
(SEQ ID NO: 37)
QITLKESGPTLVKPTQTLTLTCTFSGFSLSTSGVGVGWIRQPPGKALEWLALIYWDDDKRYS
PSLKSRLTITKDTSKNQVVLTMTNMAPVDTATYYCAHRLSNFWSGYYTGWGQGTLVTVSS
HC:
(SEQ ID NO: 39)
QITLKESGPTLVKPTQTLTLTCTFSGFSLSTSGVGVGWIRQPPGKALEWLALIYWDDDKRYS
PSLKSRLTITKDTSKNQVVLTMTNMAPVDTATYYCAHRLSNFWSGYYTGWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
HCVR-encoding polynucleotide sequence:
(SEQ ID NO: 45)
CAGATCACCTTGAAGGAGTCTGGTCCTACGCTGGTGAAACCCACACAGACCCTCACGCTGAC
CTGCACCTTCTCTGGGTTCTCACTCAGCACTAGTGGAGTGGGTGTGGGCTGGATCCGTCAGC
CCCCAGGAAAGGCCCTGGAGTGGCTTGCACTCATTTATTGGGATGATGATAAGCGCTACAGC
CCATCTCTGAAGAGCAGGCTCACCATCACCAAGGACACCTCCAAAAACCAGGTGGTCCTTAC
AATGACCAACATGGCCCCTGTGGACACAGCCACATATTACTGTGCACACAGACTCTCCAATT
TTTGGAGTGGTTATTATACTGGCTGGGGCCAGGGAACCCTGGTCACCGTCTCCTCA
IgG4 Heavy chain sequence:
(SEQ ID NO: 104)
QITLKESGPTLVKPTQTLTLTCTFSGFSLSTSGVGVGWIRQPPGKALEWLALIYWDDDKRYS
PSLKSRLTITKDTSKNQVVLTMTNMAPVDTATYYCAHRLSNFWSGYYTGWGQGTLVTVSSAS
TKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFLGGPSVFLFPPK
PKDTLYITREPEVTCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVFSCSVMHEAL
HNHYTQKSLSLSLGK
VL-CDR1:
(SEQ ID NO: 34)
CRSSQSLLHSNGYNYLDW
VL-CDR2:
(SEQ ID NO: 35)
LGSNRAS
VL-CDR3:
(SEQ ID NO: 36)
CMQALQTPNTF
LCVR:
(SEQ ID NO: 38)
DIVMTQSPLSLPVTPGEPASISCRSSQSLLHSNGYNYLDWYLQKPGQSPQLLIYLGSNRASG
VPDRFSGSGSGTDFTLKISRVEAEDVGVYYCMQALQTPNTFGQGTKLEIK
LC:
(SEQ ID NO: 40)
DIVMTQSPLSLPVTPGEPASISCRSSQSLLHSNGYNYLDWYLQKPGQSPQLLIYLGSNRASG
VPDRFSGSGSGTDFTLKISRVEAEDVGVYYCMQALQTPNTFGQGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGEC

### Ab-5

VH-CDR1:
(SEQ ID NO: 51)
CGVSGGSISSYYWSW
VH-CDR2:
(SEQ ID NO: 52)
GHIYDSGSTNYN
VH-CDR3:
(SEQ ID NO: 53)
CARQLWLRGAFDIW
HCVR:
(SEQ ID NO: 57)
QVQLQESGPGLVKPSETLSLTCGVSGGSISSYYWSWIRQPPGKGLEWIGHIYDSGSTNYNPS
LKSRVTISVDTSKNQFSLKLSSVTASDTAVYYCARQLWLRGAFDIWGQGTMVTVSS
HC:
(SEQ ID NO: 59)
QVQLQESGPGLVKPSETLSLTCGVSGGSISSYYWSWIRQPPGKGLEWIGHIYDSGSTNYNPS
LKSRVTISVDTSKNQFSLKLSSVTASDTAVYYCARQLWLRGAFDIWGQGTMVTVSSASTKGP
SVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
HCVR-encoding polynucleotide sequence:
(SEQ ID NO: 46)
CAAGTGCAACTTCAAGAATCTGGACCTGGACTTGTGAAACCTTCTGAAACACTTTCTCTTAC
ATGCGGAGTGTCTGGAGGATCTATCTCTTCTTATTATTGGTCTTGGATCAGACAACCTCCTG
GAAAGGGTCTAGAATGGATCGGACATATCTATGATTCTGGATCTACAAACTATAACCCTTCT
CTTAAATCTAGAGTGACAATCTCTGTGGATACATCTAAGAATCAGTTCAGTCTAAAGCTCTC
GTCAGTTACTGCGAGTGATACAGCAGTGTATTATTGCGCAAGACAACTTTGGCTTAGAGGAG
CATTTGATATCTGGGGCCAGGGAACAATGGTGACTGTCAGCAGT
VL-CDR1:
(SEQ ID NO: 54)
CTGSSGSIASNYVQW
VL-CDR2:
(SEQ ID NO: 55)
EDQQRPS
VL-CDR3:
(SEQ ID NO: 56)
CQSYDSTNQVF
LCVR:
(SEQ ID NO: 58)
NFMLTQPHSVSESPGKTITISCTGSSGSIASNYVQWYQQRPGSAPTTVIYEDQQRPSGVPDR
FSGSIDSSSNSASLTISGLKTEDEADYYCQSYDSTNQVFGGGTKLTVL
LC:
(SEQ ID NO: 60)
NFMLTQPHSVSESPGKTITISCTGSSGSIASNYVQWYQQRPGSAPTTVIYEDQQRPSGVPDR
FSGSIDSSSNSASLTISGLKTEDEADYYCQSYDSTNQVFGGGTKLTVLGQPKAAPSVTLFPP
SSEELQANKATLVCLISDFYPGAVTVAWKADSSPVKAGVETTTPSKQSNNKYAASSYLSLTP
EQWKSHRSYSCQVTHEGSTVEKTVAPTECS

### Ab-6

VH-CDR1:
(SEQ ID NO: 61)
CKVSGYTLTELSMHW
VH-CDR2:
(SEQ ID NO: 62)
GGFDPEDGETIYA
VH-CDR3:
(SEQ ID NO: 63)
CATGHQLLFYNWFDPW
HCVR:
(SEQ ID NO: 67)
QVQLVQSGAEVKKPGASVKVSCKVSGYTLTELSMHWVRQAPGKGLEWMGGFDPEDGETIYAQ
KFQGRVTMTEDTSTDTAYMELSSLRSEDTAVYYCATGHQLLFYNWFDPWGQGTLVTVSS
HC:
(SEQ ID NO: 69)
QVQLVQSGAEVKKPGASVKVSCKVSGYTLTELSMHWVRQAPGKGLEWMGGFDPEDGETIYAQ
KFQGRVTMTEDTSTDTAYMELSSLRSEDTAVYYCATGHQLLFYNWFDPWGQGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
HCVR-encoding polynucleotide sequence:
(SEQ ID NO: 47)
CAAGTGCAACTTGTGCAATCTGGAGCAGAAGTGAAGAAGCCGGGCGCATCTGTGAAAGTGTC
TTGCAAAGTGTCTGGATATACACTTACAGAACTTTCTATGCATTGGGTGAGACAAGCACCTG
GAAAGGGTCTGGAATGGATGGGAGGATTTGATCCTGAAGATGGAGAAACAATCTATGCACAG
AAGTTCCAGGGAAGAGTGACAATGACAGAAGATACATCTACAGATACAGCATATATGGAACT
TTCTTCTCTTAGATCTGAAGATACAGCAGTGTATTATTGCGCAACAGGACATCAATTACTGT
TCTATAACTGGTTTGATCCTTGGGGACAGGGGACACTTGTGACAGTGTCTTCT
VL-CDR1:
(SEQ ID NO: 64)
CTGTSSDVGGYNYVSW
VL-CDR2:
(SEQ ID NO: 65)
EVSKRPS
VL-CDR3:
(SEQ ID NO: 66)
CSSYAGSNNLVF
LCVR:
(SEQ ID NO: 68)
QSALTQPPSASGSPGQSVTISCTGTSSDVGGYNYVSWYQQHPGKAPKLMIYEVSKRPSGVPD
RFSGSKSGNTASLTVSGLQAEDEADYYCSSYAGSNNLVFGGGTKLTVL
LC:
(SEQ ID NO: 70)
QSALTQPPSASGSPGQSVTISCTGTSSDVGGYNYVSWYQQHPGKAPKLMIYEVSKRPSGVPD
RFSGSKSGNTASLTVSGLQAEDEADYYCSSYAGSNNLVFGGGTKLTVLGQPKAAPSVTLFPP
SSEELQANKATLVCLISDFYPGAVTVAWKADSSPVKAGVETTTPSKQSNNKYAASSYLSLTP
EQWKSHRSYSCQVTHEGSTVEKTVAPTECS

### Ab-7

VH-CDR1:
(SEQ ID NO: 71)
CAASGFAFSSYTMNW
VH-CDR2:
(SEQ ID NO: 72)
SSISSSSDYIFYA
VH-CDR3:
(SEQ ID NO: 73)
CARGSNTAWGGVPDAFDFW
HCVR:
(SEQ ID NO: 77)
EVQLVESGGGLVKPGGSLRLSCAASGFAFSSYTMNWVRQAPGKGLAWVSSISSSSDYIFYAD
SVKGRCTISRDNAKNSLSLQMNSLRAEDTAVYYCARGSNTAWGGVPDAFDFWGLGTWTVSS
HC:
(SEQ ID NO: 79)
EVQLVESGGGLVKPGGSLRLSCAASGFAFSSYTMNWVRQAPGKGLAWVSSISSSSDYIFYAD
SVKGRCTISRDNAKNSLSLQMNSLRAEDTAVYYCARGSNTAWGGVPDAFDFWGLGTVVTVSS
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVE
LFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
HCVR-encoding polynucleotide sequence:
(SEQ ID NO: 48)
GAAGTGCAACTTGTGGAATCTGGAGGAGGACTTGTGAAACCTGGAGGATCTCTTAGACTTTC
TTGCGCAGCATCTGGATTTGCATTCAGTAGCTATACAATGAACTGGGTGAGACAAGCACCTG
GAAAGGGCCTAGCATGGGTGTCTTCTATCTCTTCTTCTTCTGATTATATCTTCTACGCGGAT
TCTGTGAAAGGAAGATGCACAATCTCTAGAGATAACGCAAAGAATAGTCTTTCTCTTCAAAT
GAACTCTCTTAGAGCAGAAGATACAGCAGTGTATTATTGCGCAAGAGGATCTAACACAGCAT
GGGGTGGGGTCCCGGATGCATTTGATTTCTGGGGGTTGGGGACAGTGGTGACAGTGTCTTCT
VL-CDR1:
(SEQ ID NO: 74)
CTGTSSDVGRYNYVSW
VL-CDR2:
(SEQ ID NO: 75)
EVSKRPS
VL-CDR3:
(SEQ ID NO: 76)
CSSYAGSNNLVF
LCVR:
(SEQ ID NO: 78)
QSALTQPPSASGSPGQSVTISCTGTSSDVGRYNYVSWYQQHPGKAPKLMIYEVSKRPSGVPD
RFSGSKSGNTASLTVSGLQTEDEADYYCSSYAGSNNLVFGGGTKLTVL
LC:
(SEQ ID NO: 80)
QSALTQPPSASGSPGQSVTISCTGTSSDVGRYNYVSWYQQHPGKAPKLMIYEVSKRPSGVPD
RFSGSKSGNTASLTVSGLQTEDEADYYCSSYAGSNNLVFGGGTKLTVLGQPKAAPSVTLFPP
SSEELQANKATLVCLISDFYPGAVTVAWKADSSPVKAGVETTTPSKQSNNKYAASSYLSLTP
EQWKSHRSYSCQVTHEGSTVEKTVAPTECS

### Ab-8

VH-CDR1:
(SEQ ID NO: 81)
CAAPGFIVSSNYMSW
VH-CDR2:
(SEQ ID NO: 82)
SIIYSGGSTFYA
VH-CDR3:
(SEQ ID NO: 83)
CARDLQELGSLDYW
HCVR:
(SEQ ID NO: 87)
EVQLVESGGGLIQPGGSLRLSCAAPGFIVSSNYMSWVRQAPGKGLEWVSIIYSGGSTFYADS
VKGRFTISRDNSKNTLYLQMNSLRVEDTAVYYCARDLQELGSLDYWGQGTLVTVSS
HC:
(SEQ ID NO: 89)
EVQLVESGGGLIQPGGSLRLSCAAPGFIVSSNYMSWVRQAPGKGLEWVSIIYSGGSTFYADS
VKGRFTISRDNSKNTLYLQMNSLRVEDTAVYYCARDLQELGSLDYWGQGTLVTVSSASTKGP
SVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
HCVR-encoding polynucleotide sequence:
(SEQ ID NO: 49)
GAAGTGCAACTTGTGGAATCTGGAGGAGGACTTATCCAACCTGGAGGATCTCTTAGACTTTC
TTGCGCAGCACCTGGATTTATCGTGTCTTCTAACTATATGTCTTGGGTGAGACAAGCACCTG
GAAAGGGCCTAGAATGGGTGTCTATCATCTATTCTGGAGGATCTACATTCTACGCTGATTCT
GTGAAAGGAAGATTTACAATCTCTAGAGATAACTCTAAGAATACGCTTTATCTTCAAATGAA
CTCTCTTAGAGTGGAAGATACAGCAGTGTATTATTGCGCAAGAGATCTTCAAGAACTTGGAT
CTCTTGATTATTGGGGGCAGGGAACACTTGTGACAGTGTCTTCT
VL-CDR1:
(SEQ ID NO: 84)
CRASQGISSWLAW
VL-CDR2:
(SEQ ID NO: 85)
AASSLQS
VL-CDR3:
(SEQ ID NO: 86)
CQEANSFPYTF
LCVR:
(SEQ ID NO: 88)
DIQMTQSPSSVSASVGDRVTITCRASQGISSWLAWYQQKPGKAPKLLIYAASSLQSGVPSRF
SGSGSGTDFTLTISSLQPEDFATYYCQEANSFPYTFGQGTKLEIK
LC:
(SEQ ID NO: 90)
DIQMTQSPSSVSASVGDRVTITCRASQGISSWLAWYQQKPGKAPKLLIYAASSLQSGVPSRF
SGSGSGTDFTLTISSLQPEDFATYYCQEANSFPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGE

### Ab-9

VH-CDR1:
(SEQ ID NO: 91)
CAASGFIVSSNYMSW
VH-CDR2:
(SEQ ID NO: 92)
SIIYSGGSTFYA
VH-CDR3:
(SEQ ID NO: 93)
CARDLQELGSLDCW
HCVR:
(SEQ ID NO: 97)
EVQLVESGGGLIQPGGSLRLSCAASGFIVSSNYMSWVRQAPGKGLEWVSIIYSGGSTFYADS
VKGRFTISRDNSKNTLYLQMNSLRVEDTAVYYCARDLQELGSLDCWGQGTLVTVSS
HC:
(SEQ ID NO: 99)
EVQLVESGGGLIQPGGSLRLSCAASGFIVSSNYMSWVRQAPGKGLEWVSIIYSGGSTFYADS
VKGRFTISRDNSKNTLYLQMNSLRVEDTAVYYCARDLQELGSLDCWGQGTLVTVSSASTKGP
SVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
HCVR-encoding polynucleotide sequence:
(SEQ ID NO: 50)
GAAGTGCAACTTGTGGAATCTGGAGGAGGACTTATCCAACCTGGAGGATCTCTTAGACTTTC
TTGCGCAGCATCTGGATTTATCGTGTCTTCTAACTATATGTCTTGGGTGAGACAAGCACCTG
GAAAGGGCCTAGAATGGGTGTCTATCATCTATTCTGGAGGATCTACATTCTACGCTGATTCT
GTGAAAGGAAGATTTACAATCTCTAGAGATAACTCTAAGAATACGCTTTATCTTCAAATGAA
CTCTCTTAGAGTGGAAGATACAGCAGTGTATTATTGCGCAAGAGATCTTCAAGAACTTGGAT
CTCTTGATTGCTGGGGGCAGGGAACACTTGTGACAGTGTCTTCT
VL-CDR1:
(SEQ ID NO: 94)
CRASQGISSWLAW
VL-CDR2:
(SEQ ID NO: 95)
AASSLQS
VL-CDR3:
(SEQ ID NO: 96)
CQEANSFPYTF
LCVR:
(SEQ ID NO: 98)
DIQMTQSPSSVSASVGDRVTITCRASQGISSWLAWYQQKPGKAPKLLIYAASSLQSGVPSRF
SGSGSGTDFTLTISSLQPEDFATYYCQEANSFPYTFGQGTKLEIK
LC:
(SEQ ID NO: 100)
DIQMTQSPSSVSASVGDRVTITCRASQGISSWLAWYQQKPGKAPKLLIYAASSLQSGVPSRF
SGSGSGTDFTLTISSLQPEDFATYYCQEANSFPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGE

In certain embodiments, the HC and/or LC further includes a signal peptide sequence: MGWSCIILFLVATATGAHS (SEQ ID NO: 41).

### 6. Human Antibodies

The invention described herein provides human antibodies or functional fragment thereof specific for an antigen of SARS-CoV-2, such as the S1 glycoprotein.

In certain embodiments, the human antibodies are isolated/purified from convalescent patients recovering from SARS-CoV-2 infection.

In certain embodiments, the human antibodies share one or more CDR sequences with the patient-isolated antibodies described herein, such as antibodies having the same HCVR and/or LCVR CDR1-3 sequences, or antibodies having the same HCVR and/or LCVR sequences but different constant region sequences, such as modified Fc region sequence, or mutations in the constant region that enhances antibody stability and/or confers additional therapeutic benefits.

Human antibodies can be made by any suitable method. Non-limiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al, Nature 362: 255-8 (1993); onberg et al, Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Non-limiting exemplary methods also include making human antibodies using phage display libraries. See, e.g., Hoogenboom et al., J. Mol. Biol. 227: 381-8 (1992); Marks et al, J. Mol. Biol. 222: 581-97 (1991); and PCT Publication No. WO 99/10494.

Human Antibody Constant Regions

In some embodiments, a human antibody described herein comprises human constant region sequences. In some embodiments, the human heavy chain constant region is of an isotype selected from IgA, IgG, and IgD. In some embodiments, the human light chain constant region is of an isotype selected from K and X. In some embodiments, an antibody described herein comprises a human IgG constant region, for example, human IgG1, IgG2, IgG3, or IgG4. In some embodiments, an antibody or Fc fusion partner comprises a C237S mutation, for example, in an IgG1 constant region. In some embodiments, an antibody described herein comprises a human IgG2 heavy chain constant region. In some such embodiments, the IgG2 constant region comprises a P331S mutation, as described in U.S. Pat. No. 6,900,292. In some embodiments, an antibody described herein comprises a human IgG4 heavy chain constant region. In some such embodiments, an antibody described herein comprises an S241P mutation in the human IgG4 constant region. See, e.g., Angal et al. Mol. Immunol. 30(1):105-108 (1993). In some embodiments, an antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. Typically, antibodies comprising human IgG1 or IgG3 heavy chains have effector function.

In some embodiments, effector function is not desirable. For example, in some embodiments, effector function may not be desirable in treatments of inflammatory conditions and/or autoimmune disorders, such as SARS-CoV-2 induced cytokine storm. In some such embodiments, a human IgG4 or IgG2 heavy chain constant region is selected or engineered. In some embodiments, an IgG4 constant region comprises an S241P mutation.

Any of the antibodies described herein may be purified by any suitable method. Such methods include, but are not limited to, the use of affinity matrices or hydrophobic interaction chromatography. Suitable affinity ligands include the antigen and/or epitope to which the antibody binds, and ligands that bind antibody constant regions. For example, a Protein A, Protein G, Protein A/G, or an antibody affinity column may be used to bind the constant region and to purify an antibody.

In some embodiments, hydrophobic interactive chromatography (HIC), for example, a butyl or phenyl column, is also used for purifying some polypeptides. Many methods of purifying polypeptides are known in the art.

Alternatively, in some embodiments, an antibody described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al, Biotechnol. Adv. 21: 695-713 (2003).

### 7. Nucleic Acid Molecules Encoding Antibody

The invention also provides nucleic acid molecules comprising polynucleotides that encode one or more chains of an antibody described herein. In some embodiments, a nucleic acid molecule comprises a polynucleotide that encodes a heavy chain or a light chain of an antibody described herein. In some embodiments, a nucleic acid molecule comprises both a polynucleotide that encodes a heavy chain and a polynucleotide that encodes a light chain, of an antibody described herein. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody described herein comprises a nucleotide sequence that encodes a leader sequence, which, when translated, is located at the N-terminus of the heavy chain or light chain. As discussed above, the leader sequence may be the native heavy or light chain leader sequence, or may be another heterologous leader sequence.

Nucleic acid molecules may be constructed using recombinant DNA techniques conventional in the art. In some embodiments, a nucleic acid molecule is an expression vector that is suitable for expression in a selected host cell, such as a mammalian cell.

### 8. Vectors

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004). In some embodiments, a vector is chosen for in vivo expression of the subject antibody in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

### 9. Host Cells

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, heavy chains and/or light chains of the antibodies described herein may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the subject antibody. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc., Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

### EXAMPLES

### Example 1 Characterization of SARS-CoV-2 Specific Antibodies

Many antibodies specific for SARS-CoV-2 antigens were identified and further characterized herein, including antibodies Ab-1, Ab-2, Ab-3, Ab-4, Ab-5, Ab-6, Ab-7, Ab-8, and Ab-9. Among them, Ab-5 to Ab-7 are weaker binders compared to Ab-1 to Ab-4. Ab-8 and Ab-9 shares the same VL sequence with that of Ab-2, but with 1 amino acid difference in the VH region. The other Ab-2 alternatives, all with the same CDR but different framework region (FR) sequences are not listed.

The relevant sequences of these representative antibodies are listed below.

| Ab | | | |
|---|---|---|---|
| ID | VH CDR1 | VH CDR2 | VH CDR3 |
| Ab-1 | CTVSGGSISSSIYYWGW | GSIYYSGNAYYN (SEQ | CATPHTRWGPDYW (SEQ |
| | (SEQ ID NO: 1) | ID NO: 2) | ID NO: 3) |
| Ab-2 | CAASGFIVSSNYMSW (SEQ | SIIYSGGSTFYA (SEQ | CARDLQELGSLDYW (SEQ |
| | ID NO: 11) | ID NO: 12) | ID NO: 13) |
| Ab-3 | CTVSGGSISSTIYYWGW | GSIYYSGNAYYN (SEQ | CATPHTRWGPDYW (SEQ |
| | (SEQ ID NO: 21) | ID NO: 22) | ID NO: 23) |
| Ab-4 | CTFSGFSLSTSGVGVGW | ALIYWDDDKRYS (SEQ | CAHRLSNFWSGYYTGW |
| | (SEQ ID NO: 31) | ID NO: 32) | (SEQ ID NO: 33) |
| Ab-5 | CGVSGGSISSYYWSW (SEQ | GHIYDSGSTNYN (SEQ | CARQLWLRGAFDIW (SEQ |
| | ID NO: 51) | ID NO: 52) | ID NO: 53) |
| Ab-6 | CKVSGYTLTELSMHW (SEQ | GGFDPEDGETIYA (SEQ | CATGHQLLFYNWFDPW |
| | ID NO: 61) | ID NO: 62) | (SEQ ID NO: 63) |
| Ab-7 | CAASGFAFSSYTMNW (SEQ | SSISSSSDYIFYA (SEQ | CARGSNTAWGGVPDAFDFW |
| | ID NO: 71) | ID NO: 72) | (SEQ ID NO: 73) |
| Ab-8 | CAAPGFIVSSNYMSW (SEQ | SIIYSGGSTFYA (SEQ | CARDLQELGSLDYW (SEQ |
| | ID NO: 81) | ID NO: 82) | ID NO: 83) |
| Ab-9 | CAASGFIVSSNYMSW (SEQ | SIIYSGGSTFYA (SEQ | CARDLQELGSLDCW (SEQ |
| | ID NO: 91) | ID NO: 92) | ID NO: 93) |

| Ab ID | VL CDR1 | VL CDR2 | VL CDR3 |
|---|---|---|---|
| Ab-1 | CTRSSGSIASNYVLW (SEQ | EDDQRPS (SEQ ID | CQSYDGDNLVF (SEQ |
| | ID NO: 4) | NO: 5) | ID NO: 6) |
| Ab-2 | CRASQGISSWLAW (SEQ ID | AASSLQS (SEQ ID | CQEANSFPYTF (SEQ |
| | NO: 14) | NO: 15) | ID NO: 16) |
| Ab-3 | CTRSSGSIASNYVLW (SEQ | EDDQRPS (SEQ ID | CQSYDGDNLVF (SEQ |
| | ID NO: 24) | NO: 25) | ID NO: 26) |
| Ab-4 | CRSSQSLLHSNGYNYLDW | LGSNRAS (SEQ ID | CMQALQTPNTF (SEQ |
| | (SEQ ID NO: 34) | NO: 35) | ID NO: 36) |
| Ab-5 | CTGSSGSIASNYVQW (SEQ | EDQQRPS (SEQ ID | CQSYDSTNQVF (SEQ |
| | ID NO: 54) | NO: 55) | ID NO: 56) |
| Ab-6 | CTGTSSDVGGYNYVSW (SEQ | EVSKRPS (SEQ ID | CSSYAGSNNLVF (SEQ |
| | ID NO: 64) | NO: 65) | ID NO: 66) |
| Ab-7 | CTGTSSDVGRYNYVSW (SEQ | EVSKRPS (SEQ ID | CSSYAGSNNLVF (SEQ |
| | ID NO: 74) | NO: 75) | ID NO: 76) |
| Ab-8 | CRASQGISSWLAW (SEQ ID | AASSLQS (SEQ ID | CQEANSFPYTF (SEQ |
| | NO: 84) | NO: 85) | ID NO: 86) |
| Ab-9 | CRASQGISSWLAW (SEQ ID | AASSLQS (SEQ ID | CQEANSFPYTF (SEQ |
| | NO: 94) | NO: 95) | ID NO: 96) |

| | HCVR amino acid | |
|---|---|---|
| Ab ID | sequence | HCVR DNA sequence |
| Ab-1 | QLQLQESGPGLVKPSETL | CAGCTGCAGCTGCAGGAGTCGGGCCCAGGACTGGTGAAGCCTT |
| | SLTCTVSGGSISSSIYYW | CGGAGACCCTGTCCCTCACCTGCACTGTCTCTGGTGGCTCCAT |
| | GWIRQPPGKGLEWIGSIY | CAGCAGTAGTATTTACTACTGGGGCTGGATCCGCCAGCCCCCA |
| | YSGNAYYNPSLKSRVTIS | GGGAAGGGGCTGGAGTGGATTGGGAGTATCTATTATAGTGGGA |
| | VDTSKNQFSLKLNSVTAA | ACGCCTACTATAACCCGTCCCTCAAGAGTCGAGTCACCATATC |
| | DTAVYYCATPHTRWGPDY | CGTGGACACGTCCAAGAACCAGTTCTCCCTGAAGCTGAACTCT |
| | WGQGTLVTVSS (SEQ | GTGACCGCCGCAGACACGGCTGTGTATTACTGTGCGACTCCGC |
| | ID NO: 7) | ACACACGGTGGGGCCCTGACTACTGGGGCCAGGGAACCCTGGT |
| | | CACCGTCTCCTCA (SEQ ID NO: 42) |
| Ab-2 | EVQLVESGGGLIQPGGSL | GAGGTGCAGCTGGTGGAGTCTGGAGGAGGCTTGATCCAGCCTG |
| | RLSCAASGFIVSSNYMSW | GGGGGTCCCTGAGACTCTCCTGTGCAGCCTCTGGATTCATCGT |
| | VRQAPGKGLEWVSIIYSG | CAGTAGCAACTACATGAGCTGGGTCCGCCAGGCTCCAGGGAAG |
| | GSTFYADSVKGRFTISRD | GGGCTGGAGTGGGTCTCAATTATTTATAGCGGTGGTAGTACAT |
| | NSKNTLYLQMNSLRVEDT | TCTACGCAGACTCCGTGAAGGGCCGATTCACCATCTCCAGAGA |
| | AVYYCARDLQELGSLDYW | CAATTCCAAGAACACGCTGTATCTTCAAATGAACAGCCTGAGA |
| | GQGTLVTVSS (SEQ ID | GTCGAGGACACGGCCGTGTATTACTGTGCGAGAGATCTTCAGG |
| | NO: 17) | AGCTCGGCTCTCTTGACTACTGGGGCCAGGGAACCCTGGTCAC |
| | | CGTCTCCTCA (SEQ ID NO: 43) |
| Ab-3 | QLQLQESGPGLVKPSETL | CAGCTGCAGCTGCAGGAGTCGGGCCCAGGACTGGTGAAGCCTT |
| | SLTCTVSGGSISSTIYYW | CGGAGACCCTGTCCCTCACCTGCACTGTCTCTGGTGGCTCCAT |
| | GWIRQPPGKGLEWIGSIY | CAGCAGTACTATTTACTATTGGGGCTGGATCCGCCAGCCCCCA |
| | YSGNAYYNPSLKSRVTIS | GGGAAGGGGCTGGAGTGGATTGGGAGTATCTATTATAGTGGGA |
| | VDTSKNQFSLMLNSVTAS | ACGCCTACTATAACCCGTCCCTCAAGAGTCGAGTCACCATATC |
| | DTAVYYCATPHTRWGPDY | CGTGGACACGTCCAAGAACCAGTTCTCCCTGATGCTGAACTCT |
| | WGQGTLVTVSS (SEQ | GTGACCGCCTCAGACACGGCTGTGTATTACTGTGCGACTCCGC |
| | ID NO: 27) | ACACACGGTGGGGCCCTGACTACTGGGGCCAGGGAACCCTGGT |
| | | CACCGTCTCCTCA (SEQ ID NO: 44) |
| Ab-4 | QITLKESGPTLVKPTQTL | CAGATCACCTTGAAGGAGTCTGGTCCTACGCTGGTGAAACCCA |
| | TLTCTFSGFSLSTSGVGV | CACAGACCCTCACGCTGACCTGCACCTTCTCTGGGTTCTCACT |
| | GWIRQPPGKALEWLALIY | CAGCACTAGTGGAGTGGGTGTGGGCTGGATCCGTCAGCCCCCA |
| | WDDDKRYSPSLKSRLTIT | GGAAAGGCCCTGGAGTGGCTTGCACTCATTTATTGGGATGATG |
| | KDTSKNQVVLTMTNMAPV | ATAAGCGCTACAGCCCATCTCTGAAGAGCAGGCTCACCATCAC |
| | DTATYYCAHRLSNFWSGY | CAAGGACACCTCCAAAAACCAGGTGGTCCTTACAATGACCAAC |
| | YTGWGQGTLVTVSS | ATGGCCCCTGTGGACACAGCCACATATTACTGTGCACACAGAC |
| | (SEQ ID NO: 37) | TCTCCAATTTTTGGAGTGGTTATTATACTGGCTGGGGCCAGGG |
| | | AACCCTGGTCACCGTCTCCTCA (SEQ ID NO: 45) |
| Ab-5 | QVQLQESGPGLVKPSETL | CAAGTGCAACTTCAAGAATCTGGACCTGGACTTGTGAAACCTT |
| | SLTCGVSGGSISSYYWSW | CTGAAACACTTTCTCTTACATGCGGAGTGTCTGGAGGATCTAT |
| | IRQPPGKGLEWIGHIYDS | CTCTTCTTATTATTGGTCTTGGATCAGACAACCTCCTGGAAAG |
| | GSTNYNPSLKSRVTISVD | GGTCTAGAATGGATCGGACATATCTATGATTCTGGATCTACAA |
| | TSKNQFSLKLSSVTASDT | ACTATAACCCTTCTCTTAAATCTAGAGTGACAATCTCTGTGGA |
| | AVYYCARQLWLRGAFDIW | TACATCTAAGAATCAGTTCAGTCTAAAGCTCTCGTCAGTTACT |
| | GQGTMVTVSS (SEQ ID | GCGAGTGATACAGCAGTGTATTATTGCGCAAGACAACTTTGGC |
| | NO: 57) | TTAGAGGAGCATTTGATATCTGGGGCCAGGGAACAATGGTGAC |
| | | TGTCAGCAGT (SEQ ID NO: 46) |
| Ab-6 | QVQLVQSGAEVKKPGASV | CAAGTGCAACTTGTGCAATCTGGAGCAGAAGTGAAGAAGCCGG |
| | KVSCKVSGYTLTELSMHW | GCGCATCTGTGAAAGTGTCTTGCAAAGTGTCTGGATATACACT |
| | VRQAPGKGLEWMGGFDPE | TACAGAACTTTCTATGCATTGGGTGAGACAAGCACCTGGAAAG |
| | DGETIYAQKFQGRVTMTE | GGTCTGGAATGGATGGGAGGATTTGATCCTGAAGATGGAGAAA |
| | DTSTDTAYMELSSLRSED | CAATCTATGCACAGAAGTTCCAGGGAAGAGTGACAATGACAGA |
| | TAVYYCATGHQLLFYNWF | AGATACATCTACAGATACAGCATATATGGAACTTTCTTCTCTT |
| | DPWGQGTLVTVSS (SEQ | AGATCTGAAGATACAGCAGTGTATTATTGCGCAACAGGACATC |
| | ID NO: 67) | AATTACTGTTCTATAACTGGTTTGATCCTTGGGGACAGGGGAC |
| | | ACTTGTGACAGTGTCTTCT (SEQ ID NO: 47) |
| Ab-7 | EVOLVESGGGLVKPGGSL | GAAGTGCAACTTGTGGAATCTGGAGGAGGACTTGTGAAACCTG |
| | RLSCAASGFAFSSYTMNW | GAGGATCTCTTAGACTTTCTTGCGCAGCATCTGGATTTGCATT |
| | VRQAPGKGLAWVSSISSS | CAGTAGCTATACAATGAACTGGGTGAGACAAGCACCTGGAAAG |
| | SDYIFYADSVKGRCTISR | GGCCTAGCATGGGTGTCTTCTATCTCTTCTTCTTCTGATTATA |
| | DNAKNSLSLQMNSLRAED | TCTTCTACGCGGATTCTGTGAAAGGAAGATGCACAATCTCTAG |
| | TAVYYCARGSNTAWGGVP | AGATAACGCAAAGAATAGTCTTTCTCTTCAAATGAACTCTCTT |
| | DAFDFWGLGTVVTVSS | AGAGCAGAAGATACAGCAGTGTATTATTGCGCAAGAGGATCTA |
| | (SEQ ID NO: 77) | ACACAGCATGGGGTGGGGTCCCGGATGCATTTGATTTCTGGGG |
| | | GTTGGGGACAGTGGTGACAGTGTCTTCT (SEQ ID NO: |
| | | 48) |
| Ab-8 | EVQLVESGGGLIQPGGSL | GAAGTGCAACTTGTGGAATCTGGAGGAGGACTTATCCAACCTG |
| | RLSCAAPGFIVSSNYMSW | GAGGATCTCTTAGACTTTCTTGCGCAGCACCTGGATTTATCGT |
| | VRQAPGKGLEWVSIIYSG | GTCTTCTAACTATATGTCTTGGGTGAGACAAGCACCTGGAAAG |
| | GSTFYADSVKGRFTISRD | GGCCTAGAATGGGTGTCTATCATCTATTCTGGAGGATCTACAT |
| | NSKNTLYLQMNSLRVEDT | TCTACGCTGATTCTGTGAAAGGAAGATTTACAATCTCTAGAGA |
| | AVYYCARDLQELGSLDYW | TAACTCTAAGAATACGCTTTATCTTCAAATGAACTCTCTTAGA |
| | GQGTLVTVSS (SEQ ID | GTGGAAGATACAGCAGTGTATTATTGCGCAAGAGATCTTCAAG |
| | NO: 87) | AACTTGGATCTCTTGATTATTGGGGGCAGGGAACACTTGTGAC |
| | | AGTGTCTTCT (SEQ ID NO: 49) |
| Ab-9 | EVQLVESGGGLIQPGGSL | GAAGTGCAACTTGTGGAATCTGGAGGAGGACTTATCCAACCTG |
| | RLSCAASGFIVSSNYMSW | GAGGATCTCTTAGACTTTCTTGCGCAGCATCTGGATTTATCGT |
| | VRQAPGKGLEWVSIIYSG | GTCTTCTAACTATATGTCTTGGGTGAGACAAGCACCTGGAAAG |
| | GSTFYADSVKGRFTISRD | GGCCTAGAATGGGTGTCTATCATCTATTCTGGAGGATCTACAT |
| | NSKNTLYLQMNSLRVEDT | TCTACGCTGATTCTGTGAAAGGAAGATTTACAATCTCTAGAGA |
| | AVYYCARDLQELGSLDCW | TAACTCTAAGAATACGCTTTATCTTCAAATGAACTCTCTTAGA |
| | GQGTLVTVSS (SEQ ID | GTGGAAGATACAGCAGTGTATTATTGCGCAAGAGATCTTCAAG |
| | NO: 97) | AACTTGGATCTCTTGATTGCTGGGGGCAGGGAACACTTGTGAC |
| | | AGTGTCTTCT (SEQ ID NO: 50) |

| Ab ID | LCVR amino acid sequence |
|---|---|
| Ab-1 | NFMLTQPHSVSASPGKTVTVSCTRSSGSIASNYVLWYQQRPGSAPTTVIYEDDQRPSGVPDRF |
| | SASIDSSSNSASLTISGLKTEDEADYYCQSYDGDNLVFGGGTKLTVL (SEQ ID NO: 8) |
| Ab-2 | DIQMTQSPSSVSASVGDRVTITCRASQGISSWLAWYQQKPGKAPKLLIYAASSLQSGVPSRFS |
| | GSGSGTDFTLTISSLQPEDFATYYCQEANSFPYTFGQGTKLEIK (SEQ ID NO: 18) |
| Ab-3 | NFMLTQPHSVSASPGKTVTVSCTRSSGSIASNYVLWYQQRPGSAPTTVIYEDDQRPSGVPDRF |
| | SASIDSSSNSASLTISGLKTEDEADYYCQSYDGDNLVFGGGTKLTVL (SEQ ID NO: 28) |
| Ab-4 | DIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQALQTPNTFGQGTKLEIK (SEQ ID NO: |
| | 38) |
| Ab-5 | NFMLTQPHSVSESPGKTITISCTGSSGSIASNYVQWYQQRPGSAPTTVIYEDQQRPSGVPDRF |
| | SGSIDSSSNSASLTISGLKTEDEADYYCQSYDSTNQVFGGGTKLTVL (SEQ ID NO: 58) |
| Ab-6 | QSALTQPPSASGSPGQSVTISCTGTSSDVGGYNYVSWYQQHPGKAPKLMIYEVSKRPSGVPDR |
| | FSGSKSGNTASLTVSGLQAEDEADYYCSSYAGSNNLVFGGGTKLTVL (SEQ ID NO: 68) |
| Ab-7 | QSALTQPPSASGSPGQSVTISCTGTSSDVGRYNYVSWYQQHPGKAPKLMIYEVSKRPSGVPDR |
| | FSGSKSGNTASLTVSGLQTEDEADYYCSSYAGSNNLVFGGGTKLTVL (SEQ ID NO: 78) |
| Ab-8 | DIQMTQSPSSVSASVGDRVTITCRASQGISSWLAWYQQKPGKAPKLLIYAASSLQSGVPSRFS |
| | GSGSGTDFTLTISSLQPEDFATYYCQEANSFPYTFGQGTKLEIK (SEQ ID NO: 88) |
| Ab-9 | DIQMTQSPSSVSASVGDRVTITCRASQGISSWLAWYQQKPGKAPKLLIYAASSLQSGVPSRFS |
| | GSGSGTDFTLTISSLQPEDFATYYCQEANSFPYTFGQGTKLEIK (SEQ ID NO: 98) |

| Ab ID | HC a.a. sequence | LC a.a. sequence |
|---|---|---|
| Ab-1 | QLQLQESGPGLVKPSETLSLTCTVSGGSISSSIYYWGWIRQPPGK | NFMLTQPHSVSASPGKTVTVSCT |
| | GLEWIGSIYYSGNAYYNPSLKSRVTISVDTSKNQFSLKLNSVTAA | RSSGSIASNYVLWYQQRPGSAPT |
| | DTAVYYCATPHTRWGPDYWGQGTLVTVSSASTKGPSVFPLAPS | TVIYEDDQRPSGVPDRFSASIDSS |
| | SKSTSGGTAALGCLVKDYFPEPVTVSWNSGALTSGVHTFPAVLQS | SNSASLTISGLKTEDEADYYCQSY |
| | SGLYSLSSVVTVPSSSLGTQTYICNVNHKPSNTKVDKKVEPKSCDK | DGDNLVFGGGTKLTVLGQPKAA |
| | THTCPPCPAPELLGGPSVFLFPPKPKDTLMISRTPEVTCVVVDVSH | PSVTLFPPSSEELQANKATLVCLIS |
| | EDPEVKFNWYVDGVEVHNAKTKPREEQYNSTYRVVSVLTVLHQD | DFYPGAVTVAWKADSSPVKAGVE |
| | WLNGKEYKCKVSNKALPAPIEKTISKAKGQPREPQVYTLPPSREE | TTTPSKQSNNKYAASSYLSLTPEQ |
| | MTKNQVSLTCLVKGFYPSDIAVEWESNGQPENNYKTTPPVLDSD | WKSHRSYSCQVTHEGSTVEKTVA |
| | GSFFLYSKLTVDKSRWQQGNVFSCSVMHEALHNHYTQKSLSLSP | PTECS (SEQ ID NO: 10) |
| | GK (SEQ ID NO: 9) | |
| Ab-2 | EVQLVESGGGLIQPGGSLRLSCAASGFIVSSNYMSWVRQAPGK | DIQMTQSPSSVSASVGDRVTITC |
| | GLEWVSIIYSGGSTFYADSVKGRFTISRDNSKNTLYLQMNSLRVE | RASQGISSWLAWYQQKPGKAPK |
| | DTAVYYCARDLQELGSLDYWGQGTLVTVSSASTKGPSVFPLAPS | LLIYAASSLQSGVPSRFSGSGSGT |
| | SKSTSGGTAALGCLVKDYFPEPVTVSWNSGALTSGVHTFPAVLQS | DFTLTISSLQPEDFATYYCQEANS |
| | SGLYSLSSVVTVPSSSLGTQTYICNVNHKPSNTKVDKKVEPKSCDK | FPYTFGQGTKLEIKRTVAAPSVFIF |
| | THTCPPCPAPELLGGPSVFLFPPKPKDTLMISRTPEVTCVVVDVSH | PPSDEQLKSGTASVVCLLNNFYPR |
| | EDPEVKFNWYVDGVEVHNAKTKPREEQYNSTYRVVSVLTVLHQD | EAKVQWKVDNALQSGNSQESVT |
| | WLNGKEYKCKVSNKALPAPIEKTISKAKGQPREPQVYTLPPSREE | EQDSKDSTYSLSSTLTLSKADYEKH |
| | MTKNQVSLTCLVKGFYPSDIAVEWESNGQPENNYKTTPPVLDSD | KVYACEVTHQGLSSPVTKSFNRGE |
| | GSFFLYSKLTVDKSRWQQGNVFSCSVMHEALHNHYTQKSLSLSP | C (SEQ ID NO: 20) |
| | GK (SEQ ID NO: 19) | |
| Ab-3 | QLQLQESGPGLVKPSETLSLTCTVSGGSISSTIYYWGWIRQPPGK | NFMLTQPHSVSASPGKTVTVSCT |
| | GLEWIGSIYYSGNAYYNPSLKSRVTISVDTSKNQFSLMLNSVTAS | RSSGSIASNYVLWYQQRPGSAPT |
| | DTAVYYCATPHTRWGPDYWGQGTLVTVSSASTKGPSVFPLAPS | TVIYEDDQRPSGVPDRFSASIDSS |
| | SKSTSGGTAALGCLVKDYFPEPVTVSWNSGALTSGVHTFPAVLQS | SNSASLTISGLKTEDEADYYCQSY |
| | SGLYSLSSVVTVPSSSLGTQTYICNVNHKPSNTKVDKKVEPKSCDK | DGDNLVFGGGTKLTVLGQPKAA |
| | THTCPPCPAPELLGGPSVFLFPPKPKDTLMISRTPEVTCVVVDVSH | PSVTLFPPSSEELQANKATLVCLIS |
| | EDPEVKFNWYVDGVEVHNAKTKPREEQYNSTYRVVSVLTVLHQD | DFYPGAVTVAWKADSSPVKAGVE |
| | WLNGKEYKCKVSNKALPAPIEKTISKAKGQPREPQVYTLPPSREE | TTTPSKQSNNKYAASSYLSLTPEQ |
| | MTKNQVSLTCLVKGFYPSDIAVEWESNGQPENNYKTTPPVLDSD | WKSHRSYSCQVTHEGSTVEKTVA |
| | GSFFLYSKLTVDKSRWQQGNVFSCSVMHEALHNHYTQKSLSLSP | PTECS (SEQ ID NO: 30) |
| | GK (SEQ ID NO: 29) | |
| Ab-4 | QITLKESGPTLVKPTQTLTLTCTFSGFSLSTSGVGVGWIRQPPGK | DIVMTQSPLSLPVTPGEPASISCR |
| | ALEWLALIYWDDDKRYSPSLKSRLTITKDTSKNQVVLTMTNMA | SSQSLLHSNGYNYLDWYLQKPG |
| | PVDTATYYCAHRLSNFWSGYYTGWGQGTLVTVSSASTKGPSVF | QSPQLLIYLGSNRASGVPDRFSGS |
| | PLAPSSKSTSGGTAALGCLVKDYFPEPVTVSWNSGALTSGVHTFP | GSGTDFTLKISRVEAEDVGVYYC |
| | AVLQSSGLYSLSSVVTVPSSSLGTQTYICNVNHKPSNTKVDKKVEP | MQALQTPNTFGQGTKLEIKRTVA |
| | KSCDKTHTCPPCPAPELLGGPSVFLFPPKPKDTLMISRTPEVTCVV | APSVFIFPPSDEQLKSGTASVVCLL |
| | VDVSHEDPEVKFNWYVDGVEVHNAKTKPREEQYNSTYRVVSVLT | NNFYPREAKVQWKVDNALQSGN |
| | VLHQDWLNGKEYKCKVSNKALPAPIEKTISKAKGQPREPQVYTLP | SQESVTEQDSKDSTYSLSSTLTLSK |
| | PSREEMTKNQVSLTCLVKGFYPSDIAVEWESNGQPENNYKTTPPV | ADYEKHKVYACEVTHQGLSSPVTK |
| | LDSDGSFFLYSKLTVDKSRWQQGNVFSCSVMHEALHNHYTQKSL | SFNRGEC (SEQ ID NO: |
| | SLSPGK (SEQ ID NO: 39) | 40) |
| Ab-5 | QVQLQESGPGLVKPSETLSLTCGVSGGSISSYYWSWIRQPPGKG | NFMLTQPHSVSESPGKTITISCTG |
| | LEWIGHIYDSGSTNYNPSLKSRVTISVDTSKNQFSLKLSSVTASDT | SSGSIASNYVQWYQQRPGSAPT |
| | AVYYCARQLWLRGAFDIWGQGTMVTVSSASTKGPSVFPLAPSS | TVIYEDQQRPSGVPDRFSGSIDSS |
| | KSTSGGTAALGCLVKDYFPEPVTVSWNSGALTSGVHTFPAVLQSS | SNSASLTISGLKTEDEADYYCQSY |
| | GLYSLSSVVTVPSSSLGTQTYICNVNHKPSNTKVDKKVEPKSCDKT | DSTNQVFGGGTKLTVLGQPKAAP |
| | HTCPPCPAPELLGGPSVFLFPPKPKDTLMISRTPEVTCVVVDVSHE | SVTLFPPSSEELQANKATLVCLISD |
| | DPEVKFNWYVDGVEVHNAKTKPREEQYNSTYRVVSVLTVLHQD | FYPGAVTVAWKADSSPVKAGVET |
| | WLNGKEYKCKVSNKALPAPIEKTISKAKGQPREPQVYTLPPSREE | TTPSKQSNNKYAASSYLSLTPEQW |
| | MTKNQVSLTCLVKGFYPSDIAVEWESNGQPENNYKTTPPVLDSD | KSHRSYSCQVTH EGSTVEKTVAPT |
| | GSFFLYSKLTVDKSRWQQGNVFSCSVMHEALHNHYTQKSLSLSP | ECS (SEQ ID NO: 60) |
| | GK (SEQ ID NO: 59) | |
| Ab-6 | QVQLVQSGAEVKKPGASVKVSCKVSGYTLTELSMHWVRQAPG | QSALTQPPSASGSPGQSVTISCTG |
| | KGLEWMGGFDPEDGETIYAQKFQGRVTMTEDTSTDTAYMELS | TSSDVGGYNYVSWYQQHPGKA |
| | SLRSEDTAVYYCATGHQLLFYNWFDPWGQGTLVTVSSASTKGP | PKLMIYEVSKRPSGVPDRFSGSKS |
| | SVFPLAPSSKSTSGGTAALGCLVKDYFPEPVTVSWNSGALTSGVH | GNTASLTVSGLQAEDEADYYCSS |
| | TFPAVLQSSGLYSLSSVVTVPSSSLGTQTYICNVNHKPSNTKVDKK | YAGSNNLVFGGGTKLTVLGQPKA |
| | VEPKSCDKTHTCPPCPAPELLGGPSVFLFPPKPKDTLMISRTPEVTC | APSVTLFPPSSEELQANKATLVCLI |
| | VVVDVSHEDPEVKFNWYVDGVEVHNAKTKPREEQYNSTYRVVS | SDFYPGAVTVAWKADSSPVKAGV |
| | VLTVLHQDWLNGKEYKCKVSNKALPAPIEKTISKAKGQPREPQVY | ETTTPSKQSNNKYAASSYLSLTPEQ |
| | TLPPSREEMTKNQVSLTCLVKGFYPSDIAVEWESNGQPENNYKTT | WKSHRSYSCQVTHEGSTVEKTVA |
| | PPVLDSDGSFFLYSKLTVDKSRWQQGNVFSCSVMHEALHNHYTQ | PTECS (SEQ ID NO: 70) |
| | KSLSLSPGK (SEQ ID NO: 69) | |
| Ab-7 | EVQLVESGGGLVKPGGSLRLSCAASGFAFSSYTMNWVRQAPGK | QSALTQPPSASGSPGQSVTISCTG |
| | GLAWVSSISSSSDYIFYADSVKGRCTISRDNAKNSLSLQMNSLRA | TSSDVGRYNYVSWYQQHPGKAP |
| | EDTAVYYCARGSNTAWGGVPDAFDFWGLGTVVTVSSASTKGP | KLMIYEVSKRPSGVPDRFSGSKS |
| | SVFPLAPSSKSTSGGTAALGCLVKDYFPEPVTVSWNSGALTSGVH | GNTASLTVSGLQTEDEADYYCSS |
| | TFPAVLQSSGLYSLSSVVTVPSSSLGTQTYICNVNHKPSNTKVDKK | YAGSNNLVFGGGTKLTVLGQPKA |
| | VEPKSCDKTHTCPPCPAPELLGGPSVFLFPPKPKDTLMISRTPEVTC | APSVTLFPPSSEELQANKATLVCLI |
| | VVVDVSHEDPEVKFNWYVDGVEVHNAKTKPREEQYNSTYRVVS | SDFYPGAVTVAWKADSSPVKAGV |
| | VLTVLHQDWLNGKEYKCKVSNKALPAPIEKTISKAKGQPREPQVY | ETTTPSKQSNNKYAASSYLSLTPEQ |
| | TLPPSREEMTKNQVSLTCLVKGFYPSDIAVEWESNGQPENNYKTT | WKSHRSYSCQVTHEGSTVEKTVA |
| | PPVLDSDGSFFLYSKLTVDKSRWQQGNVFSCSVMHEALHNHYTQ | PTECS (SEQ ID NO: 80) |
| | KSLSLSPGK (SEQ ID NO: 7 9) | |
| Ab-8 | EVQLVESGGGLIQPGGSLRLSCAAPGFIVSSNYMSWVRQAPGK | DIQMTQSPSSVSASVGDRVTITC |
| | GLEWVSHYSGGSTFYADSVKGRFTISRDNSKNTLYLQMNSLRVE | RASQGISSWLAWYQQKPGKAPK |
| | DTAVYYCARDLQELGSLDYWGQGTLVTVSSASTKGPSVFPLAPS | LLIYAASSLQSGVPSRFSGSGSGT |
| | SKSTSGGTAALGCLVKDYFPEPVTVSWNSGALTSGVHTFPAVLQS | DFTLTISSLQPEDFATYYCQEANS |
| | SGLYSLSSVVTVPSSSLGTQTYICNVNHKPSNTKVDKKVEPKSCDK | FPYTFGQGTKLEIKRTVAAPSVFIF |
| | THTCPPCPAPELLGGPSVFLFPPKPKDTLMISRTPEVTCVVVDVSH | PPSDEQLKSGTASVVCLLNNFYPR |
| | EDPEVKFNWYVDGVEVHNAKTKPREEQYNSTYRVVSVLTVLHQD | EAKVQWKVDNALQSGNSQESVT |
| | WLNGKEYKCKVSNKALPAPIEKTISKAKGQPREPQVYTLPPSREE | EQDSKDSTYSLSSTLTLSKADYEKH |
| | MTKNQVSLTCLVKGFYPSDIAVEWESNGQPENNYKTTPPVLDSD | KVYACEVTHQGLSSPVTKSFNRGE |
| | GSFFLYSKLTVDKSRWQQGNVFSCSVMHEALHNHYTQKSLSLSP | (SEQ ID NO: 90) |
| | GK (SEQ ID NO: 89) | |
| Ab-9 | EVQLVESGGGLIQPGGSLRLSCAASGFIVSSNYMSWVRQAPGK | DIQMTQSPSSVSASVGDRVTITC |
| | GLEWVSHYSGGSTFYADSVKGRFTISRDNSKNTLYLQMNSLRVE | RASQGISSWLAWYQQKPGKAPK |
| | DTAVYYCARDLQELGSLDCWGQGTLVTVSSASTKGPSVFPLAPS | LLIYAASSLQSGVPSRFSGSGSGT |
| | SKSTSGGTAALGCLVKDYFPEPVTVSWNSGALTSGVHTFPAVLQS | DFTLTISSLQPEDFATYYCQEANS |
| | SGLYSLSSVVTVPSSSLGTQTYICNVNHKPSNTKVDKKVEPKSCDK | FPYTFGQGTKLEIKRTVAAPSVFIF |
| | THTCPPCPAPELLGGPSVFLFPPKPKDTLMISRTPEVTCVVVDVSH | PPSDEQLKSGTASVVCLLNNFYPR |
| | EDPEVKFNWYVDGVEVHNAKTKPREEQYNSTYRVVSVLTVLHQD | EAKVQWKVDNALQSGNSQESVT |
| | WLNGKEYKCKVSNKALPAPIEKTISKAKGQPREPQVYTLPPSREE | EQDSKDSTYSLSSTLTLSKADYEKH |
| | MTKNQVSLTCLVKGFYPSDIAVEWESNGQPENNYKTTPPVLDSD | KVYACEVTHQGLSSPVTKSFNRGE |
| | GSFFLYSKLTVDKSRWQQGNVFSCSVMHEALHNHYTQKSLSLSP | (SEQ ID NO: 100) |
| | GK (SEQ ID NO: 99) | |

Signal peptide sequences are not included; the variable regions are in bold font; and the constant regions are in normal font

HC and LC signal peptide sequence: MGWSCILFLVATATGAHS (SEQ ID NO: 41)

IgG4 versions of Ab-1 to Ab-4 have also been generated. These antibodies have the same light chain sequences, and heavy chain variable regions (bold sequences) as the sequences above, but have distinct IgG4 heavy chain region sequences, as shown below. The corresponding LC and HC leader sequences are also provided. The only exception is IgG4 Ab-4, in which its 33^{rd} light chain variable region residue N is substituted by Q.

| | | HC Leader | LC Leader |
|---|---|---|---|
| Ab ID | IgG4 HC a.a. sequence (without leader) | sequence | sequence |
| Ab-1 | QLQLQESGPGLVKPSETLSLTCTVSGGSISSSIYYWGWIRQ | MKHLWFFLL | MAWAPLLLT |
| | PPGKGLEWIGSIYYSGNAYYNPSLKSRVTISVDTSKNQFSL | LVAAPRWVL | LLAHCTGSW |
| | KLNSVTAADTAVYYCATPHTRWGPDYWGQGTLVTVSSASTK | S (SEQ ID | A (SEQ ID |
| | GPSVFPLAPCSRSTSESTAALGCLVKDYFPEPVTVSWNSGA | NO: 105) | NO: 109) |
| | LTSGVHTFPAVLQSSGLYSLSSVVTVPSSSLGTKTYTCNVD | | |
| | HKPSNTKVDKRVESKYGPPCPPCPAPEFLGGPSVFLFPPKP | | |
| | KDTLYITREPEVTCVVVDVSQEDPEVQFNWYVDGVEVHNAK | | |
| | TKPREEQFNSTYRVVSVLTVLHQDWLNGKEYKCKVSNKGLP | | |
| | SSIEKTISKAKGQPREPQVYTLPPSQEEMTKNQVSLTCLVK | | |
| | GFYPSDIAVEWESNGQPENNYKTTPPVLDSDGSFFLYSRLT | | |
| | VDKSRWQEGNVFSCSVMHEALHNHYTQKSLSLSLGK (SEQ | | |
| | ID NO: 101) | | |
| Ab-2 | EVQLVESGGGLIQPGGSLRLSCAASGFIVSSNYMSWVRQAP | MEFGLSWLF | MDMRVPAQL |
| | GKGLEWVSIIYSGGSTFYADSVKGRFTISRDNSKNTLYLQM | LVAILKGVQ | LGLLLLWLR |
| | NSLRVEDTAVYYCARDLQELGSLDYWGQGTLVTVSSASTKG | C (SEQ ID | GARC (SEQ |
| | PSVFPLAPCSRSTSESTAALGCLVKDYFPEPVTVSWNSGAL | NO: 106) | ID NO: |
| | TSGVHTFPAVLQSSGLYSLSSVVTVPSSSLGTKTYTCNVDH | | 110) |
| | KPSNTKVDKRVESKYGPPCPPCPAPEFLGGPSVFLFPPKPK | | |
| | DTLYITREPEVTCVVVDVSQEDPEVQFNWYVDGVEVHNAKT | | |
| | KPREEQFNSTYRVVSVLTVLHQDWLNGKEYKCKVSNKGLPS | | |
| | SIEKTISKAKGQPREPQVYTLPPSQEEMTKNQVSLTCLVKG | | |
| | FYPSDIAVEWESNGQPENNYKTTPPVLDSDGSFFLYSRLTV | | |
| | DKSRWQEGNVFSCSVMHEALHNHYTQKSLSLSLGK (SEQ | | |
| | ID NO: 102) | | |
| Ab-3 | QLQLQESGPGLVKPSETLSLTCTVSGGSISSTIYYWGWIRQ | MKHLWFFLL | MAWAPLLLT |
| | PPGKGLEWIGSIYYSGNAYYNPSLKSRVTISVDTSKNQFSL | LVAAPRWVL | LLAHCTGSW |
| | MLNSVTASDTAVYYCATPHTRWGPDYWGQGTLVTVSSASTK | S (SEQ ID | A (SEQ ID |
| | GPSVFPLAPCSRSTSESTAALGCLVKDYFPEPVTVSWNSGA | NO: 107) | NO: 111) |
| | LTSGVHTFPAVLQSSGLYSLSSVVTVPSSSLGTKTYTCNVD | | |
| | HKPSNTKVDKRVESKYGPPCPPCPAPEFLGGPSVFLFPPKP | | |
| | KDTLYITREPEVTCVVVDVSQEDPEVQFNWYVDGVEVHNAK | | |
| | TKPREEQFNSTYRVVSVLTVLHQDWLNGKEYKCKVSNKGLP | | |
| | SSIEKTISKAKGQPREPQVYTLPPSQEEMTKNQVSLTCLVK | | |
| | GFYPSDIAVEWESNGQPENNYKTTPPVLDSDGSFFLYSRLT | | |
| | VDKSRWQEGNVFSCSVMHEALHNHYTQKSLSLSLGK (SEQ | | |
| | ID NO: 103) | | |
| Ab-4 | QITLKESGPTLVKPTQTLTLTCTFSGFSLSTSGVGVGWIRQ | MDTLCSTLL | MDMRVPAQL |
| | PPGKALEWLALIYWDDDKRYSPSLKSRLTITKDTSKNQVVL | LLTIPSWVL | LGLLLLWLR |
| | TMTNMAPVDTATYYCAHRLSNFWSGYYTGWGQGTLVTVSSA | S (SEQ ID | GARC (SEQ |
| | STKGPSVFPLAPCSRSTSESTAALGCLVKDYFPEPVTVSWN | NO: 108) | ID NO: |
| | SGALTSGVHTFPAVLQSSGLYSLSSVVTVPSSSLGTKTYTC | | 112) |
| | NVDHKPSNTKVDKRVESKYGPPCPPCPAPEFLGGPSVFLFP | | |
| | PKPKDTLYITREPEVTCVVVDVSQEDPEVQFNWYVDGVEVH | | |
| | NAKTKPREEQFNSTYRVVSVLTVLHQDWLNGKEYKCKVSNK | | |
| | GLPSSIEKTISKAKGQPREPQVYTLPPSQEEMTKNQVSLTC | | |
| | LVKGFYPSDIAVEWESNGQPENNYKTTPPVLDSDGSFFLYS | | |
| | RLTVDKSRWQEGNVFSCSVMHEALHNHYTQKSLSLSLGK | | |
| | (SEQ ID NO: 104) | | |

Ab-4 IgG4 LC a.a. sequence (without leader sequence):

(SEQ ID NO: 113)
DIVMTQSPLSLPVTPGEPASISCRSSQSLLHSQGYNYLDWYLQKPGQSP
QLLIYLGSNRASGVPDRFSGSGSGTDFTLKISRVEAEDVGVYYCMQALQ
TPNTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

Ab-4 IgG4 LCVR a.a. sequence:

(SEQ ID NO: 114)
DIVMTQSPLSLPVTPGEPASISCRSSQSLLHSQGYNYLDWYLQKPGQSP
QLLIYLGSNRASGVPDRFSGSGSGTDFTLKISRVEAEDVGVYYCMQALQ
TPNTFGQGTKLEIK

| Ab ID | VL CDR1 | VL CDR2 | VL CDR3 |
|---|---|---|---|
| Ab-4 | CRSSQSLLHSQGYNYLDW | LGSNRAS (SEQ ID | CMQALQTPNTF (SEQ |
| (IgG4) | (SEQ ID NO: 115) | NO: 35) | ID NO: 36) |

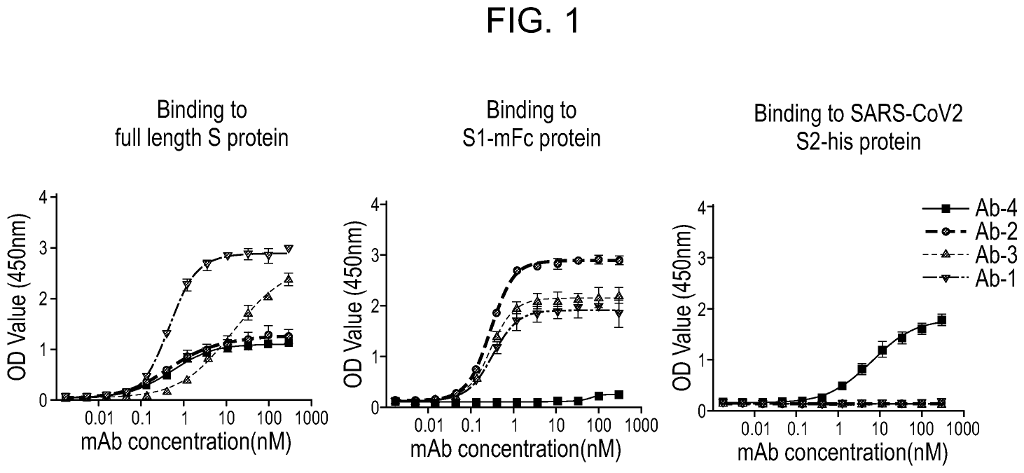

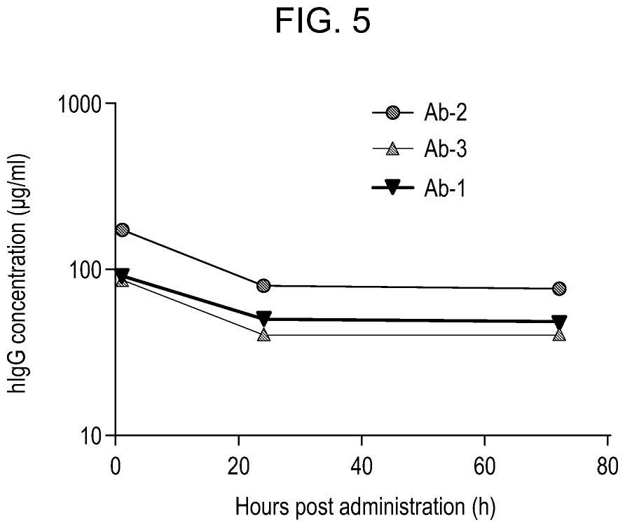

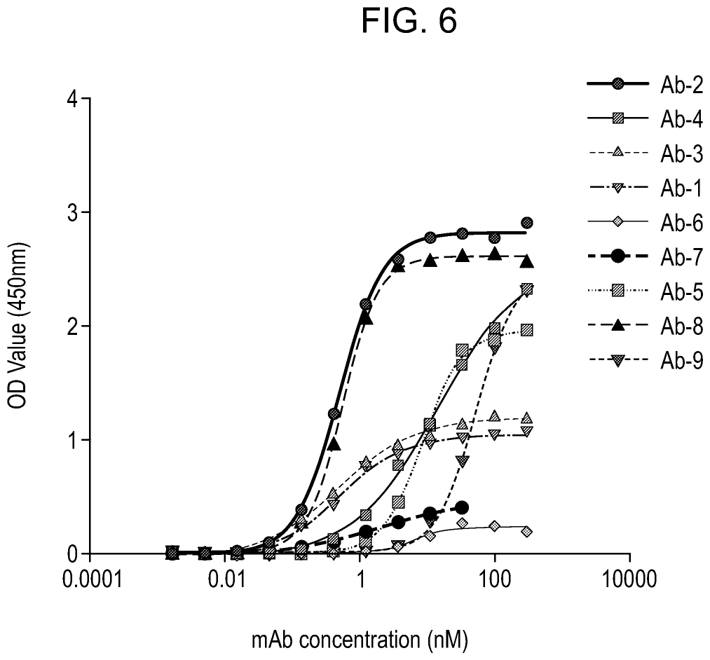

The antibodies of the invention have been demonstrated to be able to bind to the spike protein of the SARS-CoV-2 virus, based on binding assays for S1 and S2 domains, as well as the full length of the S protein. ELISA results showed that at least 20 antibodies bound to the viral S protein with different affinity, among which Ab-2 was the strongest binder, and Ab-4 is another top candidate which also binds to S full length protein (see FIG. 6). Ab-1 and Ab-3 are two other binding candidates with reduced Bₘₐₓ and EC50 values comparing with Ab-2 and Ab-4, however, with nM level binding affinity, nevertheless. These two antibodies share the same heavy chain, with difference in their light chains. ELISA results showed that the antibodies can effectively bind to full length S protein of SARS-CoV-2 with EC50 ranging from 10⁻⁹-10⁻¹⁰ M (FIG. 5, left panel). Interestingly, when the binding of the specific domain of S protein was evaluated, Ab-1 to Ab-3 were shown to be binders to the S1 fragment, while Ab-4 to be a binder to the S2 fragment (middle and right panels of FIG. 1), respectively, showing the diversity of mAb being identified.

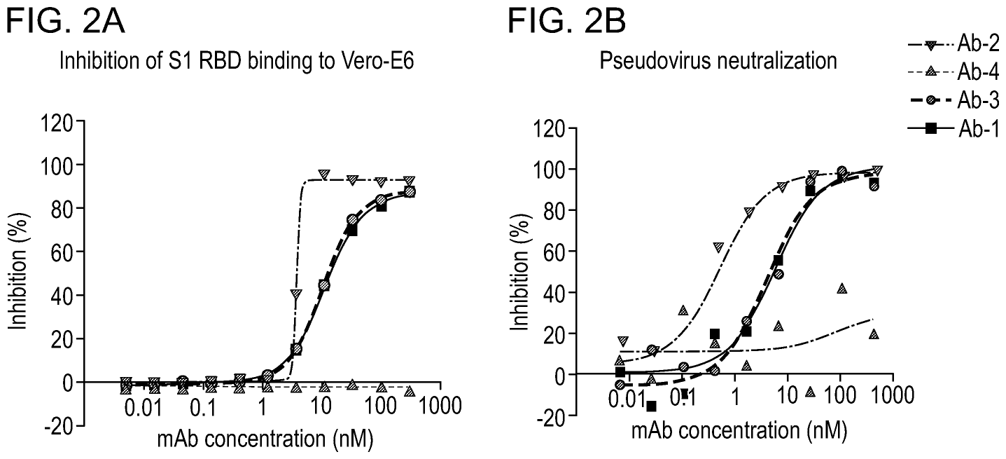

Next, the blocking effects of the top four antibodies on the binding of S1 to Vero E6 cell line was analyzed by FACS analysis (FIG. 2A). As expected, Ab-1 to Ab-3 blocked S1 binding to E6 at the nM range, while S2 binder Ab-4 did not affect S1 binding to the receptor on Vero E6 cells as expected.

The blocking activities of these antibodies were also tested for blocking S1 binding to ACE2 using ELISA assay (data not shown). Again, Ab-1, Ab-2, & Ab-3 showed potent blocking activities with potency at 10⁻⁹-10⁻¹⁰ M, and Ab-4 did not show any blocking activity on S1 binding to ACE2.

Next, the neutralizing activities of these antibodies against HIV-vectored pseudovirus were examined. Antibodies Ab-2 exhibited potent neutralizing activity, with IC50 of 0.53 nM on average. Ab-3 and Ab-1 showed IC50s at 5.80 nM and 4.07 nM respectively, whereas Ab-4 displayed no neutralizing activities (FIG. 2B). These antibody activities from S protein binding to blockage of S1 binding to Vero E6 cells and further to pesudovirus neutralization suggested that S1 binding is a critical step for virus to bind to host cells, and blocking of S1 binding to host cells could potentially be translated into anti-viral activities. The results are summarized in the table below.

| EC50/EC90 or IC50/IC90 (nM) | | Ab-2 | Ab-4 | Ab-3 | Ab-1 |
|---|---|---|---|---|---|
| Binding to S protein | EC50 | 0.50 | 14.23 | 0.50 | 0.53 |
| | EC90 | 2.56 | 327.6 | 9.36 | 6.03 |
| Inhibition of S1-Fc binding to | IC50 | 3.78 | >300 | 10.60 | 11.13 |
| Cero-E6 cells | IC90 | 4.53 | >300 | 46.99 | 58.18 |
| Pseudo-virus neutralization | IC50 | 0.53 | >300 | 5.80 | 4.07 |
| | IC90 | 4.76 | >300 | 52.19 | 36.64 |

Sequence analysis showed that each cluster of antibodies shared at least 93% sequence identity. The difference within the same sequence cluster may result from somatic hypermutation in which point mutations accumulated in the antibody. Because the somatic hypermutation does not distinguish between favorable and unfavorable mutations, 12 closely similar sequences in the cluster comprising Ab-2 were chosen to compare their activity experimentally.

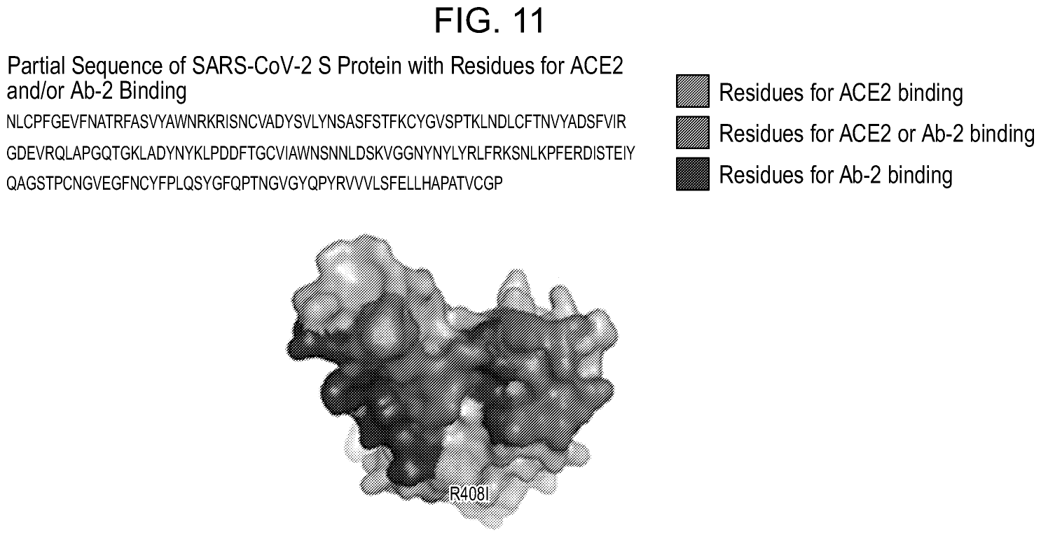

As shown in FIGS. 8A and 8B, 11 antibodies (labeled as “2A2A-x” wherein x is 1-4, 9-11, 14, 15, 18 and 19) from the same family of Ab-2 (also labeled as “2A2A”) demonstrated various levels of binding abilities to the full-length S protein as well as the abilities to block the S1 protein binding to human ACE2 receptor. However, Ab-2 demonstrated the best potency in both assays, with a few close family members showing similar activities while 2A2A-4 with 1 amino acid difference in CDR1, and 2A2A-19 with 1 amino acid difference in CDR3, others with differences in the FR regions.

| | 2A2A | 2A2A-4 | 2A2A-3 | 2A2A-1 | 2A2A-2 | 2A2A-10 | 2A2A-9 | 2A2A-11 | 2A2A-15 | 2A2A-18 | 2A2A-19 | 2A2A-14 |
|---|---|---|---|---|---|---|---|---|---|---|---|---|
| EC50 | 0.55 | 0.57 | 0.66 | 0.68 | 0.81 | 1.05 | 1.27 | 4.35 | 4.65 | 14.12 | 56.5 | ~124.2 |
| IC50 (nM) | [symbol] | [symbol] | 1.62 | [symbol] | 2.31 | [symbol] | [symbol] | 4.14 | [symbol] | [symbol] | [symbol] | No inh |
| Difference | | 1AA | [symbol] | [symbol] | [symbol] | 2AA | 1AA | [symbol] | 1AA | 1AA | 1AA | 1AA |
| | | CDR1 | FR4 | FR4 | FR4 | FR4 | FR4 | FR1 | FR3 | FR1 | CDR3 | FR3 |

[symbol]

indicates data missing or illegible when filed

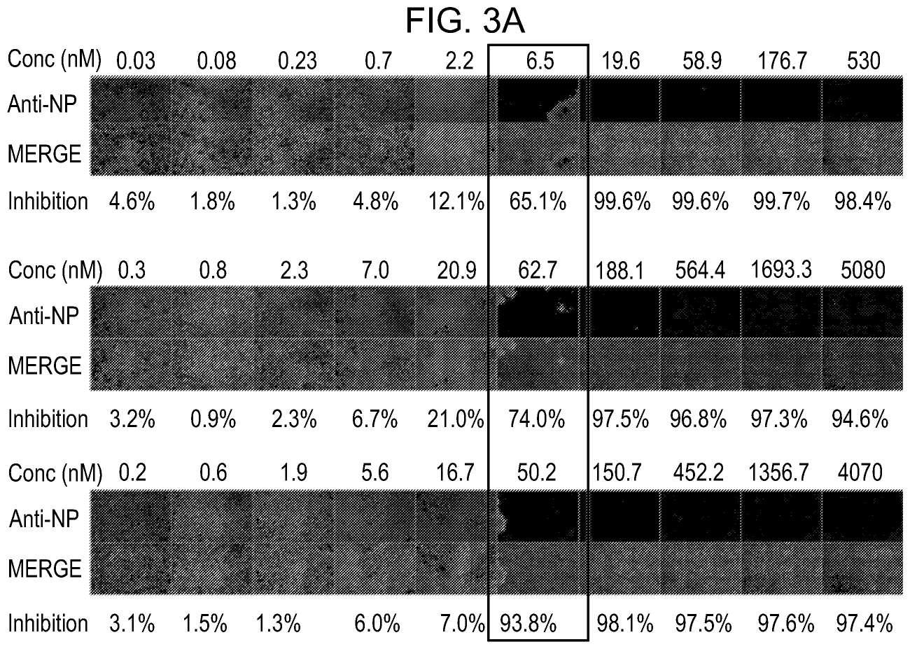

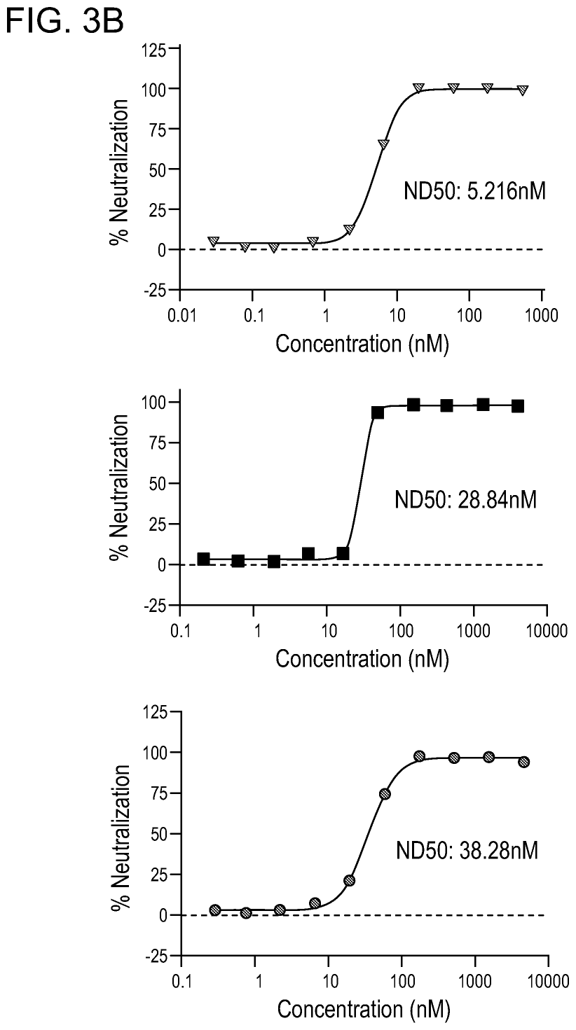

The neutralizing activities of the lead antibodies Ab-1 to Ab-3 were further confirmed in live SARS-CoV-2 virus entry assay. Vero E6 cells were infected with SARS-CoV-2 virus at 100 TCID50 in the presence of the lead antibodies at different concentrations. Using fluorescent labeled nuclear protein of SAR-CoV-2, infected cells can be observed by florescence microscopy. Antibody Ab-2 demonstrated strong anti-virus activities—more than 50% inhibition at 6.5 nM, while Ab-3 demonstrated 74% inhibition at 62.7 nM, and Ab-1 showed 93.8% inhibition at 50.2 nM (FIG. 3A). The ND50s and ND90s have been further calculated through dose responding curve. Ab-2 has a ND50 of 0.751 nM, a ND90 of 1.682 nM (FIG. 3B, left panel); Ab-1 has a ND50 of 4.153 nM, a ND90 of 6.1 nM (FIG. 3B, middle panel); and Ab-3 has a ND50 of 5.512 nM, a ND90 of 24.08 nM (FIG. 3B, right panel).

| | Ab-1 | Ab-2 | Ab-3 |
|---|---|---|---|
| ND50 (μg/mL) | 4.153 | 0.751 | 5.512 |
| ND90 (μg/mL) | 6.100 | 1.682 | 14.076 |

As the pandemics progressed, the mutations located in S protein accumulated and may confer selective advantages in transmission and resistance to the antibody intervention. Thus, the affinity of Ab-2 for 4 dominant spike RBD mutations, and its ability to competing with ACE2 for RBD binding were examined. SPR results showed that Ab-2 bound to mutations with similar affinity as to the wild-type S protein. Ab-2 also blocked the interaction of ACE2 with all RBD mutations (data not shown).

### Example 2 Pharmacological Profile of the Neutralizing Antibodies

The three most potent antibodies binding to S1 protein has been further profiled pharmacologically. A snapshot PK study at 1 h, 24 h and 72 h separately in wild-type (WT) mice showed good PK profile. While no significant difference was observed among the 3 Abs, Ab-2 showed slightly better exposure comparing with Ab-1 and Ab-3 (FIG. 5). This results provide further confidence that these antibodies can be utilized for further development as a drug candidate(s) to treat COVID-19 patients.

### Example 3 IgG4 Monoclonal Antibodies have Comparable Activity

This example demonstrates that certain subject antibodies with hIgG4 constant region (which does not engage or minimally engages FcγR), compared to their counterpart with the hIgG1 constant region, have comparable binding affinity for the SARS-CoV-2 S1 antigen, pseudovirus neutralization activity, and live virus neutralization activity. Such hIgG4 antibodies also have comparable favorable developability characteristics.

For binding affinity assay, SPR (surface plasmon resonance) with anti-hIgG Fc immobilized on CM5 chip was used to capture several subject monoclonal antibodies with hIgG1 vs. hIgG4 constant region, respectively, in order to compare their respective binding affinity for the soluble S1 RBD domain. The results are summarized in the table below.

| | | | | |
|---|---|---|---|---|
| Antibody | Kₐ (1/Ms) | k_{d} (1/s) | K_{D} (M) | Note |

| | | |
|---|---|---|
| Ab-4 (IgG1) | No binding | NA |

| | | | | |
|---|---|---|---|---|
| Ab-1 (IgG1) | 1.32E+06 | 1.69E−04 | 1.29E−10 | 1:1 binding |
| Ab-2 (IgG4) | 2.54E+05 | 7.62E−05 | 3.00E−10 | 1:1 binding |
| Ab-3 (IgG4) | 1.92E+06 | 2.83E−04 | 1.47E−10 | 1:1 binding |

As indicated before, Ab-4 binds S2 but not S1 protein, which served as a negative control. The measured K_{D} values for the different antibodies in IgG1 vs. IgG4 formats were comparable.

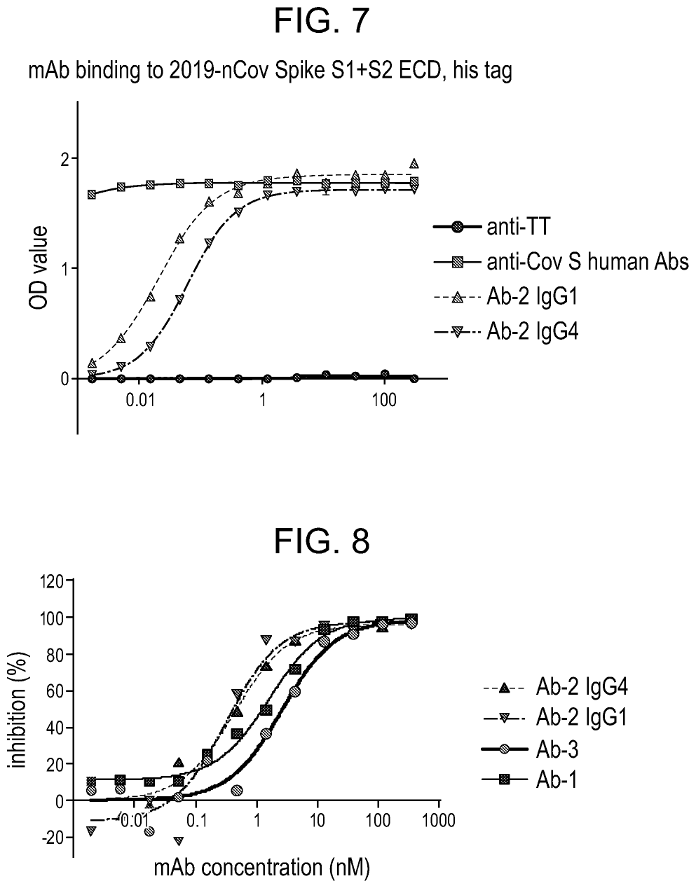

Comparable binding affinity was also verified using Ab-2, either in hIgG1 or hIgG4 format, in ELISA assay. The result in FIG. 7 shows that both formats had similar binding affinity for the His-tagged S1+52 ECD. The measured EC50 for hIgG1 is 0.021 nM, and for hIgG4 is 0.061 nM, though the lower EC50 value for hIgG1 was due to the use of different secondary antibody.

Next, pseudo-virus neutralization activity of the IgG1 vs. IgG4 formats of Ab-2 were compared (data for Ab-1 and Ab-3 were also included). The results were shown in FIG. 8 and summarized in the table below.

| EC50/EC80 or IC50/IC80 (nM) | | Ab-2 IgG1 | Ab-2 IgG4 | Ab-3 | Ab-1 |
|---|---|---|---|---|---|
| Pseudo-virus | IC50 | 0.53 | | 5.80 | 4.07 |
| neutralization (Test 1) | IC80 | 2.11 | | 23.20 | 16.28 |
| Pseudo-virus | IC50 | 0.32 | 0.44 | 2.61 | 1.62 |
| neutralization (Test 2) | IC80 | 1.29 | 1.76 | 10.44 | 6.48 |

Live-virus neutralization activity between IgG1 vs IgG4 formats of Ab-2 were also compared. In one experiment, a fixed amount of live SARS-CoV-2 virus was mixed with about equal volumes of a serial dilution of the IgG4 version of the Ab-2 antibody, before the neutralized or partially neutralized virus was used to infect single layer of Vero E6 cells (2 repeats for each Ab dilution). CPE (cytopathic effect) was observed at 3-5 days post infection in lower dilutions but not in higher dilutions. Experimental results were summarized below.

| Well No. | 1 | 6 | 7 | 8 | 9 | 12 |
|---|---|---|---|---|---|---|
| Dilution fold (1:N) | 10 | 320 | 640 | 1,280 | 2,560 | 20,480 |
| Ab Conc. (μg/mL) | 20 | 0.625 | 0.3125 | 0.15625 | 0.078 | 0.010 |
| Repeat 1 | − | − | 2+ | 3+ | 4+ | 4+ |
| Repeat 2 | − | − | − | 3+ | 4+ | 4+ |

* Serial 2-fold Ab dilution was made from the 1:10 dilution of 200 μg/mL original Ab concentration (z.e., 20 μg/mL) in Well No. 1, until 20,480-fold dilution in Well No. 12
** “−” means normal cell, “+” means 0-25% cells exhibited CPE, “2+” means 26-50% cells exhibited CPE, “3+” means 51-75% cells exhibited CPE, “4+” means 76-100% cells exhibited CPE. Experiment concluded when virus positive control (z.e., 100TCID50 cells) exhibited 3+ to 4+ CPE.
*** Vero E6 cells without virus infection grew normally; positive control antibody had neutralizing activity. Viral TCID50 was 10^{-5.5}.

According to the Reed-Muench method, about 0.448 μg/mL (or about 3 nM) of IgG4 Ab-2 protected about 50% of Vero E6 cells from exhibiting SARS-CoV-2 infection-induced CPE under the experimental condition.

In another experiment similar to FIG. 3A, Ab-2 IgG4-YTE format was found to be as potent as Ab-2 IgG1, and perhaps slightly more potent in this assay (data not shown). Blockage of live virus entry was achieved around 2 nM for IgG4 Ab-2. Meanwhile, as a control, Ab-4 displayed no neutralizing activity at the highest dose tested (300 nM).

Engineering of antibodies for improved pharmacokinetics through enhanced binding to the neonatal Fc receptor (FcRn) has been demonstrated in transgenic mice, non-human primates and humans. Booth et al., “Extending human IgG half-life using structure-guided design,” MAbs, 2018 Oct. 10(7) 1098-1110. Recombinant antibodies' therapeutic potential may be enhanced by the introduction of defined mutations in the crystallizable fragment (Fc) domains, such as for example, YTE (M252Y/S254T/T256E) and LS (M428L/N434S), as a consequence of increased half-lives and prolonged duration of protection. For example, The prototypical example of an FcRn affinity-enhancing Fc mutant is the YTE mutation which, when incorporated into motavizumab IgG1, is able to extend serum half-life in humans by more than four-fold. Robbie et al., A novel investigational Fc-modified humanized monoclonal antibody, motavizumab-YTE, has an extended half-life in healthy adults. Antimicrob Agents Chemother. 2013; 57:6147-6153. Booth et al. and Robbie et al. are incorporated herein.

A series of developability assays were performed for the IgG4 version of Ab-2 and Ab-3, including accelerated stability (2-3 mg/mL of Ab at 25 and 40° C. in D-PBS, pH7.4, for up to 14 days); forced degradation (2-3 mg/mL of Ab at 25° C. in 100 mM acetic acid at pH3.5, for up to 6 hours); and up to 5 freeze-thaw cycles (2-3 mg/mL of Ab). The results showed that all samples were stable in the accelerated stability study; both antibodies showed aggregation formation under the low pH stress condition; and all samples remained stable after 5 cycles of freeze-thaw.

The data provided herein provides a deeper understanding of the immune response mounted by patients recovering from COVID-19, and may give insights into disease epidemiology and inform the development of novel therapies.

For example, despite the documented greater severity of the disease in males as compared to females, males seem to demonstrate higher antiviral titers as a group. This may be related to higher viral loads during the period of peak infection. Several mechanisms have been proposed to explain the apparently greater susceptibility of males to COVID-19, including 1) A high prevalence of smoking among males in regions where the SARS-CoV-2 virus has spread to humans, and 2) the ACE2 gene that acts as a receptor for the virus on human epithelial cells being located on the X chromosome, which may lead to sex-specific differences in ACE2 expression and susceptibility to infection or viral burden. Children have been observed to have less severe disease symptoms as compared to adults (Dong et al., 2020). Interestingly, an 8-year-old patient profiled as part of the cohort studied here showed relatively high titers to the SARS-CoV-2 S protein, suggesting that despite their less mature immune systems, children are capable of mounting a robust antiviral immune response.

COVID-19 patients tend to display low lymphocyte counts, and reduced levels and functional exhaustion of T cells has been described in patients with severe disease (Ni et al., 2020). It is therefore notable to find increased levels of circulating plasma cells as well as memory B cells in convalescent patients weeks after initial infection and days after recovery, suggesting that the humoral immune response is critical in limiting viral activity.

Use of convalescent patient sera as an immediate way to transfer protective immunity to newly diagnosed patients and at-risk populations is a therapeutic strategy being applied to treat COVID-19, and neutralizing antiviral monoclonal antibodies identified from these sera have the utility to be scalable treatments for the disease. Further mining of immune repertoires from patients recovering from COVID-19, using their methods of the invention described herein can be key to controlling the future spread of this and other similar viruses.

The methods described herein identified antibodies binding to SARS-CoV-2 coronavirus, thus permitting further characterization of the neutralizing activities of these antibodies, as well as mapping out binding epitopes of these antibodies. In one embodiment, these neutralizing antibodies can be formulated for use as therapeutic antibodies for patient treatment. In another embodiment, they can also be used prophylactically to prevent virus infection. In further embodiments, certain binding antibodies can be used in combination with the vaccine approaches, even if they do not have neutralization activities. In yet another embodiments, different antibodies with either S1 or S2 binding capacity are used to generate multi-valent antibodies, or be used together, for combination therapy.

### Example 4 Efficacy of Antibodies Against SARS-CoV-2 Variants

### Live Virus Variant Study

Similar to live virus study described above, Vero E6 cells were infected by the WuhanD614, BavPat D614G and UK B.1.1.7 (also named 20I/501Y.V1) SARS-CoV-2 variants and incubated with the subject antibodies with a series of two-fold dilutions in the 0.97-1000 ng/ml range in triplicate. Viral RNA in the supernatant was determined, and % of inhibition was calculated based on infected but untreated control. The results are shown in the table below:

| | Ab-2 IgG1 | PR2/HFB2 | PR3/HFB5 | HFB 3 | HFB 4 | HFB 6 | HFB 7 | Ab-2 IgG4 | HFB 9 |
|---|---|---|---|---|---|---|---|---|---|
| ng/ml | EC50 | EC50 | EC50 | EC50 | EC50 | EC50 | EC50 | EC50 | EC50 |
| WuhanD614 | 101.4 | n.c | >1000 | 169.8 | 21.1 | >1000 | 105.60 | 106.53 | 6.45 |
| BavPat D614G | 131.7 | n.c | 468.70 | 168.0 | 48.9 | >1000 | 154.00 | 115.67 | 19.88 |
| UK B.1.1.7 | 57.8 | >1000 | >1000 | 91.5 | 36.1 | >1000 | 38.12 | 37.23 | 2.66 |

Additional tests were performed to compare the effectiveness of the subject antibody (i.e., Ab-2 IgG4) against the ACE2-Fc fusion, in various variant strains. The results are listed in the table below.

IC50 Values s as SARS-CoV-2 Neutralization
Potency Measurement

| Isolates/IC50s (μg/mL) | ACE2-Fc | Ab-2 IgG4 |
|---|---|---|
| WT | 2.858 | 1.031 |
| UK | 0.5224 | 0.76 |
| CA (B.1.429) | 0.4836 | 0.6516 |
| NY (B.1.526) | 0.5563 | 0.1046 |

### Pseudovirus Variant Study

Next, a pseudotyped lentiviral vector with SARS-CoV-2 S proteins as part of the envelope is constructed to mimic SARS-CoV-2 virus, which can infect target cells expressing hACE2. The neutralizing potency was then deduced by detecting the expression levels of luciferase reporter gene packaged into the lentiviral vector. Three variants of SARS-CoV-2 were tested in this experiment: SARS-CoV-2/Wild-type (WT), SARS-CoV-2/United Kingdom (UK, B.1.1.7 lineage) variant and SARS-CoV-2/South Africa (SA, B.1.351 lineage) variant. Those variants represent commonly isolated SARS-CoV-2 clinical isolates.

Under the current experimental condition, Ab-2 IgG4 showed a significant neutralizing potency against two SARS-CoV-2 variants—WT and UK variant. However, little neutralizing activity was observed against SA variant, indicating that mutations in SARS-CoV-2 Spike from SA variant gained the ability to void the neutralizing antibody tested. On the other hand, Ab-5 showed a significant neutralizing potency against SARS-CoV-2/SA variant (data not shown).

### Live Virus Study Using Ab-2 IgG1 and IgG4

SARS-CoV-2 strains (strain BetaCoV/Wuhan/WIV04/2019) at 100 TCID₅₀ per 50 μL was mixed with equal volume of culture medium containing serially Ab-2 IgG1 and Ab-2 IgG4 diluted antibodies and incubated at 37° C. for 1 hour, and then added to Vero E6 cells seeded in 96-well plates. After 48 hrs of culture at 37° C., cells were fixed and processed for SARS-CoV-2 nucleocapsid protein (NP) and nuclei staining. % Inhibition was calculated by (total nuclei-infected cells)/total nuclei ×100%. Fifty percent neutralization dose (ND₅₀) and ninety percent neutralization dose (ND₉₀) were calculated using 4-parameter non-linear regression with GraphPad Prism 8.0. The experiment was carried out in triplicate.

Ab-2 IgG4 and Ab-2 IgG1 exhibited potent neutralization activities against SARS-CoV-2 live virus infections of Vero E6 cells with ND₅₀ between 2.18-6.5 nM (0.320-0.956 μg/mL) and ND₉₀ between 2.18-11.68 nM (0.320-1.681 μg/mL), respectively.

### Example 5 SARS-CoV-2 S Protein Variant Binding by Ab-2 IgG4

The binding of the Ab-2 IgG4 antibody to SARS-CoV-2 S protein variants (S477N, S494P, F490S, Y453F, N439K, N501Y, E484K, Q493R, and A222V/D614G) was determined by enzyme linked immunosorbent assay (ELISA). The 384-well plates were coated with 20 nM of the different SARS-CoV-2 S protein RBD variants. The binding of Ab-2 IgG4 (12 concentrations obtained by 3-fold serial dilutions starting from 300 nM, in triplicate) to the SARS-CoV-2 S protein RBD (receptor binding domain) variants was detected by goat F(ab′)₂ anti-human IgG (H+L)-HRP. In this experiment, Ab-2 IgG4 exhibited potent binding to all tested SARS-CoV-2 S protein RBD/S1 variants with comparable EC50 values ranging from 0.10-0.37 nM.

The blocking activity of Ab-2 IgG4 was determined by enzyme linked immunosorbent assay (ELISA). The 384-well plates were coated with 20 nM of the hACE2-mFc protein. SARS-CoV-2 S protein RBD/S1 variants (with His tag) at fixed concentration (binding EC90 of corresponding variant to hACE2-mFc) was pre-incubated with Ab-2 IgG4 or isotype control at different concentrations (12 concentrations obtained by 3-fold serial dilutions starting from final concentration of 300 nM, in duplicates), before incubated with coated hACE2-mFc protein. The binding of SARS-CoV-2 S protein RBD/S1 variants (with His tag) to the hACE2-mFc protein was detected by HRP anti-6×His tag antibody. In this experiment, Ab-2 IgG4 blocked the binding of 9 tested SARS-CoV-2 S protein RBD/S1 variants (S477N, S494P, F490S, Y453F, N439K, N501Y, E484K, Q493R, and A222V/D614G) to hACE2-mFc protein with IC50 ranging from 0.6-13.15 nM.

Additionally, binding affinities of Ab-2 IgG4 to WT RBD or 6 mutant RBD variants (Y453F, S477N, S494P, F490S, N439K, N501Y) and a S1 variant (A222V/D614G) were evaluated using SPR (Biacore T200).

Summary of binding affinities* of seven SARS-CoV-2
S protein RBD/S1 variants to Ab-2 IgG4 using SPR

| SARS-CoV-2 S protein | | | |
|---|---|---|---|
| RBD/S1 variant | Ka (1/Ms) | Kd (1/s) | K_{D} (M) |
| WT | 9.565E+4 | 1.706E−4 | 1.784E−9 |
| Y453F | 1.073E+5 | 0.001084 | 1.010E−8 |
| S477N | 1.198E+5 | 2.278E−4 | 1.902E−9 |
| S494P | 7.588E+4 | 1.191E−4 | 1.570E−9 |
| F490S | 1.072E+5 | 4.234E−4 | 3.949E−9 |
| WT | 8.598E+4 | 1.675E−4 | 1.948E−9 |
| N439K | 6.981E+4 | 2.427E−4 | 3.476E−9 |
| N501Y | 7.161E+4 | 6.937E−4 | 9.687E−9 |
| A222V/D614G | 4.178E+4 | 4.249E−5 | 1.017E−9 |

*1:1 binding model was used for evaluating the binding affinity

### Example 6 Absence of Antibody-Dependent Enhancement (ADE) by Ab-2 IgG4

One potential hurdle for antibody-based therapeutics is the risk of exacerbating COVID-19 severity via antibody-dependent enhancement (ADE). ADE has been documented to occur through two distinct mechanisms in viral infections: by enhanced antibody-mediated virus uptake into Fc gamma receptor IIa (FcγRIIa)-expressing phagocytic cells leading to increased viral infection and replication, or by excessive antibody Fc-mediated effector functions or immune complex formation causing enhanced inflammation and immunopathology. Both ADE pathways can occur when non-neutralizing antibodies or antibodies at sub-neutralizing levels bind to viral antigens without blocking or clearing infection.

Raji cells, originally derived from a Burkitt's lymphoma patient, have been shown to facilitate SARS-CoV-1 infection in the presence of anti-S-protein immune serum. Thus, this FcγRII-bearing human B lymphoblast cell line was used to study the antibody-dependent viral entry of SARS-CoV-2 as an indicator of ADE. Briefly, Raji cells were seeded in 96-well plates. Antibodies at different concentrations were pre-incubated with the SARS-CoV-2 pseudo virus encoding wild-type spike protein and luciferase. The mixture of antibody and pseudo virus was then added to plated Raji cells. The plates were incubated and pseudovirus infection of Raji cells was quantified by measuring luciferase activity.

Antibody-dependent entry of SARS-CoV-2 pseudovirus in the presence of a positive control antibody was observed. On the other hand, Ab-2 IgG4 did not show any sign of antibody-dependent enhancement of SARS-CoV-2 pseudovirus infection of Raji cells.

The data suggests that Ab-2 IgG4 does not exacerbate a SARS-CoV-2 infection via ADE.

### Example 7 Pharmacokinetics of Ab-2 IgG4 in Rhesus Monkeys

### Systemic Circulation of Antibody

A total of 9 Rhesus monkeys were included for efficacy study of Ab-2 IgG4. Monkeys were allocated into 3 groups (three per group) receiving a single intravenous infusion of isotype control at 50 mg/kg (group 1), Ab-2 IgG4 at 10 mg/kg (group 2) and Ab-2 IgG4 at 50 mg/kg (group 3) one day after intratracheal inoculation of SARS-CoV-2 at 1×10⁵ TCID₅₀. Serum samples were collected once daily from 0-7 days post infection (d.p.i.). The concentration of Ab-2 IgG4 in plasma samples was determined using validated ELISA methods.

Briefly, plates were coated at 4° C. overnight with anti-human (h) IgG (quantification of total hIgG) or SARS-CoV-2 S protein S1 subunit recombinant protein (quantification of unbound Ab-2 IgG4), before incubated with rhesus monkey plasma collected during Ab-2 IgG4-012 Study. The concentration of plasma antibodies was detected by HRP conjugated anti-hIgG antibody.

Under these experimental conditions, the systemic exposure of Ab-2 IgG4 were achieved in all treated Rhesus monkeys. Ab-2 IgG4 exhibited linear clearance with approximate dose proportionality. The mean AUC_{0-6d} of Ab-2 IgG4 in 10 and 50 mg/kg dosing groups were 222 and 1643 μg/mL*day, respectively, by total hIgG, and 419 and 2398 μg/mL*day, respectively, by unbound Ab-2 IgG4.

### Pharmacokinetics and Immunogenicity Study

A study was conducted to evaluate serum pharmacokinetics (PK) and immunogenicity following a single IV infusion administration of Ab-2 IgG4 in naïve male and female cynomolgus monkeys.

On Day 1 of the study, 3 male and 3 female cynomolgus monkeys were administered a single 10 mg/kg dose of Ab-2 IgG4 by IV infusion (60 minutes; 4 mL/kg). Blood was collected and processed for PK, anti-drug antibody (ADA), hematology, and clinical chemistry evaluations (data not shown). The concentration of Ab-2 IgG4 and ADA, was monitored in serum for up to 56 days (1345 hours) after beginning of infusion and are provided below. Safety was also monitored based on clinical observations and hematology and clinical chemistry evaluations.

The quantification of serum Ab-2 IgG4 concentrations and ADA were conducted at WuXi AppTec Bioanalytical Services Department using a validated ELISA method or a validated electrochemiluminescent (ECL) method, respectively. Serum concentration versus time data was analyzed by non-compartmental model using the WinNonlin software (version 6.3, Pharsight, Mountain View, Calif.).

Individual animal Ab-2 IgG4 serum concentrations over time were measured (not shown) and a summary of the Ab-2 IgG4 PK parameters is provided in the table below. A single dose of 10 mg/kg resulted in a combined (males and females) mean area under the serum drug concentration-time curve up to the last quantifiable time-point 56 days post start of infusion (AUC_{0-1345 h}) of 77,500,000 ng·h/mL and mean maximum observed serum concentration (Cₘₐₓ) of 266,000 ng/mL. The mean time to maximum concentration (Tₘax) was 1.0833 hours post beginning of infusion and the mean terminal half-life (T½) was 459 hours. There were no notable gender-related differences in Ab-2 IgG4 systemic exposures in cynomolgus monkeys (data not shown). From Day 1 through Day 56 of the study, there were no adverse clinical observations, body weight changes, or abnormal hematology and blood chemistry test results.

Summary of Ab-2 IgG4 Pharmacokinetic (PK) Parameters Following a
Single Intravenous Infusion in Male and Female Cynomolgus Monkeys

| | Group | | | |
|---|---|---|---|---|
| | 1 | | | |
| | Dose Route | | | |
| | IV Infusion (60 minutes; 4 mL/kg) | | | |
| | Dose level (mg/kg) | | | |
| | 10 | | | |
| | Male Mean | Female Mean | Combined Mean | |
| PK parameters | (N = 3) | (N = 3) | (N = 6) | SD |
| Cₘₐₓ (ng/mL) | 259000 | 273000 | 266000 | 36200 |
| Tₘₐₓ (h) | 1.0833 | 1.0833 | 1.0833 | 0.00 |
| T_{1/2} (h) | 586 | 332 | 459 | 258 |
| V_{dss} (L/kg) | 0.0747 | 0.0617 | 0.0682 | 0.0130 |
| V_{z} (L/kg) | 0.0732 | 0.0526 | 0.0629 | 0.0236 |
| CL (mL/min/kg) | 0.00151 | 0.00277 | 0.00214 | 0.00132 |
| Group | 1 | | | |
| AUC_{0-1345 h} | 88,200,000 | 66,700,000 | 77,500,000 | 21,900,000 |
| (ng · h/mL) | | | | |
| AUC_{0-inf} | 114,000,000 | 76,100,000 | 95,000,000 | 35,800,000 |
| (ng · h/mL) | | | | |

Abbreviations: AUC_{0-1345 h} = area under the serum drug concentration-time curve up 1345 hours post start of infusion;
AUC_{0-inf} = area under the serum drug concentration-time curve to infinite time;
CL = total body clearance;
Cₘₐₓ = maximum observed serum concentration;
IV = intravenous;
T½ = terminal half-life;
Tₘₐₓ = time to first occurrence of Cmax;
V_{dss} = apparent volume of distribution at steady state;
Vz = apparent volume of distribution during the terminal phase.

### Example 8 In Vivo Efficacy of Ab-2 IgG4 in Rhesus Monkeys

The efficacy of Ab-2 IgG4 to treat SARS-CoV-2 infection was evaluated in Rhesus monkeys. On Day 0, 3 groups of 3 Rhesus monkeys (2 females and 1 male per group) were infected by intratracheal (IT) inoculation with SARS-CoV-2 at 1×10⁵ TCID₅₀/animal. On Day 1, following confirmation of infection via oropharyngeal swab, a single treatment with isotype control antibody (50 mg/kg) or Ab-2 IgG4 at 10 or 50 mg/kg was administered by IV infusion. Health status and infection were monitored via body temperature, body weight, hematology, and blood chemistry analyses from samples collected prior to viral challenge (pi0d) and on Days 1 (blood, swabs, and feces collected before Ab-2 IgG4 administration), 2, 3, 4, 5, 6, and 7. SARS-CoV-2 viral load was evaluated in blood; oropharyngeal, nasal and rectal swabs; and feces using RT-qPCR method. The lungs were X-rayed on Days 0 (prior to infection), 3, and 6. One monkey from each group was euthanized 5, 6 and 7 days post-infection and selected organs (lungs [6 lobes, trachea, left and right bronchia], spleen, pulmonary hilar lymph node, liver, and kidney) were processed, stained with hematoxylin and eosin [H&E] and Masson's trichrome staining, and associated pathological changes were evaluated microscopically. Serum samples were collected prior to viral challenge (pi0d) and on Days 1 (before Ab-2 IgG4 administration), 2, 3, 4, 5, 6, and 7, for evaluation of Ab-2 IgG4 levels. The findings for animal health (body weight, body temperature), viral load, X-rays, and microscopic evaluations of lung tissues have been reported, as well as pharmacokinetics results.

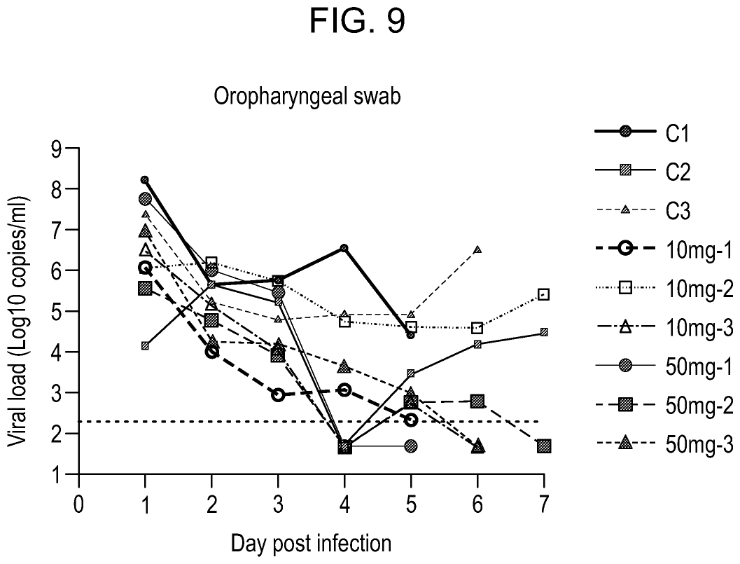

No significant changes in body temperature or body weight were noted throughout the study. A single treatment with Ab-2 IgG4 at 10 or 50 mg/kg markedly decreased detectable SARS-CoV-2 viral load in oropharyngeal swabs by Day 4 (FIG. 9). Antiviral activity was demonstrated in 5 of 6 animals treated with a single dose of Ab-2 IgG4 antibody (FIG. 9): resolution of viral shedding in the oropharynx by the endpoint of the study was observed in all 3 animals dosed with 50 mg/kg of Ab-2 IgG4, and in 2 out of 3 animals dosed with 10 mg/kg Ab-2 IgG4, but in 0 out of 3 animals dosed with isotype control antibody at 50 mg/kg (FIG. 9). Viral load was detected in some of the nasal and rectal swabs only at some of the timepoints (data not shown).

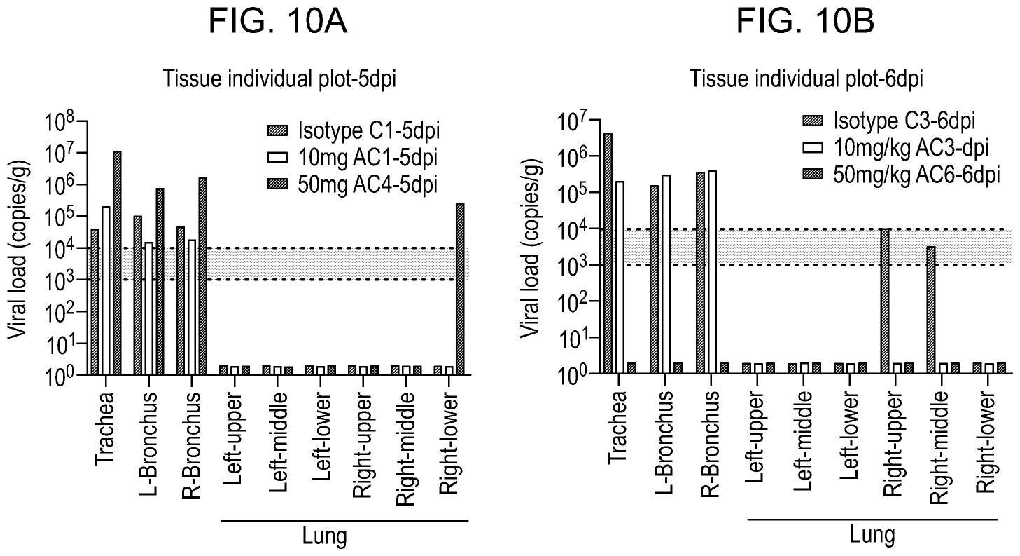

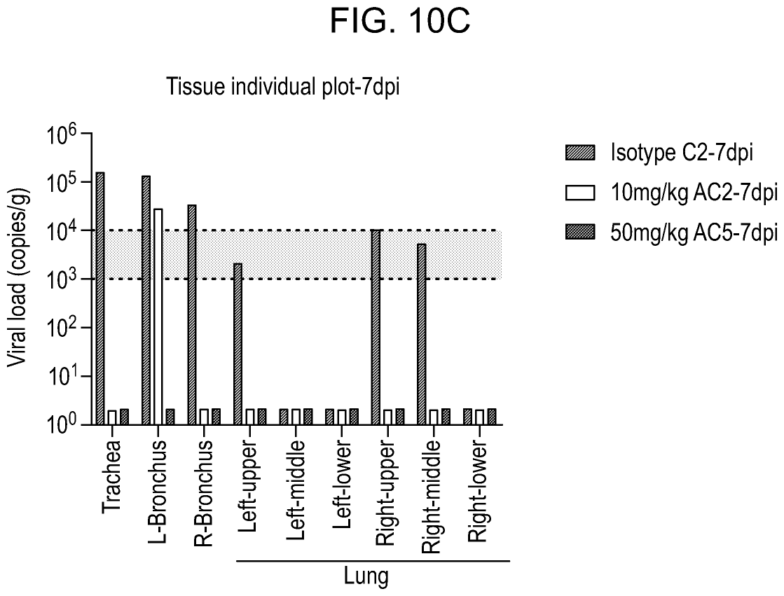

Viral load was also evaluated in different tissues and showed consistently detectable viral RNA (1×10⁴-1×10⁷ viral RNA copies/gram) in all 3 isotype control group animals in the trachea, left and right bronchus, and 2 or 3 of the 6 lung lobes in the 2 animals euthanized on days 6 and 7 post infection (FIGS. 10A-10C). In the Ab-2 IgG4 10 mg/kg group, viral RNA (1×10⁴-1×10⁶ viral RNA copies/gram) was detectable in the trachea, left and right bronchus of the 2 animals euthanized on days 5 and 6 post infection and only in the left bronchus of the animal euthanized 7 days post infection, and was not detected in any of the lungs from all 3 animals (FIGS. 10Error! Reference source not found.A-10C).

In contrast, in Ab-2 IgG4 50 mg/kg treatment group, viral RNA (1×10⁵-1×10⁷ viral RNA copies/gram) was only detected in the trachea, left and right bronchus, and 1 lung lobe in the animal euthanized 5 days post infection, but not in any of the tissues tested from the 2 animals euthanized on day 6 and 7 post infection (FIGS. 10A-10C).

Microscopic evaluation of the lungs (picture not shown) of the animals in the isotype control group or Ab-2 IgG4 10 mg/kg group on Day 7 post infection (6 days after treatment) revealed severe and moderate to severe lung lesions, respectively, characterized by: (1) thickened alveolar walls with large numbers of monocyte and lymphocyte infiltrates, fibroblast hyperplasia, and fibrosis; (2) monocyte and lymphocyte infiltrates, edema, cellulose exudation and/or hyaline membrane formation, fibroblast proliferation, and fibrosis in the alveolar cavities, with compensating emphysema in some alveoli; and (3) pulmonary hemorrhage in the lungs. The lesions generally centered around small bronchial tubes with numerous epithelial cells observed in some small bronchial cavities. Emphysema under the pleural was observed with localized pleural wall thickening and fibrosis on part of the pleural. The lung lesions in the 50 mg/kg Ab-2 IgG4 treatment group were reduced to mild-moderate with most of the alveoli displaying normal structure. A summary comparison of lung lesions in different animals is shown in the following table.

Summary Comparison of Pulmonary Lesions in SARS-CoV-2 Rhesus Monkeys After
a Single IV Treatment with Isotype Control Antibody or Ab-2 IgG4

| | | Day 5. | | | | | | Day 6. | | |
|---|---|---|---|---|---|---|---|---|---|---|
| | | Inflam- | | Inflam- | | Inflam- | Inflam- | Inflam- | | Inflam- |
| | | matory | | matory | | matory | matory | matory | | matory |
| | | cell | Thickening.of | leakage in | Alveolar | cells around | cells around | cell | Thickening.of | leakage in |
| | Animal | infiltration | alveoli | alveolar | cavity | small | blood | infiltration | alveoli | alveolar |
| Group | number | in alveoli | walls | cavity | fibrosis | bronchial | vessels | in alveoli | walls | cavity |
| Isotype | C1 | ++ | ++ | ++ | + | + | − | / | / | / |
| control | C2 | / | / | / | / | / | / | / | / | / |
| | C3 | / | / | / | / | / | / | ++ | ++ | + |
| | Total | ++ (Moderate) | | | | | | ++ (Moderate) | | |
| Low-dose | AC1 | ++ | ++ | + | ++ | + | − | / | / | / |
| treatment | AC2 | / | / | / | / | / | / | / | / | / |
| group | AC3 | / | / | / | / | / | / | ++ | ++ | ++ |
| | Total | ++ (Moderate) | | | | | | ++ (Moderate) | | |
| High-dose | AC4 | ++ | ++ | + | + | + | − | / | / | / |
| treatment | AC5 | / | / | / | / | / | / | / | | / |
| group | AC6 | / | / | / | / | / | / | + | + | + |

| | | |
|---|---|---|
| Total | +/++ (Mild-moderate) | + (Mild) |

| | | Day 6. | | | Day 7. | | | | | |
|---|---|---|---|---|---|---|---|---|---|---|
| | | | Inflam- | Inflam- | Inflam- | | Inflam- | | Inflam- | Inflam- |
| | | | matory | matory | matory | | matory | | matory | matory |
| | | Alveolar | cells around | cells around | cell | Thickening.of | leakage in | Alveolar | cells around | cells around |
| | Animal | cavity | small | blood | infiltration | alveoli | alveolar | cavity | small | blood |
| Group | number | fibrosis | bronchial | vessels | in aveoli | walls | cavity | fibrosis | bronchial | vessels |
| Isotype | C1 | / | / | / | / | / | / | / | / | / |
| control | C2 | / | / | / | +++ | +++ | ++ | ++ | + | + |
| | C3 | + | + | + | / | / | / | / | / | / |
| | Total | ++ (Moderate) | | | +++ (Severe) | | | | | |
| Low-dose | AC1 | / | / | / | / | / | / | / | / | / |
| treatment | AC2 | / | / | / | ++ | +++ | ++ | ++ | + | − |
| group | AC3 | + | + | − | / | / | / | / | / | / |
| | Total | ++ (Moderate) | | | ++/+++ (Moderate-Severe) | | | | | |
| High-dose | AC4 | / | / | / | / | / | / | / | / | / |
| treatment | AC5 | / | / | / | ++ | ++ | + | + | + | + |
| group | AC6 | + | + | − | / | / | / | / | / | / |

| | | |
|---|---|---|
| Total | + (Mild) | +/++ (Mild - Moderate) |

The serum drug concentration was determined using 2 optimized ELISA methods measuring total hIgG or unbound Ab-2 IgG4. After intravenous administration of Ab-2 IgG4, serum exposure was observed in all animals. Ab-2 IgG4 exhibited linear clearance with approximate dose proportionality. The mean AUC_{0-6days} of Ab-2 IgG4 in 10 and 50 mg/kg dosing groups were 222 and 1643 μg/mL*day, respectively, by total hIgG, and 419 and 2398 μg/mL*day, respectively, by unbound Ab-2 IgG4 (see Table below).

| | Group 2 | Group 3 | | |
|---|---|---|---|---|
| | Ab-2 IgG4 | Ab-2 IgG4 | | |
| | 10 mg/kg | 50 mg/kg | Group 2 vs Group 3 | |
| | AUC_{0-6 d} | AUC_{0-6 d} | Dose | AUC_{0-6 d} |
| ELISA method | (μg/mL*day) | (μg/mL*day) | ratio | ratio |
| Total hIgG | 222.36 | 1642.98 | 1:5 | 1:7.39 |
| Unbound Ab-2 IgG4 | 419.36 | 2397.65 | 1:5 | 1:5.72 |

In conclusion, intra-tracheal inoculation of SARS-CoV-2 (1×10⁵ TCID⁵⁰) in Rhesus monkeys resulted in viral replication in the respiratory tract and lung lesions in isotype control (50 mg/kg) treated animals.

In contrast to the treatment with isotype control antibody, Ab-2 IgG4 effectively inhibited viral replication, as shown by a progressive declining of viral loads in the oropharyngeal swabs of animals treated with a single dose of 10 mg/kg or 50 mg/kg Ab-2 IgG4 one day after viral inoculation (2 of 3 animals at 10 mg/kg and 3 of 3 animals at 50 mg/kg) and decreased viral distribution in respiratory tract and lungs.

Based on lung imaging and microscopic analysis, isotype control treated animals exhibited pathological changes including moderate to severe lesions characterized by thickened alveoli walls, fibroblast hyperplasia, fibrosis, large numbers of monocyte and lymphocyte infiltrates, alveoli edema with small amounts of cellulose exudate and/or hyaline membrane formation in alveolar cavities, and pulmonary hemorrhage. The lung lesions generally centered around small bronchial tubes with numerous epithelial cells observed in some small bronchial cavities. Emphysema under the pleural was observed with localized pleural wall thickening and fibrosis on part of the pleural.

Although similar microscopic changes were noted in the lungs in all groups, the severity was reduced to moderate in the 10 mg/kg Ab-2 IgG4 treatment group, and mild-moderate in the 50 mg/kg Ab-2 IgG4 treatment group with most of the alveoli displaying normal structure. After intravenous administration of Ab-2 IgG4, serum exposure was observed in all animals. Ab-2 IgG4 exhibited linear clearance with approximate dose proportionality.

Together, these data demonstrate the antiviral activity of Ab-2 IgG4 for the treatment of SARS-CoV-2 in a Rhesus monkey infection model.

### Example 9 Toxicology Study

The purpose of this study was to determine the potential toxicity of Ab-2 IgG4 when administered by intravenous infusion at 0, 50 and 300 mg/kg/dose to cynomolgus monkey once weekly for 14 days (two doses in total) and to assess the reversibility, persistence, or delayed occurrence of potential toxic effects following a 56-day recovery phase. In addition, the toxicokinetics (TK) of Ab-2 IgG4 were determined.

Thirty (15 per sex) cynomolgus monkeys were randomly assigned to 3 groups of 5/sex/group and given Ab-2 IgG4 at 0, 50, or 300 mg/kg/dose once weekly for 2 doses by intravenous (IV) infusion at a dosing volume of 10 mL/kg and dosing rate of 6.7 mL/kg/hour. The infusion duration was 1.5 hours. At initiation of dosing, male and female cynomolgus monkeys were approximately 3 to 5 years of age, and their body weights ranged from 2.2 to 3.6 kg for females and 2.3 to 5.2 kg for males. The dosing phase animals were necropsied on Day 15 and recovery animals were necropsied on Day 71.

Parameters evaluated during the study included viability (morbidity and mortality), clinical observations including tolerance at injection sites, body weight, body weight changes, qualitative food consumption, ophthalmology, body temperature, safety pharmacology (electrocardiography, blood pressure, respiration and neurological examinations), clinical pathology (hematology, serum chemistry, coagulation and urinalysis), toxicokinetics (TK), immunogenicity analysis (anti-drug-antibody), cytokine analysis (IL-2, IL-4, IL-5, IL-6, IL-10, IL-13, TNF-α and IFN-7), gross pathology, organ weights, and histopathology.

Median Tₘₐₓ values for Ab-2 IgG4 were observed between 1.6 and 5.5 hours post start of infusion. Mean or median T_{1/2} values were estimated between 167.0 and 350.3 hours. No marked sex differences in systemic exposure were observed at any dose level. The systemic exposure increased dose-proportionally in males and females on Days 1 and 8 as the dosage increased from 50 to 300 mg/kg. No marked drug accumulation was observed at either dose level. Relatively higher titers of anti-Ab-2 IgG4 antibodies were detected in one male and female given 300 mg/kg/dose during the recovery phase, which resulted in a slight decrease of Ab-2 IgG4 concentrations in these two animals during the recovery phase and has no impact on overall TK evaluation of the study.

There were no animals showing moribund or found dead during the study. All animals survived to scheduled termination. There were no test article-related clinical signs, or effects on body weight, appetite, or findings in ophthalmologic examinations, body temperature, safety pharmacology parameters (quantitative and qualitative electrocardiography, cardiovascular, respiration and neurological changes), hematology, coagulation, urinalysis, serum cytokine (IL-2, IL-4, IL-5, IL-6, IL-10, IL-13, TNF-α, and IFN-7) concentrations, organ weight changes, or macroscopic and microscopic findings. Toward the end of the dosing phase, increased serum globulin (GLB) was noted in males and females given 300 mg/kg/dose, which was likely related to the presence of the test article in the blood, as the test article itself is a monoclonal antibody. Increased serum GLB was not apparent toward the end of the recovery phase.

In conclusion, administration of Ab-2 IgG4 to cynomolgus monkeys once weekly for 2 doses by intravenous infusion at dosages of 0, 50, or 300 mg/kg/dose followed by a 56-day recovery period was well tolerated and did not result in any adverse changes. The no observed adverse effect level (NOAEL) for this study was considered to be 300 mg/kg/dose, the highest dose tested. The corresponding Cₘₐₓ and AUC_{0-169.5h} of Ab-2 IgG4 following the last dose at the NOAEL were 9,880,000 ng/mL and 949,000,000 h*ng/mL for males, and 9,470,000 ng/mL and 916,000,000 h*ng/mL for females, respectively.

### Example 10 Ab-2 Epitope Mapping

Epitope mapping for Ab-2 was conducted to determine the specific residues on the SARS-CoV-2 spike (S) protein that may be involved in binding to Ab-2. Meanwhile, the specific residues on the SARS-CoV-2 spike (S) protein that may be responsible for ACE2 binding and host cell entry were also determined.

Specifically, based on the 3-D crystallography structure of the Ab-2/S protein complex, a first set of S protein residues within 4A of Ab-2 were labeled, and based on the 3-D crystallography structure of the ACE2/S protein complex, a second set of S protein residues within 4 Å of the ACE2 receptor were also labeled. Common residues between the first and the second sets were noted.

It is apparent that among the 17 S protein residues that are within 4 Å of the ACE2 receptor (which are likely involved in ACE2 receptor binding and host cell entry), 15 of these residues are also within 4 Å of Ab-2. This suggests that Ab-2 binding to the Spike protein S will almost completely block off S protein binding to ACE2 receptor, thus preventing SARS-CoV-2 infection of host cells.

The partial protein sequence of the SARS-CoV-2 Wuhan-Hu-1 strain sequence including residues for ACE2 receptor binding and/or Ab-2 binding, from N334 to P527, is shown below and in FIG. 11. The Ab-2 binding epitope residues on the S protein are double underlined, and those of which that are also involved in ACE2 receptor binding are also in bold. The two residues required for ACE2 binding but not Ab-2 binding, G446 and Y449, are in bold and italic font.

Thus the S protein epitope required for Ab-2 binding includes residues T415, G416, K417, D420, Y421, Y453, L455, F456, R457, K458, N460, Y473, Q474, A475, G476, S477, F486, N487, Y489, Q493, S494, Y495, G496, Q498, T500, N501, G502, and Y505 (bold residues are also involved in ACE2 binding).

NLCPFGEVFNATRFASVYAWNRKRISNCVADYSVLYNSASFSTFKCYGV
SPTKLNDLCFTNVYADSFVIRGDEVRQIAPGQ [symbol] IADYNYKLPDDFTG
CVIAWNSNNLDSKV [symbol] GN [symbol] NYL [symbol] R [symbol] SNLKPFERDISTEI [symbol] GS
TPCNGVEG [symbol] C [symbol] FPL [symbol] F [symbol] P [symbol] VG [symbol] QPYRVVVLSFELLHAPAT
VCGP

### Materials and Methods

Antibody Binding and Competition with Receptor ACE2:

The binding affinity of antibodies to spike protein was analyzed by ELISA. 384 well plate (Corning #3700), was coated overnight at 4° C. with PBS containing 30 μL 20 nM of the SARS-CoV-2 Spike S1+S2 ECD, his Tag protein. The next day the plate was washed 5 times with washing buffer (PBS and 0.05% Tween) and then incubated 1 hour at room temperature in blocking buffer (PBS with 2% BSA). After 5 washes the plate was incubated with serial dilution of purified mAbs for 1 hour at room temperature. The plates were then washed 5 times and incubated for 1 hour in detection reagent (Mouse anti-Human IgG Fc HRP labeled (Thermo Fisher 05-4220) at 0.2 μg/ml in 1×PBS with 0.05% Tween and 1% BSA) for 1 hour at room temperature. Following this the plate was washed again 5 times and developed in TMB substrate for 5 min before stopping the reaction with the stop solution. The OD values were determined using Thermo MultiSkan or MD SpectraMax i3X at 450 nm wavelength.

The blocking with receptor ACE2 was performed using cell surface expressed ACE2. 10 nM SARS-CoV-2 Spike S1, mFc tag spike protein was incubated with serial dilution of purified mAbs at room temperature for 1 h and then added to Vero E6 cells (approximately 10⁵ per well) in duplicate. Then detection reagent rabbit anti mouse IgG Fc-AF647 was used. Half-maximal inhibitory concentration (IC50) of the evaluated mAbs were determined with Beckman Cytoflex and FlowJo software analysis.

### Antibody Neutralization Activity Against Pseudo Virus

Murine leukemia virus-based SARS-CoV-2 S pseudotyped virus were prepared by GenScript as previous described. Neutralization assay were performed by incubating pseudo virus with serial dilution of purified antibodies at room temperature for 1 h. ACE2 overexpression Hela cells (approximately 8×10⁴ per well) were cultured in DMEM containing 10% FBS, 1 μg/mL puromycin were added in triplicate into virus-antibody mixture. Following infection at 37° C., 5% CO₂ for 48 h, half-maximal inhibitory concentration (IC₅₀) were determined by luciferase activity using Promega Bio-Glo luciferase assay system with GraphPad Prism.

### Live Virus Assay

Vero E6 cells infected by SARS-CoV-2 at 100 50% tissue-culture infectious doses (TCID₅₀) or infected with a mixture of viruses and diluted antibodies (incubated together for 1 hr) were cultured at 37° C. for 48 hours, and then fixed with 4% paraformaldehyde diluted in PBS (pH=7.2) for 15 minutes at room temperature and then penetrated with 0.25% triton-X 100 for 10-15 minutes. After three washes, cells were blocked at 37° C. for 1 hour using PBS containing 5% BSA, then incubated with in-house prepared anti-SARS-CoV-2 NP rabbit serum as primary antibody and FITC or Alexa Fluor® 488-conjugated goat anti-mouse IgG antibody as the secondary antibody. Cell nuclei were stained using Hoechst 33258 at room temperature for 10 minutes. Images were taken under an inverted fluorescence microscope (Nikon). In some experiments, numbers of nuclei and cells infected with viruses were counted using an Operetta CLSTM system. % inhibition was calculated by (total nuclei-infected cells)/(total nuclei)×100%. Fifty percent neutralization dose (ND50) and ninety percent neutralization dose (ND90) were calculated using 4-parameter non-linear regression with GraphPad Prism 8.0.

### REFERENCES

Bornholdt, Z. A., Turner, H. L., Murin, C. D., Li, W., Sok, D., Souders, C. A., . . . Walker, L. M. (2016). Isolation of potent neutralizing antibodies from a survivor of the 2014 Ebola virus outbreak. Science, 351(6277), 1078-1083. https://doi.org/10.1126/science.aad5788
Casadevall, A., & Pirofski, L.-A. (2020). The convalescent sera option for containing COVID-19. The Journal of Clinical Investigation, 2. https://doi.org/10.1172/JCI138003
Diao, B., Wang, C., Tan, Y., Chen, X., Liu, Y., Ning, L., . . . Chen, Y. (2020). Reduction and Functional Exhaustion of T Cells in Patients with Coronavirus Disease 2019 (COVID-19). MedRxiv, 2020.02.18.20024364. https://doi.org/10.1101/2020.02.18.20024364
Dong, Y., Mo, X., Hu, Y., Qi, X., Jiang, F., Jiang, Z., & Tong, S. (2020). Epidemiological Characteristics of 2143 Pediatric Patients With 2019 Coronavirus Disease in China. Pediatrics. https://doi.org/10.1542/peds.2020-0702
Eyer, K., Doineau, R. C. L., Castrillon, C. E., Briseno-Roa, L., Menrath, V., Mottet, G., . . . Baudry, J. (2017). Single-cell deep phenotyping of IgG-secreting cells for high-resolution immune monitoring. Nature Microystems and Nanoengineering, 1-10. Retrieved from http://dx.doi.org/10.1038/nbt.3964
Gérard, A. et al. (2020). High-throughput single-cell activity-based screening and sequencing of antibodies using droplet microfluidics. Nature Biotechnology, 38, 715-721.
Lavinder, J. J., Horton, A. P., Georgiou, G., & Ippolito, G. C. (2015). Next-generation sequencing and protein mass spectrometry for the comprehensive analysis of human cellular and serum antibody repertoires. Current Opinion in Chemical Biology, 24, 112-120. https://doi.org/10.1016/j.cbpa.2014.11.007
Lee, W. S., Wheatley, A. K., Kent, S. J. et al (2020). Antibody-dependent enhancement and SARS-CoV-2 vaccines and therapies. Nat Microbiol 5, 1185-1191. https://doi.org/10.1038/s41564-020-00789-5
Li, F. (2016). Structure, Function, and Evolution of Coronavirus Spike Proteins. Annual Review of Virology, 3(1), 237-261. https://doi.org/10.1146/annurev-virology-110615-042301
Ni, L., Ye, F., Chen, M.-L., Feng, Y., Deng, Y.-Q., Zhao, H., . . . Dong, C. (2020). Characterization of anti-viral immunity in recovered individuals infected by SARS-CoV-2. MedRxiv, 2020.03.17.20036640. https://doi.org/10.1101/2020.03.17.20036640
Sajadi, M. M., Dashti, A., Rikhtegaran Tehrani, Z., Tolbert, W. D., Seaman, M. S., Ouyang, X., . . . DeVico, A. L. (2018). Identification of Near-Pan-neutralizing Antibodies against HIV-1 by Deconvolution of Plasma Humoral Responses. Cell, 173(7), 1783-1795.e14. https://doi.org/10.1016/j.cell.2018.03.061
Tian, X., Li, C., Huang, A., Xia, S., Lu, S., Shi, Z., . . . Ying, T. (2020). Potent binding of 2019 novel coronavirus spike protein by a SARS coronavirus-specific human monoclonal antibody. Emerging Microbes and Infections, 9(1), 382-385. https://doi.org/10.1080/22221751.2020.1729069
Wrapp, D., Wang, N., Corbett, K. S., Goldsmith, J. A., Hsieh, C.-L., Abiona, O., . . . McLellan, J. S. (2020). Cryo-EM structure of the 2019-nCoV spike in the prefusion conformation. Science, 367(6483), 1260 LP-1263. https://doi.org/10.1126/science.abb2507
Zhou, P., Yang, X.-L., Wang, X.-G., Hu, B., Zhang, L., Zhang, W., . . . Shi, Z.-L. (2020). Discovery of a novel coronavirus associated with the recent pneumonia outbreak in humans and its potential bat origin. BioRxiv, 2020.01.22.914952. https://doi.org/10.1101/2020.01.22.914952
Zhu, N., Zhang, D., Wang, W., Li, X., Yang, B., Song, J., . . . Tan, W. (2020). A novel coronavirus from patients with pneumonia in China, 2019. New England Journal of Medicine, 382(8), 727-733. https://doi.org/10.1056/NEJMoa2001017.

All references are incorporated herein by reference.

What is claimed is:

1. An isolated or recombinantly produced monoclonal antibody, or an antigen-binding fragment thereof, wherein said monoclonal antibody or antigen-binding fragment thereof is specific for an antigen (e.g., the Spike or S protein responsible for ACE2 binding) of SARS-CoV-2, and wherein said monoclonal antibody comprises:
(1a) a heavy chain variable region (HCVR), comprising a HCVR CDR1 sequence of SEQ ID NO: 11, a HCVR CDR2 sequence of SEQ ID NO: 12, and a HCVR CDR3 sequence of SEQ ID NO: 13; and,
(1b) a light chain variable region (LCVR), comprising a LCVR CDR1 sequence of SEQ ID NO: 14, a LCVR CDR2 sequence of SEQ ID NO: 15, and a LCVR CDR3 sequence of SEQ ID NO: 16; or
(2a) a heavy chain variable region (HCVR), comprising a HCVR CDR1 sequence of SEQ ID NO: 1, a HCVR CDR2 sequence of SEQ ID NO: 2, and a HCVR CDR3 sequence of SEQ ID NO: 3; and,
(2b) a light chain variable region (LCVR), comprising a LCVR CDR1 sequence of SEQ ID NO: 4, a LCVR CDR2 sequence of SEQ ID NO: 5, and a LCVR CDR3 sequence of SEQ ID NO: 6; or
(3a) a heavy chain variable region (HCVR), comprising a HCVR CDR1 sequence of SEQ ID NO: 21, a HCVR CDR2 sequence of SEQ ID NO: 22, and a HCVR CDR3 sequence of SEQ ID NO: 23; and,
(3b) a light chain variable region (LCVR), comprising a LCVR CDR1 sequence of SEQ ID NO: 24, a LCVR CDR2 sequence of SEQ ID NO: 25, and a LCVR CDR3 sequence of SEQ ID NO: 26; or
(4a) a heavy chain variable region (HCVR), comprising a HCVR CDR1 sequence of SEQ ID NO: 31, a HCVR CDR2 sequence of SEQ ID NO: 32, and a HCVR CDR3 sequence of SEQ ID NO: 33; and,
(4b) a light chain variable region (LCVR), comprising a LCVR CDR1 sequence of SEQ ID NO: 34 or SEQ ID NO: 115, a LCVR CDR2 sequence of SEQ ID NO: 35, and a LCVR CDR3 sequence of SEQ ID NO: 36; or
(5a) a heavy chain variable region (HCVR), comprising a HCVR CDR1 sequence of SEQ ID NO: 51, a HCVR CDR2 sequence of SEQ ID NO: 52, and a HCVR CDR3 sequence of SEQ ID NO: 53; and,
(5b) a light chain variable region (LCVR), comprising a LCVR CDR1 sequence of SEQ ID NO: 54, a LCVR CDR2 sequence of SEQ ID NO: 55, and a LCVR CDR3 sequence of SEQ ID NO: 56; or
(6a) a heavy chain variable region (HCVR), comprising a HCVR CDR1 sequence of SEQ ID NO: 61, a HCVR CDR2 sequence of SEQ ID NO: 62, and a HCVR CDR3 sequence of SEQ ID NO: 63; and,
(6b) a light chain variable region (LCVR), comprising a LCVR CDR1 sequence of SEQ ID NO: 64, a LCVR CDR2 sequence of SEQ ID NO: 65, and a LCVR CDR3 sequence of SEQ ID NO: 66; or
(7a) a heavy chain variable region (HCVR), comprising a HCVR CDR1 sequence of SEQ ID NO: 71, a HCVR CDR2 sequence of SEQ ID NO: 72, and a HCVR CDR3 sequence of SEQ ID NO: 73; and,
(7b) a light chain variable region (LCVR), comprising a LCVR CDR1 sequence of SEQ ID NO: 74, a LCVR CDR2 sequence of SEQ ID NO: 75, and a LCVR CDR3 sequence of SEQ ID NO: 76; or
(8a) a heavy chain variable region (HCVR), comprising a HCVR CDR1 sequence of SEQ ID NO: 81, a HCVR CDR2 sequence of SEQ ID NO: 82, and a HCVR CDR3 sequence of SEQ ID NO: 83; and,
(8b) a light chain variable region (LCVR), comprising a LCVR CDR1 sequence of SEQ ID NO: 84, a LCVR CDR2 sequence of SEQ ID NO: 85, and a LCVR CDR3 sequence of SEQ ID NO: 86; or
(9a) a heavy chain variable region (HCVR), comprising a HCVR CDR1 sequence of SEQ ID NO: 91, a HCVR CDR2 sequence of SEQ ID NO: 92, and a HCVR CDR3 sequence of SEQ ID NO: 93; and,
(9b) a light chain variable region (LCVR), comprising a LCVR CDR1 sequence of SEQ ID NO: 94, a LCVR CDR2 sequence of SEQ ID NO: 95, and a LCVR CDR3 sequence of SEQ ID NO: 96;
optionally, said isolated monoclonal antibody is not naturally occurring; and/or,
optionally further comprising a signal peptide sequence of SEQ ID NO: 41 at the N-terminus of said HCVR and/or LCVR.

2. The isolated monoclonal antibody or antigen-binding fragment thereof of claim 1, wherein:
(1A) the HCVR sequence is SEQ ID NO: 17; and/or,
(1B) the LCVR sequence is SEQ ID NO: 18, or,
(2A) the HCVR sequence is SEQ ID NO: 7; and/or,
(2B) the LCVR sequence is SEQ ID NO: 8, or,
(3A) the HCVR sequence is SEQ ID NO: 27; and/or,
(3B) the LCVR sequence is SEQ ID NO: 28, or,
(4A) the HCVR sequence is SEQ ID NO: 37; and/or,
(4B) the LCVR sequence is SEQ ID NO: 38 or SEQ ID NO: 114, or,
(5A) the HCVR sequence is SEQ ID NO: 57; and/or,
(5B) the LCVR sequence is SEQ ID NO: 58, or,
(6A) the HCVR sequence is SEQ ID NO: 67; and/or,
(6B) the LCVR sequence is SEQ ID NO: 68, or,
(7A) the HCVR sequence is SEQ ID NO: 77; and/or,
(7B) the LCVR sequence is SEQ ID NO: 78, or,
(8A) the HCVR sequence is SEQ ID NO: 87; and/or,
(8B) the LCVR sequence is SEQ ID NO: 88, or,
(9A) the HCVR sequence is SEQ ID NO: 97; and/or,
(9B) the LCVR sequence is SEQ ID NO: 98.

3. The isolated monoclonal antibody or antigen-binding fragment thereof according to claim 1 or 2, wherein said monoclonal antibody has:
(1a) a heavy chain sequence of SEQ ID NO: 102; and/or,
(1b) a light chain sequence of SEQ ID NO: 20, or,
(2a) a heavy chain sequence of SEQ ID NO: 101; and/or,
(2b) a light chain sequence of SEQ ID NO: 10, or,
(3a) a heavy chain sequence of SEQ ID NO: 103; and/or,
(3b) a light chain sequence of SEQ ID NO: 30, or,
(4a) a heavy chain sequence of SEQ ID NO: 104; and/or,
(4b) a light chain sequence of SEQ ID NO: 40 or SEQ ID NO: 113.

4. The isolated monoclonal antibody or antigen-binding fragment thereof according to any one of claims 1-3, wherein:
(a) the isolated monoclonal antibody is a human antibody, a CDR-grafted antibody, or a resurfaced antibody; and/or,
(b) the antigen-binding fragment thereof is an Fab, Fab′, F(ab′)₂, F_{d}, single chain Fv or scFv, disulfide linked Fᵥ, V-NAR domain, IgNar, intrabody, IgGACH₂, minibody, F(ab′)₃, tetrabody, triabody, diabody, single-domain antibody, DVD-Ig, Fcab, mAb₂, (scFv)₂, or scFv-Fc.

5. The isolated monoclonal antibody or antigen-binding fragment thereof of any one of claims 1-4, wherein said monoclonal antibody or antigen-binding fragment thereof:
(i) binds to the S1 or S2 glycoprotein of SARS-CoV-2;
(ii) binds the SARS-CoV-2 antigen with a K_{d} of less than about 5 nM, 2 nM, 1 nM, 0.5 nM, 0.2 nM, 0.1 nM, or 0.05 nM;
(iii) binds to SARS-CoV-2 wild-type S protein and/or RBD/S1 variants selected from the group consisting of S477N, S494P, F490S, Y453F, N439K, N501Y, E484K, Q493R, and A222V/D614G; and/or,
(iv) inhibits binding of the SARS-CoV-2 antigen (e.g., the S1 glycoprotein) to ACE2, optionally inhibits binding of the SARS-CoV-2 antigen (e.g., the S1 glycoprotein) to ACE2 immobilized on a solid support (such as in ELISA assay), and/or optionally inhibits binding of the SARS-CoV-2 antigen (e.g., the S1 glycoprotein) to ACE2 expressed on the surface of a cell (such as Vero E6 cell).

6. The isolated monoclonal antibody or antigen-binding fragment thereof of any one of claims 1-5, which:
(i) inhibits binding of the SARS-CoV-2 antigen (e.g., the S1 glycoprotein) to ACE2 with an EC50 value of less than 1 nM or 0.1 nM;
(ii) exhibits neutralizing activity against a pseudovirus of SARS-CoV-2 or a live SARS-CoV-2 virus with an IC50 value of less than 10 nM, 6 nM, 3 nM, 1 nM, 0.6 nM or less than 0.5 nM;
(iii) inhibits SARS-CoV-2 viral entry of a target cell (such as Vero E6 cell) at less than 10 nM, 5 nM, 3 nM, 2 nM, 1 nM, 0.1 nM, 0.08 nM, 0.06 nM, 0.02 nM, or less than 0.01 nM;
(iv) inhibits SARS-CoV-2 viral entry of a target cell (such as Vero E6 cell) with an IC50 of less than 10 nM, less than 5 nM, less than 3 nM, less than 2 nM, less than 1 nM, less than 500 pM, less than 200 pM, less than 100 pM, less than 80 pM, less than 50 pM, less than 30 pM, less than 10 pM, or less than 5 pM;
(v) inhibits entry of wild-type SARS-CoV-2, and/or SARS-CoV-2 variants (e.g., WuhanD614, BavPat D614G, UK B.1.1.7, or South Africa B.1.351 strain, or a SARS-CoV-2 variant sharing one or more S1 protein mutations with the WuhanD614, BavPat D614G, UK B.1.1.7, and/or South Africa B.1.351 strain(s)) into a target cell; and/or,
(vi) does not cause antibody-dependent enhancement (ADE).

7. The monoclonal antibody or antigen-binding fragment thereof of any one of claims 1-6, comprising a heavy chain constant region, wherein the heavy chain constant region is human IgG4, human IgG3 or human IgG2; optionally, the heavy chain constant region is human IgG4.

8. An isolated or recombinantly produced monoclonal antibody, or an antigen-binding fragment thereof, wherein said monoclonal antibody or antigen-binding fragment thereof is specific for an antigen (e.g., the S protein responsible for ACE2 binding) of SARS-CoV-2, and wherein said monoclonal antibody comprises a heavy chain variable region (HCVR) comprising a HCVR CDR1 sequence of SEQ ID NO: 11, a HCVR CDR2 sequence of SEQ ID NO: 12, and a HCVR CDR3 sequence of SEQ ID NO: 13, and a light chain variable region (LCVR) comprising a LCVR CDR1 sequence of SEQ ID NO: 14, a LCVR CDR2 sequence of SEQ ID NO: 15, and a LCVR CDR3 sequence of SEQ ID NO: 16,
optionally, the monoclonal antibody or antigen-binding fragment thereof comprises:
(i) an HCVR sequence of SEQ ID NO: 17 or having at least 90%, 91%, 92%, 93%, 94%, 95%, 96%, 97%, 98%, 99% sequence identity to SEQ ID NO: 17; and
(ii) an LCVR sequence of SEQ ID NO: 18 or having at least 90%, 91%, 92%, 93%, 94%, 95%, 96%, 97%, 98%, 99% sequence identity to SEQ ID NO: 18; and/or,
optionally, the monoclonal antibody or antigen-binding fragment thereof comprises a heavy chain constant region of human IgG4, human IgG3, or human IgG2, preferably human IgG4.

9. The monoclonal antibody or antigen-binding fragment thereof of claim 8, comprising a heavy chain (HC) sequence of SEQ ID NO: 102 or having at least 90%, 91%, 92%, 93%, 94%, 95%, 96%, 97%, 98%, 99% sequence identity to SEQ ID NO: 102.

10. An isolated monoclonal antibody or an antigen-binding fragment thereof, which competes with the isolated monoclonal antibody or antigen-binding fragment thereof of any one of claims 1-9 for binding to the same epitope.

11. A mixture of two or more isolated monoclonal antibody or antigen-binding fragment thereof of any one of claims 1-9, optionally, the proportion of each of said two or more isolated monoclonal antibody or antigen-binding fragment thereof is substantially the same, or is different.

12. A polynucleotide encoding the heavy chain and/or the light chain, or the antigen-binding portion thereof, of any one of claims 1-10,
optionally, the polynucleotide is codon optimized for expression in a human cell; and/or,
optionally, the polynucleotide is in a vector, such as an expression vector (e.g., a mammalian expression vector, a yeast expression vector, an insect expression vector, or a bacterial expression vector), wherein the vector is optionally in a host cell that expresses said isolated monoclonal antibody or antigen-binding fragment thereof.

13. A pharmaceutical composition comprising the isolated monoclonal antibody or antigen-binding fragment thereof of any one of claims 1-10, or the mixture of claim 11,
optionally the pharmaceutical composition is formulated for intravenous administration, or for inhalational or oral administration; and/or,
optionally, the pharmaceutical composition is for treating a subject infected by SARS-CoV-2, and further comprises a pharmaceutically acceptable excipient or diluent.

14. A combination comprising the pharmaceutical composition of claim 13, and a second therapeutic agent effective to treat infection by SARS-CoV-2,
optionally, the second therapeutic agent comprises chloroquine or hydroxychloroquine, remdesivir, lopinavir and ritonavir, azithromycin, an immune system inhibitor to inhibits cytokine storm (such as an anti-IL-6 neutralizing antibody such as tocilizumab or sarilumab), CD24Fc, IFX-1, an anti-CCR5 antibody such as Leronlimab, DAS181, CM4620, an anti-IFNγ monoclonal antibody such as emapalumab, an IL-1R antagonist such as Anakinra, Danoprevir+Ritonavir, or combination thereof.

15. A method of treating or preventing a disease or condition arising from SARS-CoV-2 infection, the method comprising administering to a patient in need thereof an effective amount of the isolated monoclonal antibody or antigen-binding fragment thereof of any one of claims 1-10, the mixture of claim 11; the polynucleotide of claim 12, or the pharmaceutical composition of claim 13,
optionally, the method is for treating COVID-19 or a subject infected by SARS-CoV-2, wherein the method further comprises administering a second therapeutic agent; and/or,
optionally, said second therapeutic agent comprises chloroquine or hydroxychloroquine, remdesivir, lopinavir and ritonavir, azithromycin, an immune system inhibitor to inhibits cytokine storm (such as an anti-IL-6 neutralizing antibody such as tocilizumab or sarilumab), CD24Fc, IFX-1, an anti-CCR5 antibody such as Leronlimab, DAS181, CM4620, an anti-IFNγ monoclonal antibody such as emapalumab, an IL-1R antagonist such as Anakinra, Danoprevir+Ritonavir, Calquence (acalabrutinib), Xeljanz (tofacitinib), Jakafi (ruxolitinib), Olumiant (baricitinib), Ilaris (canakinumab), Otezla (apremilast), Mavrilimumab, or combination thereof.
